(19) United States
(12) Patent Application Publication
(10) Pub. No.: US 2015/0316548 A1
(43) Pub. Date: Nov. 5, 2015

(54) PROGRANULIN AS MARKER FOR AUTOIMMUNE DISORDERS

(71) Applicant: UNIVERSITAET DES SAARLANDES, Saarbruecken (DE)

(72) Inventors: Michael Pfreundschuh, Homburg/Saar (DE); Klaus-Dieter Preuss, Homburg/Saar (DE); Lorenz Thurner, Homburg/Saar (DE)

(21) Appl. No.: 14/647,796

(22) PCT Filed: Nov. 27, 2013
(86) PCT No.: PCT/EP2013/074829

§ 371 Date: May 27, 2015

Related U.S. Application Data
(60) Provisional application No. 61/730,772, filed on Nov. 28, 2012.

(51) Int. Cl. G01N 33/564

(52) U.S. Cl. CPC G01N 33/564; G01N 2440/14; G01N 2800/24

(57) ABSTRACT

The present invention provides for means and method for the detection and/or the quantification of anti-progranulin-autoantibodies in a biological sample of a subject. The present invention also provides for means and method for the detection and/or the quantification of hyper-phosphorylated progranulin in a biological sample of a subject. The present invention further provides for means and methods for the detection of an aberrant conversion pathway of progranulin into granulines in a biological sample of a subject. In fact, the presence of anti-progranulin-autoantibodies and/or hyper-phosphorylated progranulin and/or an aberrant conversion pathway may be indicative that the subject may be suffering from an autoimmune disorder.

### FIELD OF THE INVENTION

The present invention provides for a method for the detection and/or the quantification of anti-progranulin-autoantibodies in a biological sample of a subject. The presence of anti-progranulin-autoantibodies is indicative that the subject may be suffering from an autoimmune disorder. Hence, the presence of anti-progranulin-autoantibodies allows assessing whether or not a subject suffers from an autoimmune disorder. The present invention also provides for a method for the detection and/or the quantification of phosphorylated progranulin in a biological sample of a subject. The presence of phosphorylated progranulin may alternatively and/or in addition be indicative that the subject suffers from an autoimmune disorder. The present invention further provides for a method for the detection of an aberrant conversion pathway of progranulin into granulins in a biological sample of a subject. The presence of an aberrant conversion pathway of progranulin may alternatively and/or in addition be indicative that the subject suffers from an autoimmune disorder. Also provided are kits for performing the methods of the inventions. Also provided is progranulin for use in the treatment of autoimmune diseases.

### BACKGROUND OF THE INVENTION

Progranulin is a secreted glycoprotein and is expressed in neurons, neuroglia, chondrocytes, epithelial cells and leukocytes (Toh H et al. J Mol Neurosci 2011 November; 45(3):538-48). It is a precursor protein with an N-terminal signal peptide and seven granulin motifs. Each of these granulin motifs contains 12 cysteins, which are responsible for 6 disulfide bridges in every granulin (Bateman A et al. Bioessays 2009 November; 31(11):1245-54) Diverse functions of progranulin and several receptors and interaction partners of progranulin have been identified so far, as recently reviewed by van Damme et al. With regard to autoimmune diseases, the most interesting action of progranulin is its strong anti-inflammatory effect, which is mediated by its high affinity towards TNF-α-receptors 1 and 2 with a strong inhibitory effect on them. The strength of this anti-inflammatory effect was comparable to the effect of the synthetic TNF-blocker “Etanercept” in mouse models (Tang et al., 2011 Science.)

The demonstration of anti-neutrophil cytoplasmic antibodies (ANCAs) is a diagnostic hallmark supporting the diagnosis of vasculitis. Historically, ANCAs were identified coincidentally during studies of the role of anti-nuclear antibodies (ANA) in glomerulonephritis. ANCA-associated vasculitides (AAV) constitute a heterogeneous group of small-vessel vasculitides, comprising granulomatosis with polyangiitis (GPA, formerly known as Wegener granulomatosis), Churg-Strauss syndrome (CSS) and microscopic polyangiitis (MPA). Granulomatosis with polyangiitis is a chronic inflammatory and autoimmune disease, starting with granulomatous inflammation of the upper and/or lower respiratory tract and evolving into a generalized proteinase 3 (PR3)-ANCA associated vasculitis. Churg-Strauss syndrome can be easily distinguished from the other ANCA-associated vasculitides by the pentad of asthma or other atopic diseases, elevated IgE, eosinophilia, P-ANCA and small-vessel vasculitis. A pathogenetic role of PR3-ANCAs was demonstrated in an acute lung-injury model of TNF-alpha primed rat lungs, which were perfused with PR3-ANCA (HATTAR K, OPPERMANN S, ANKELE C et al. c-ANCA-induced neutrophil-mediated lung injury: a model of acute Wegener's granulomatosis. Eur Respir J 2010; 36:187-195). The pathogenicity of myeloperoxidase (MPO)-ANCAs was demonstrated in vivo in a passive transfer mouse model, which resulted in the development of pauci-immune focal necrotizing and crescentic glomerulonephritis (XIAO H, HEERINGA P, HU P et al. Antineutrophil cytoplasmic autoantibodies specific for myeloperoxidase cause glomerulonephritis and vasculitis in mice. J Clin Invest 2002; 110:955-963). However, the wide spectrum of P-ANCA-associated autoimmune diseases cannot be explained by the action of P-ANCA alone. Another ANCA directed against human lysosome associated membrane proteine-2 (hLAMP-2) (KAIN R, TADEMA H, MCKINNEY E F et al. High prevalence of autoantibodies to hLAMP-2 in anti-neutrophil cytoplasmic antibody-associated vasculitis. J Am Soc Nephrol 2012; 23:556-566) was shown to induce typical phenotypes in animal models, yet the clinical relevance of this finding has been discussed controversially (ROTH A J, BROWN M C, SMITH R N et al. Anti-LAMP-2 antibodies are not prevalent in patients with antineutrophil cytoplasmic autoantibody glomerulonephritis. J Am Soc Nephrol 2012; 23:545-555). In non-ANCA associated primary systemic vasculitides, such as classical panarteritis nodosa, giant cell arteritis and Takayasu's arteritis, which generally affect medium to large vessels, there was—with the exception of anti-endothelium antibodies—little evidence for a role of B lymphocytes and antibodies in the pathogenesis of the respective diseases until recently. However, recent studies have suggested a more profound involvement of B lymphocytes in the pathomechanisms of large-vessel vasculitides. For example, the occurrence of high titers of autoantibodies directed against the N-terminal 27 aa of the ferritin heavy chain has been proposed as a highly sensitive diagnostic marker for active giant cell arteritis (BAERLECKEN N T, LINNEMANN A, GROSS W L et al. Association of ferritin autoantibodies with giant cell arteritis/polymyalgia rheumatica. Ann Rheum Dis 2012) and in Takayasu's arteritis the frequent occurrence of high numbers of circulating CD27⁺⁺ plasmablasts similar to active SLE point into the same direction (HOYER B F, MUMTAZ I M, LODDENKEMPER K et al. Takayasu arteritis is characterised by disturbances of B cell homeostasis and responds to B cell depletion with rituximab. Ann Rheum Dis 2012; 71:75-79).

To date, most autoantibodies in systemic vasculitides have been identified by chance rather than by systemic search. Previous studies with systematic approaches based on 2D immunoblotting of human umbilical cord endothelial cells, human lung extract and human arterial wall extract with sera from vasculitis patients, hinted to the existence of formerly unknown autoantigens, which could only be partially characterized by mass spectrometry. (CHANSEAUD Y, TAMBY M C, GUILPAIN P et al. Analysis of autoantibody repertoires in small- and medium-sized vessels vasculitides. Evidence for specific perturbations in polyarteritis nodosa, microscopic polyangiitis, Churg-Strauss syndrome and Wegener's granulomatosis. J Autoimmun 2005; 24:169-179 and GUILPAIN P, SERVETTAZ A, TAMBY M C et al. A combined SDS-PAGE and proteomics approach to identify target autoantigens in healthy individuals and patients with autoimmune diseases. Ann N Y Acad Sci 2007; 1109:538-549). By screening a protein macroarray a previous study had identified, amongst others, the lysosomal transmembrane protein 9B, a key regulator for TNFα activation by binding to recombinantly expressed monoclonal Fab antibodies derived from B cells, which had been isolated by Laser microdissection from ectopic lymphoid structures in granulomatous tissues from patients with granulomatosis with polyangiitis (THURNER L, MULLER A, CERUTTI M et al. Wegener's granuloma harbors B lymphocytes with specificities against a proinflammatory transmembrane protein and a tetraspanin. J Autoimmun 2011; 36:87-90).

However, despite the existence of a couple of diagnostic markers such as autoantibodies or auto-antigens which aid in the screening of patients that are suspected of acquiring or suffering from an autoimmune disorder, these is still a need for further markers in order to expand and/or improve diagnosis and, thus, prevention and/or treatment of autoimmune disorders. The technical problem underlying the present application is therefore to comply with this need.

Indeed, in order to discover novel autoantibodies/autoantigens such as in systemic vasculitides, the present inventors screened the sera of patients with ANCA-associated and ANCA-negative primary systemic vasculitides for autoantibodies using protein macroarrays derived from human fetal brain, T-cell, lung and colon cDNA expression libraries. To their surprise, the present inventors found autoantibodies to progranulin in samples from subjects suffering from an autoimmune disorder. In addition, they also identified hyper-phosphorylated progranulin in subjects suffering from an autoimmune disorder. Furthermore, they identified aberrant forms/fragments (granulins) that are, without being bound by theory, processed from hyper-phosphorylated progranulin.

These findings allow the present inventors to provide a package of at least three ways to screen and/or identify subjects that may be at a risk of acquiring an autoimmune disorder and/or that may already suffer from an autoimmune disorder: (i) detection of autoantibodies against progranulin, (ii) detection of hyper-phosphorylated progranulin, (iii) detection of aberrant forms/fragments of progranulin. Of course, each of these methods can be used alone or in combination with each other. Hence, the finding of the present inventors expands the means and methods in the screening and/or diagnosis of autoimmune disorders.

Almost needless to mention that the specificity of identified autoantibodies/auto-antigens/aberrant forms of progranulin was demonstrated by studying patients suffering from a large spectrum of autoimmune and infectious diseases as well as healthy controls with a wide range of age.

### SUMMARY OF THE INVENTION

The present invention relates to a method for detecting and/or quantifying anti-progranulin-autoantibodies in a biological sample. The method is useful for the assessment, prediction and/or the diagnosis of autoimmune diseases.

The present invention relates to a method for detecting and/or quantifying hyper-phosphorylated progranulin in a biological sample. The method is useful for the assessment, prediction and/or the diagnosis of autoimmune diseases. Preferably, the hyper-phosphorylation is on a serine that corresponds to the serine residue at position 81 with respect to the progranulin reference sequence SEQ ID NO: 1 which is as follows (note that the S81 residue is marked):

```
SEQ ID NO: 1:
    1 MWTLVSWVAL TAGLVAGTRC PDGQFCPVAC CLDPGGASYS CCRPLLDKWP TTLSRHLGGP
  61 CQVDAHCSAG HSCIFTVSGT [symbol]  SCCPFPEAV ACGDGHHCCP RGFHCSADGR SCFQRSGNNS
121 VGAIQCPDSQ FECPDFSTCC VMVDGSWGCC PMPQASCCED RVHCCPHGAF CDLVHTRCIT
181 PTGTHPLAKK LPAQRTNRAV ALSSSVMCPD ARSRCPDGST CCELPSGKYG CCPMPNATCC
241 SDHLHCCPQD TVCDLIQSKC LSKENATTDL LTKLPAHTVG DVKCDMEVSC PDGYTCCRLQ
301 SGAWGCCPFT QAVCCEDHIH CCPAGFTCDT QKGTCEQGPH QVPWMEKAPA HLSLPDPQAL
361 KRDVPCDNVS SCPSSDTCCQ LTSGEWGCCP IPEAVCCSDH QHCCPQGYTC VAEGQCQRGS
421 EIVAGLEKMP ARRASLSHPR DIGCDQHTSC PVGQTCCPSL GGSWACCQLP HAVCCEDRQH
481 CCPAGYTCNV KARSCEKEVV SAQPATFLAR SPHVGVKDVE CGEGHFCHDN QTCCRDNRQG
541 WACCPYRQGV CCADRRHCCP AGFRCAARGT KCLRREAPRW DAPLRDPALR QLL
```

The present invention relates to a method for detecting an aberrant conversion pattern of progranulin into granulin(s) in a biological sample.

Non-aberrant progranulin conversion pattern entail the cleavage of progranulin into fragments, the N-terminal fragment having an apparent molecular weight of 45 kDa as measured in SDS PAGE. The N-terminal fragment contains granulin G, F, B and A. The C-terminal fragment contains granulin C, D and E. It has an apparent molecular weight of 35 kDa. (FIG. 16a). This progranulin conversion pattern has been also disclosed in Zhu et al. [11]).

The aberrant conversion pattern found by the present inventors is characterized by the cleavage of progranulin, preferably an hyperphosphorylated progranulin at a different cleavage site to generate a N-terminal fragment (aberrant cleavage product) having an apparent molecular weight of 55 kDa as determine with SDS PAGE. The N-terminal fragment contains granulin G, F, B and A and C. The C-terminal fragment contains granulin D and E. The C-terminal fragment has an apparent molecular weight of 25 kDa (FIG. 16b).

The method of the invention which detects said progranulin aberrant conversion pattern or aberrant conversion products is useful for the prediction and/or the diagnosis of autoimmune diseases. Preferably, the method detects an aberrant pattern comprising a cleaving product of progranulin having an apparent molecular weight of 55 kDa±5 as is determinable by SDS PAGE or any other suitable method such as 2D gel electrophoresis. The aberrant pattern comprises also a cleaving product of progranulin having an apparent molecular weight of 25 kDa±5 as is determinable by SDS PAGE or any other suitable method such as 2D gel electrophoresis. (FIG. 16). The SDS PAGE, is a gradient SDS PAGE gel electrophoresis and can be done under reducing or non-reducing conditions, preferably under reducing conditions.

Preferably, the method detects the cleaving product of progranulin having an apparent molecular weight of 55 kDa as is determinable by SDS PAGE or any other suitable method such as 2D gel electrophoresis or IEF.

The cleaving product of progranulin, having an apparent molecular weight of 55 kDa, as is determinable by SDS PAGE or any other suitable method such as 2D gel electrophoresis or IEF may be hyper-phosphorylated or non-hyper-phosphorylated.

The cleaving product of progranulin comprises the granulins G, F, B, A, C. Preferably, this cleaving product has an apparent molecular weight of 55 kDa as is determinable by SDS PAGE or any other suitable method such as 2D gel electrophoresis. Preferably, this cleaving product is hyper-phosphorylated. Preferably the hyper-phosphorylation is on a serine. Preferably, hyper-the phosphorylated serine is the serine corresponding to S81 in SEQ ID NO: 1.

Hence, the present invention is directed to the use of progranulin or hyper-phosphorylated progranulin or variant or fragment thereof for the detection and/or the quantification of anti-progranulin-antibodies.

Hence, the present invention is directed to the use of anti-hyper-phosphorylated-progranulin-antibodies for the detection or the quantification of hyper-phosphorylated progranulin.

The present invention also relates to kits for carrying out the methods of the invention. A kit may comprise an anti-hyper-phosphorylated progranulin antibody or fabs thereof or an anti-progranulin antibody and a phospho-serine specific antibody (commercially available from Invitrogen, Acris, Millipore, etc.). While the first antibody will detect progranulin, irrespective of whether or not it is phosphorylated, the second antibody will detect hyper-phosphorylated progranulin, the latter being indicative of an autoimmune disorder as described herein. Preferably, the anti-hyper-phosphorylated progranulin antibody or fabs thereof are directed specifically against hyperphosphorylated (Ser 81) progranulin.

The kit used in the detection and/or quantification of anti-progranulin-antibodies may comprise a solid support coated with at least one progranulin antigen or a hyper-phosphorylated progranulin antigen. Preferably the hyper-phosphorylated progranulin antigen comprises a hyper-phosphorylated serine corresponding to S81 of SEQ ID NO 1.

The kit used in the detection and/or quantification of hyper-phosphorylated progranulin may comprise a solid support with at least an anti-hyper-phosphorylated progranulin antibody or fab thereof.

Alternatively, the kit may comprise a solid support with at least an anti-progranulin antibody or fab thereof and may contain a phosphor-serine specific antibody.

In a further aspect, the present invention is directed to hyper-phosphorylated progranulin or hyper-phosphorylated progranulin peptides/fragments or variant thereof. Preferably, hyper-phosphorylated progranulin or hyper-phosphorylated progranulin peptide/fragment or variant thereof is phosphorylated at the serine corresponding to S81 of SEQ ID NO: 1.

In an embodiment, the present invention is directed to anti-progranulin-antibodies or antigen-binding fragments thereof. Preferably, the antibodies or antigen-binding fragments thereof bind to the N-terminal 112 amino acid region of SEQ ID NO: 1. Preferably, the antibodies or antigen-binding fragments thereof bind to the N-terminal 112 amino acid region (or fragment thereof) of the amino acid sequence of SEQ ID NO: 1. Said region may contain a hyper-phosphorylated serine. Preferably, the serine is the serine corresponding to S81 within SEQ ID NO: 1.

In a further aspect the present invention is directed to progranulin or variant or fragment thereof for the treatment of autoimmune diseases.

It must be noted that as used herein, the singular forms “a”, “an”, and “the”, include plural references unless the context clearly indicates otherwise. Thus, for example, reference to “an antibody” includes one or more of such different antibodies and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or sometimes when used herein with the term “having” or could even be replaced by consisting of.

When used herein “consisting of” excludes any element, step, or ingredient not specified in the claim element. When used herein, “consisting essentially of” does not exclude materials or steps that do not materially affect the basic and novel characteristics of the claim. In each instance herein any of the terms “consisting essentially of” and “consisting of” may be replaced with each other.

As used herein, the conjunctive term “and/or” between multiple recited elements is understood as encompassing both individual and combined options. For instance, where two elements are conjoined by “and/or”, a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or” as used herein.

Several documents are cited throughout the text of this specification. Each of the documents cited herein (including all patents, patent applications, scientific publications, manufacturer's specifications, instructions, etc.), whether supra or infra, are hereby incorporated by reference in their entirety. To the extent the material incorporated by reference contradicts or is inconsistent with this specification, the specification will supersede any such material. Nothing herein is to be construed as an admission that the invention is not entitled to antedate such disclosure by virtue of prior invention.

### FIGURES

FIG. 1: Progranulin-antibodies in different primary systemic vasculitides. The columns represent the percentage of patients in each of the vasculitis entities and subgroups, who were progranulin-antibody-positive at least at one timepoint in the course of their disease. The numbers above each column show the number of progranulin antibody-positive patients and the number of all patients in each entity/subgroup.

FIG. 2 shows the titres of progranulin antibodies in different autoimmune diseases. a) Continuous black line with black rhombi: plasma of a patient with microscopic polyangiitis, titer 1:3200. b) Interrupted black line with black triangles: plasma of a patient with active generalized ANCA-positive granulomatosis with polyangiitis, titer 1:3200. c) Continuous black line with black circles: plasma of classical panarteritis nodosa, titer 1:400. d) Interrupted black line with black quadrates: plasma of active systemic lupus erythematosus, titer 1:3200. e) Continuous grey line with grey triangles: plasma of a patient with rheumatoid arthritis, 1:800. f) Interrupted grey line with grey circles: plasma of a healthy control (negative at a starting dilution of 1:100).

FIG. 3 shows anti-Progranulin autoantibodies in rheumatoid arthritis, systemic lupus erythematodes and control groups (healthy, obesity, residential home patient, melanoma and sepsis). The columns represent the percentage of patients and healthy controls who were progranulin-antibody positive at least once during the course of their disease. The numbers above each column show the relative frequency of progranulin antibody-positive patients in the respective autoimmune disorder.

FIG. 4 shows the results of the determination of the anti-progranulin antibody binding epitope region by ELISA where fragments of different lengths and full-length PGRN were expressed recombinantly with C-terminal FLAG-tags and used as coat. Lane a: SLP2 full-length clone expressed in HEK293 with C-terminal FLAG-tag as negative control; lane b: full-length progranulin (1 aa to 593 aa); lane c: signal peptide (3-24 aa) with overlapping C-terminally amino acids; lane d: signal peptide, linker region 1 and overlapping amino (14-68 aa); lane e: GRN G (aa 62 to 112); lane f: GRN F (aa 128 to 178); lane g: a part of GRN F to the end of GRN E (aa 168 aa to 593). ELISAs with coated fragments containing parts of the N-terminal 128 aa resulted in clearly detectable reactivities.

FIG. 5 shows the association of anti-PGRN-autoantibody status and the plasma levels of progranulin in systemic vasculitides, systemic lupus erythematosus and rheumatoid arthritis. Lane a: healthy controls (n=18). The median progranulin plasma level obtained of the healthy controls was set at 100%; lane b: patients with systemic vasculitides who were progranulin-antibody negative (n=7); lane c: matched patients with systemic vasculitides who were progranulin-antibody positive (n=7); lane d: patients with systemic lupus erythematosus, who were progranulin-antibody negative (n=7); lane e: patients with systemic lupus erythematosus, who were progranulin-antibody positive (n=7); lane f: patients with rheumatoid arthritis, who were progranulin-antibody negative (n=7); lane g: patients with rheumatoid arthritis, who were progranulin-antibody positive (n=9). Data are presented as 25th (box), 50th (median), and 75th percentiles (box) and range (line). Statistical analysis revealed highly significant differences in the progranulin plasma levels of progranulin-antibody positive vasculitis patients compared to progranulin-antibody negative vasculitis patients (Mann-Whitney test: p=0.002) and to healthy controls (Mann-Whitney test: p<0.001), as well between progranulin-antibody positive patients with systemic lupus erythematosus compared to progranulin antibody negative patients with systemic lupus erythematosus (Mann-Whitney test: p=0.001) and healthy controls (Mann-Whitney test: p<0.001) and between progranulin-antibody positive patients with rheumatoid arthritis and progranulin-antibody negative patients with rheumatoid arthritis (Mann-Whitney test: p=0.001) and healthy controls (Mann-Whitney test: p<0.001). No significant differences were detected between progranulin antibody negative patients with systemic vasculitides, systemic lupus erythematosus or rheumatoid arthritis and the healthy control group.

FIG. 6 is the Western immunoblot of plasma progranulin in progranulin-antibody-positive and negative vasculitis patients. Lane 1, 2 and 3: plasma of healthy controls. Lane 4, 5 and 6: plasma of progranulin antibody positive patients with vasculitis. Lane 7, 8 and 9: plasma of progranulin antibody negative patients with vasculitis. Equal amounts of plasma were run on a standard 10% SDS polyacrylamide gel.

FIG. 7 is a plot showing the sensitivity of WEHI-S cells to TNF-α in dependency of progranulin-antibody status of added plasma. The absorbance of colored formazan at 450 nm, which is metabolite from tetrazolium salt produced only by living cells, indicates the viabilitiy of cells (EZ4U, Biomedica). To WEHI-S cell cultures, plasma of progranulin-antibody positive patients, plasma of matched antibody negative patients and healthy controls were added in different dilutions before the administration of TNF-α. The administration of progranulin-antibody positive plasma, results in lower adsorption at 450 nm which means less reduction of tetrazolium salt to formazan in viable cells, or more cytotoxicity in WEHI-S cells. Thus, the administration of progranulin-antibody containing plasma renders cells more sensitive to TNF-α. This difference is still detectable in plasma diluted 1:64.

FIG. 8 shows the additional negatively charged isoform of progranulin in progranulin-antibody positive patients. In isoelectric focusing every progranulin-antibody positive patient had an additional second band of progranulin with a different electric charge. Healthy controls (first lane) and autoimmune patients with negative progranulin-antibody status (last lane) did not have this additional and differentially electrically charged progranulin isoform.

FIG. 9 shows that the alkaline phosphatase treatment of aberrant progranulin reveals hyperphosphorylation. Fractions of the sera of two progranulin antibody positive patients (V8: SLE and ED22: ulcerative colitis) were treated with alkaline phosphatase before isoelectric focusing. These pretreated fractions had lost the aberrantly charged progranulin isoform in contrast to untreated fractions. This indicates a hyperphosphorylated state of the aberrant progranulin isoform.

FIG. 10 is the IEF of recombinantly expressed progranulin fragments in LCLs of progranulin-antibody positive patients and controls. Recombinantly in LCLs of a progranulin-antibody positive patient (V8) and in a healthy control (G7) expressed, FLAG-tagged progranulin-fragments of different lengths were subjected to IEF. The hyperphosphorylated wasn't detectable in the signal peptide, but in the fragments 1-27 112aa, 1-178aa and 1-251aa. In the longer fragments (1-251aa and 1-593) several different IEF gel bands were visible, reflecting cleaving products of progranulin. Like this the localization of the hyperphosphorylation site could be narrowed down to area of 24-112aa.

FIG. 11 a) and b) show the prevalence of the hyperphosphorylated progranulin isoform in different cell types in progranulin-antibody positive patients. Cell fractions of progranulin-antibody positive patients a) V328 (a) and V331 (b) were sorted and lysates of these cell fractions were subjected to IEF. As positive controls served lysates of whole blood cells of progranulin-antibody positive patients and respectively of healthy controls. The strongest gel bands of the hyperphosphorylated progranulin isoform appeared in the granulocytes and monocytes. Weak gel bands appeared in T- and B-lymphocytes. In erythrocytes neither the normal nor the hyperphosphorylated progranulin isoform were detectable.

FIGS. 12 a) and b): IEF of patients samples undergone progranulin-antibody seroconversions a) The IEFs performed of whole blood cell lysates of 11 patients (1-11) with positive and negative controls. From every patient several sera had been collected over the last years in the context of follow up diagnostics of their autoimmune disease. Every patient had once been progranulin-antibody positive. Apart from two of them (3 and 9), all underwent seroconversion of the progranulin-antibody status and were progranulin-antibody negative at the point of time, when the blood sample for the IEF was taken. The nine patients, who underwent seroconversion to progranulin-antibody negativity, showed only the normal progranulin isoform. Only in IEF of the two patients (3 and 9), who were still progranulin-antibody positive, appeared the additional hyperphosphorylated progranulin isoform. b) ELISA for progranulin-antibody status and IEF for hyperphosphorylation status were performed with plasma and whole blood cell samples, longitudinally obtained for a period of 10 months from an individual patient with rheumatoid arthritis and secondary Sjögren' syndrome. In the first two months, the patient was still seronegative and the additionally hyperphosphorylated progranulin wasn't present. At the end of the 10 months, the patient had a flare of the disease and was positive for the hyperphosphorylated progranulin and for progranulin-autoantibodies.

FIGS. 13 a) to e): Identification of PKCβ1 as the responsible class of kinases for hyperphosphorylated progranulin a) The application of Staurosporin at 5 μM, at 2.5 μM and of Ellagic Acid at a concentration of 50 μM to the cell-culture of LCL of a progranulin-antibody positive patient, led to the disappearance of the hyperphosphorylated progranulin isoform, and as well to a change in the electrical charge of the “normal” progranulin isoform; b) This change in the electrical charge of “normal” progranulin appeared as well after the administration of Staurosporin at 5 μM, at 2.5 μM and of Ellagic Acid at a concentration of 50 μM to the cell-culture of LCL of a healthy control. c) The application of Staurosporin at lower concentrations (30 nM or 10 nM) to the LCL cell cultures, led only to the disappearance of the hyperphosphorylated progranulin isoform, but not to a change in electric charge of the “normal” progranulin isoform as it is induced by higher concentrations of Staurosporin e.g. 2.5 μM. d) When adding Bis-indolyl-maleimide I (BIM I) to the cell cultures, only relative high concentrations (20 nM) led to the disappearance of the hyperphosphorylated isoform. This indicated PKCβ as the responsible kinase. In contrast Rottlerin at different concentrations was not possible to inhibit the responsible kinase. e) Finally this could be verified by the application of PKCβ-inhibitor. High concentrations (21 nM), but not low concentrations (5 nM) of PKCβ-inhibitor led to the disappearance of the hyperphosphorylated isoform. This identified PKCβ-1 as the kinase sought after, which is responsible for hyperphosphorylation of progranulin.

FIG. 14 a) to b): Identification of PP1 as the responsible phosphatase. a) Phosphatase inhibitors at specific concentrations or DMSO only as control were administrated for three days to cultured LCLs derived from a healthy donor and from a patient with hyperphosphorylated progranulin. Subsequently LCL lysates were subjected to IEF. Lanes (1) and (4) show the IEF of the LCL from a healthy control and a progranulin-antibody positive patient cultured without phosphatase inhibitors. Lanes (2) and (5) show the disappearance of the normal and the appearance of the hyperphosphorylated progranulin isoform in the IEF as a result of the addition of serine/threonine phosphatase inhibitor cocktail 1 (Sigma-Aldrich; dilution 1:1000) to the LCLs of healthy control and progranulin-antibody positive autoimmune patient. In contrast to this the addition/incubation of tyrosine phosphatase inhibitors to the LCLs did not result in any change, i.e. appearance/disappearance of the progranulin isoforms (lanes (3) and (6)). b) The first two lanes show negative and positive controls indicating the normal and the hyperphosphorylated progranulin isoforms. The further lanes show the administration of following phosphatase inhibitors to LCL-lysates from healthy controls (top) and from progranulin-antibody positive patients (bottom): 1) DMSO, 2) of serine/threonine inhibitor cocktail 1, 3) of NIPP1 at 10 pM, 4) of okadaic acid at 10 nM, 5) of okadaic acid at 0.1 nM and of okadaic acid at 500 nM. Serine/threonine phosphatase inhibitor cocktail 1, NIPP1 at a concentration of 10 pM and okadaic acid at concentrations of 500 nM and 10 nM led to the appearance of the hyperphosphorylated progranulin isoform and the disappearance of the normal progranulin isoform. Lower concentrated ocadaic acid 0.1 nM didn't change the phosphorylation state. This proved that PP1 is the responsible phosphatase for Ser81.

FIG. 15 a) to c): a) Aberrant conversion of hyperphosphorylated progranulin into mature granulins (n-terminal pattern). Whole blood cell lysates of a healthy control, a progranulin-antibody positive patient with psoriatic arthritis (V236) and a progranulin-antibody negative patient with rheumatoid arthritis (V238) were subjected to Western blot analysis. The progranulin antibody positive patient shows an aberrant pattern of conversion with a bigger fragment of 55 kDa. The normal conversion pattern with a fragment of 45 kDa coexisted, but was obviously weaker than in the controls. As detection antibody patient's plasma was utilized, which is directed against the N-terminal 112aa. 15 b) Aberrant conversion of progranulin from antibody-positive patients into mature granulins (c-terminal pattern). Whole blood cell lysates of two healthy controls, two progranulin-antibody positive patients with autoimmune disease and two progranulin-antibody negative patients with autoimmune disease were subjected to Western blot analysis with a rabbit anti human progranulin antibody directed against the C-Terminus (BIOSS). The progranulin antibody positive patients showed an additional aberrant pattern of conversion, with a smallerconversion fragment of progranulin of 25 kDa instead of 35 kDa in the controls. Additionally to this a 15 kDa fragment appeared. 15 c) Aberrant conversion displayed with phospho/non-phospho-specific fabs. A western blot for the N-terminal conversion pattern was repeated with whole blood cell lysates from the same patients as in 2a). Here specific fabs against the normal progranulin (Ser81) at the left and against hyperphosphorylated progranulin (pSer81) at the right were utilized. The aberrant conversion is only visible in the right blot, in which the N-terminal fragments of hyperphosphorylated progranulin isoform were detected. The non hyperphosphorylated normal progranulin-isoform has the same conversion pattern in progranulin-antibody positive patients as in healthy controls.

FIG. 16 a) and b): Scheme of normal and aberrant conversion of progranulin in healthy controls and patients with hyperphosphorylated progranulin. a) Western-blot analyses of whole blood cell and LCL lysates of healthy controls and of progranulin-antibody negative autoimmune patients, i.e. no hyperphosphorylated progranulin, the normal progranulin conversion pattern according to Zhu et al. appeared [11]. b) Western-blot analyses of whole blood cell and LCL lysates of progranulin-antibody positive patients, i.e. with hyperphosphorylated progranulin, suggested a shift in the first conversion in from the linker region in-between Granulin A and Granulin C to the linker region in-between Granulin C and Granulin D.

FIG. 17: Aberrant conversion of hyperphosphorylated progranulin into mature granulins. Lysates of LCL from a healthy control and progranulin-antibody positive patient, which had previously been transfected with n-terminally HIS-tagged and c-terminally FLAG-tagged progranulin, were subjected to Western blot analysis. The progranulin-antibody positive patient shows an aberrant pattern of conversion

FIG. 18: ELISA of hyperphosphorylated progranulin (pPGRN) plasma levels. pPGRN was detectable by ELISA only in plasma of patients, who were as well positive for hyperphosphorylated progranulin in the IEF of whole blood cell lysates (lanes 1 to 4).

FIG. 19: Flow cytometric analysis of patients with and without Ser81 PGRN. Overlay of fly cytometric analysis of PBMCs of a patient without hyperphosphorylated PGRN (FIG. 19a) and a patient with hyperphosphorylated PGRN (FIG. 19b). Grey lines (marked with “1”) indicate anti-non-phosphorylated PGRN Fab, black lines (marked with “2”) indicate anti-phosphorylated PGRN Fab.

### DETAILED DESCRIPTION OF THE INVENTION

The present inventors have identified anti-progranulin-autoantibodies as a marker for assessing, predicting and/or diagnosing autoimmune diseases. Indeed, they have detected anti-progranulin autoantibodies in sera of patients affected by autoimmune diseases such as systemic vasculitides, systemic Lupus erythematosus (SLE), rheumatoid and psoriatic arthritis etc., but not in healthy controls.

The present inventors have also identified progranulin and/or hyper-phosphorylated progranulin as auto-antigen in autoimmune diseases such as systemic vasculitides, systemic Lupus erythematosus, rheumatoid and psoriatic arthritis etc. but not in healthy controls.

The present inventors have furthermore demonstrated that anti-progranulin-autoantibodies have a neutralizing effect on progranulin plasma levels, which is of particular interest in the light of progranulin's function as a potent direct inhibitor of TNFR-1&2. When testing sera samples of the same individuals during the course of autoimmune diseases in different activity states, the anti-progranulin-autoantibody status changes and the prevalence of anti-progranulin-autoantibodies is significantly associated with active disease. Beneath this neutralizing effect of the autoantibodies on the progranulin-plasma level, a functional effect of anti-progranulin-autoantibodies is demonstrated in vitro, leading to a strongly increased sensitivity of WEHI-S cells to TNF-α.

Furthermore, the inventors have found and confirmed the presence of (a) hyper-phosphorylated isoform(s) of progranulin in all anti-progranulin-autoantibody positive patients. To the best knowledge of the inventors, this hyper-phosphorylation is exclusive in all progranulin-autoantibody positive patients and has not been observed before.

Preferably, the hyper-phosphorylation site is located in the affinity region of the autoantibodies (1-112 AA) in Grn G (S81). Without being bound to any theory, this suggests that this secondary modification is the reason for the breakdown of self-tolerance and that hyper-phosphorylated progranulin constitutes a so called neo-(auto)antigen.

Moreover, the inventors have identified PKCβ-I as the responsible kinase for this hyperphosphorylation. Hence, inhibitors of PKCβ-I are envisaged for use in a method of the prevention and/or treatment of autoimmune disorders. Such inhibitors are considered beneficial in preventing PKCβ-I from aberrant phosphorylation of progranulin. Accordingly, such inhibitors preferably prevent or interfere with the interaction between PKCβ-I and progranulin, thereby inhibiting phosphorylation, in particular at a serine residue corresponding to S81 of SEQ ID NO: 1.

Moreover, the inventor has identified PP1 as the phosphatase responsible for the de-phosphorylation of the hyper-phosphorylated progranulin or of the aberrant cleaving product thereof. The aberrant forms of progranulin are disclosed in the present application and are part of the invention. PP1 preferably dephosphorylate the serine residue corresponding to S81 of SEQ ID NO: 1.

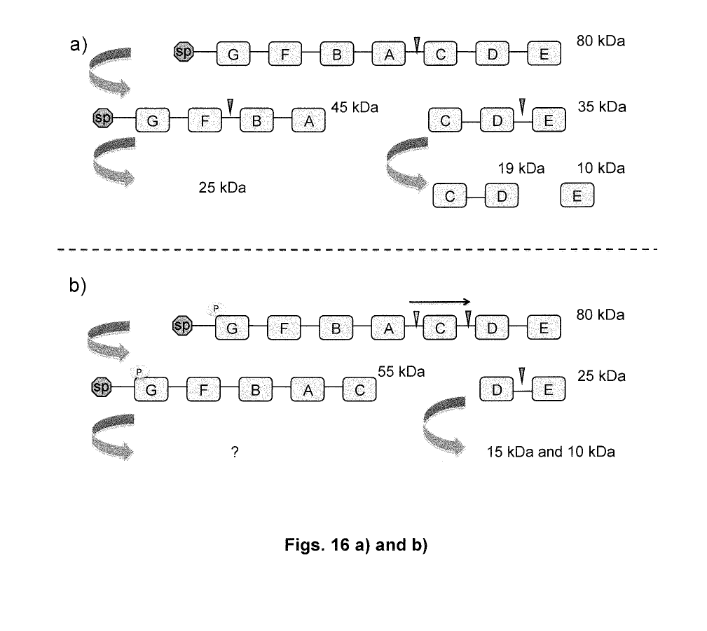

In addition to this, the present inventors have found that the conversion patter of progranulin into mature granulins is aberrant in progranulin-antibody positive patients compared to progranulin-autoantibody negative subject. The first steps of the progranulin conversion pattern into mature granulin in a healthy subject or progranulin-autoantibody negative subject are depicted in FIG. 16 a. The first steps of the aberrant progranulin conversion pattern are depicted in FIG. 16 b. In both figures, the skilled person understands that the letters G, F, B, A, C, D and E indicate the parts of the sequence that would be cleaved into the mature granulins. In anti-progranulin-autoantibody positive patients the aberrant conversion pattern prevails over the normal conversion pathway. Substantially, there appeared longer N-terminal and a shorter C-terminal conversion fragments in anti-progranulin-autoantibody patients. In the aberrant pattern, the cleavage site is shifted from the conversion region between A-C to the conversion region between C-D (see FIG. 16 b.) Indeed, the present inventors have observed that the electrophoretic pattern (Western blotting) of anti-progranulin autoantibody positive patients is different from anti-progranulin autoantibody-negative subjects. The presence of aberrant cleaving products (fragment) of progranulin has been identified. E.g. an aberrant cleaving product has been identified to have an apparent molecular weight of 55kDA as measure with SDS PAGE. This cleaving product may be hyper-phosphorylated or non-hyper-phosphorylated. An additional aberrant cleaving product has been identified to have a apparent molecular weight of 25 kDa as measure with SDS PAGE.

### DEFINITIONS

Throughout the specification, several terms are employed and are defined in the following paragraphs.

As used herein, the term “progranulin” has a general meaning in the art. Progranulin is also known with the terms “proephitelin”, “granulin epithelin precursor (GEP), PC-cell derived growth factor (PCDGF), acrogranin, proepithelin (PEPI) or CLN11. Progranulin is a secreted glycoprotein and is expressed in neurons, neuroglia, chondrocytes, epithelial cells and leukocytes. It is a precursor protein with an N-terminal signal peptide and seven granulin motifs (granulins A, B, C, D, E, F and G). Each of these granulin motifs contains 12 cysteins, which are responsible for 6 disulfide bridges in every granulin. Diverse functions of progranulin and several receptors and interaction partners of progranulin have been identified so far, as recently reviewed by van Damme et al. Many examples of nucleotide and amino acid progranulin sequences are available, for example, in the database provided by the National Center for Biotechnology Information (NCBI) (see http://www.ncbi.nlm.nih.gov/). Preferably, the progranulin sequence of accession number NP_{—}002078.1 is preferred. One example for progranulin/proepithelin is the amino acid sequence SEQ ID NO: 1. In the context of the present invention SEQ ID NO: 1 is preferred as reference sequence.

The preferred nucleotide sequence of progranulin, in particular for serving as a reference sequence is NP_{—}002078.2 or SEQ ID NO: 3, the latter being preferred.

The term “progranulin” when used herein also includes fragments, homologs or variants of progranulin. Such homologs are defined by way of their homology to the amino acid sequence of SEQ ID NO: 1. Accordingly, a progranulin includes all proteins that have a sequence homology or sequence identity of more than 40%, 50%, 60%, 70% 80%, 85%, 90%, or 95% in relation to the progranulin shown in SEQ ID NO: 1.

The term “homology” as used herein in its usual meaning and includes identical amino acids as well as amino acids which are regarded to be conservative substitutions (for example, exchange of a glutamate residue by a aspartate residue) at equivalent positions in the linear amino acid sequence of two proteins that are compared with each other. The term “sequence identity” or “identity” as used in the present invention means the percentage of pair-wise identical residues—following homology alignment of a sequence of a polypeptide of the present invention with a sequence in question—with respect to the number of residues in the longer of these two sequences. The percentage of sequence homology or sequence identity is preferably determined herein using the program BLASTP, version blastp 2.2.5 (Nov. 16, 2002; cf. Altschul, S. F. et al. (1997) Nucl. Acids Res. 25, 3389-3402). The percentage of homology is based on the alignment of the entire polypeptide sequences (matrix: BLOSUM 62; gap costs: 11.1; cutoff value set to 10⁻³), using the progranulin of SEQ ID NO: 1 as reference in a pairwise comparison. It is calculated as the percentage of numbers of “positives” (homologous amino acids) indicated as result in the BLASTP program output divided by the total number of amino acids selected by the program for the alignment. It is noted in this connection that this total number of selected amino acids can differ from the length of the progranulin of SEQ ID NO: 1.

A “fragment” of progranulin includes at least 6, 7, 8, 9, 10, 15, 20, 30, 35, 40, 50, 60, 70, 80, 90, 100, 150, 200, 250, 300, 350, 400, 450 or 500 amino acids of a progranulin protein. Preferably, such a fragment is antigenic. A “variant” of a progranulin protein includes, for example, a splice variant, any allelic variant (e.g., caused by a SNP or the like), isoforms, etc., as long as such a variant shares homology with the progranulin of SEQ ID NO: 1 as described herein. A preferred isoform of progranulin is a hyper-phosphorylated form thereof as described herein or an aberrantly processed form as is also described herein. Of course, the term “progranulin” also includes glycosylated and/or phosphorylated forms thereof. When used herein, the term “hyper-phosphorylated” in the context of progranulin proteins means that a progranulin contains a phosphate residue at an amino acid where at the corresponding amino acid in a “normal” progranulin no such phosphate residue is present. “Normal” progranulin means a progranulin present in healthy subject or in progranulin autoantibody-negative subject. Such a normal progranulin is known to be phosphorylated at the C-terminal. The hyper-phosphorylated residue is preferably at a position corresponding to position 81 of the progranulin shown in SEQ ID NO: 1. A hyper-phosphorylated fragment or hyper-phosphorylated variant of progranulin is a fragment that contains a residue (amino acid) which is not phosphorylated in normal progranulin. A hyper-phosphorylated amino acid is an aminoacid which is phosphorylated in the hyper-phosphorylated progranulin, but which is not phosphorylated in “normal” progranulin. For example a hyper-phosphorylated serine is a serine which is phosphorylated in a hyper-phosphorylated progranulin but not in a “normal” progranulin. For example S81 of SEQ ID NO is phosphorylated in hyper-phosphorylated progranulin but not in “normal” progranulin.

The term “position” when used in accordance with the disclosure means the position of either an amino acid within an amino acid sequence depicted herein or the position of a nucleotide within a nucleic acid sequence depicted herein. The term “corresponding” as used herein also includes that a position is not only determined by the number of the preceding nucleotides/amino acids. Accordingly, the position of a given amino acid in accordance with the disclosure which may be substituted may very due to deletion or addition of amino acids elsewhere in a progranulin. Similarly, the position of a given nucleotide in accordance with the present disclosure which may be substituted may vary due to deletions or additional nucleotides elsewhere in a progranulin 5′-untranslated region (UTR) including the promoter and/or any other regulatory sequences or gene (including exons and introns).

Thus, under a “corresponding position” in accordance with the disclosure it is preferably to be understood that nucleotides/amino acids may differ in the indicated number but may still have similar neighbouring nucleotides/amino acids. Said nucleotides/amino acids which may be exchanged, deleted or added are also comprised by the term “corresponding position”

Specifically, in order to determine whether an amino acid residue of the amino acid sequence of a progranulin is different from a wild-type progranulin (SEQ ID NO: 1 serves as reference or wild-type sequence) corresponds to a certain position in the amino acid sequence of a wild-type progranulin, a skilled artisan can use means and methods well-known in the art, e.g., alignments, either manually or by using computer programs such as BLAST2.0, which stands for Basic Local Alignment Search Tool or ClustalW or any other suitable program which is suitable to generate sequence alignments. Accordingly, a wild-type progranulin can serve as “subject sequence” or “reference sequence”, while the amino acid sequence of a progranulin different from the wild-type progranulin described herein serves as “query sequence”. The terms “reference sequence” and “wild type sequence” are used interchangeably herein.

A preferred nucleotide sequence of progranulin is herein indicated as SEQ ID NO: 3 and is as follows:

atgtggaccct ggtgagctgg gtggccttaa cagcagggct
ggtggctgga acgcggtgcc cagatggtca gttctgccct
gtggcctgct gcctggaccc cggaggagcc agctacagct
gctgccgtcc ccttctggac aaatggccca caacactgag
caggcatctg ggtggcccct gccaggttga tgcccactgc
tctgccggcc actcctgcat ctttaccgtc tcagggactt
ccagttgctg ccccttccca gaggccgtgg catgcgggga
tggccatcac tgctgcccac ggggcttcca ctgcagtgca
gacgggcgat cctgcttcca aagatcaggt aacaactccg
tgggtgccat ccagtgccct gatagtcagt tcgaatgccc
ggacttctcc acgtgctgtg ttatggtcga tggctcctgg
gggtgctgcc ccatgcccca ggcttcctgc tgtgaagaca
gggtgcactg ctgtccgcac ggtgccttct gcgacctggt
tcacacccgc tgcatcacac ccacgggcac ccaccccctg
gcaaagaagc tccctgccca gaggactaac agggcagtgg
ccttgtccag ctcggtcatg tgtccggacg cacggtcccg
gtgccctgat ggttctacct gctgtgagct gcccagtggg
aagtatggct gctgcccaat gcccaacgcc acctgctgct
ccgatcacct gcactgctgc ccccaagaca ctgtgtgtga
cctgatccag agtaagtgcc tctccaagga gaacgctacc
acggacctcc tcactaagct gcctgcgcac acagtggggg
atgtgaaatg tgacatggag gtgagctgcc cagatggcta
tacctgctgc cgtctacagt cgggggcctg gggctgctgc
ccttttaccc aggctgtgtg ctgtgaggac cacatacact
gctgtcccgc ggggtttacg tgtgacacgc agaagggtac
ctgtgaacag gggccccacc aggtgccctg gatggagaag
gccccagctc acctcagcct gccagaccca caagccttga
agagagatgt cccctgtgat aatgtcagca gctgtccctc
ctccgatacc tgctgccaac tcacgtctgg ggagtggggc
tgctgtccaa tcccagaggc tgtctgctgc tcggaccacc
agcactgctg cccccagggc tacacgtgtg tagctgaggg
gcagtgtcag cgaggaagcg agatcgtggc tggactggag
aagatgcctg cccgccgggc ttccttatcc caccccagag
acatcggctg tgaccagcac accagctgcc cggtggggca
gacctgctgc ccgagcctgg gtgggagctg ggcctgctgc
cagttgcccc atgctgtgtg ctgcgaggat cgccagcact
gctgcccggc tggctacacc tgcaacgtga aggctcgatc
ctgcgagaag gaagtggtct ctgcccagcc tgccaccttc
ctggcccgta gccctcacgt gggtgtgaag gacgtggagt
gtggggaagg acacttctgc catgataacc agacctgctg
ccgagacaac cgacagggct gggcctgctg tccctaccgc
cagggcgtct gttgtgctga tcggcgccac tgctgtcctg
ctggcttccg ctgcgcagcc aggggtacca agtgtttgcg
cagggaggcc ccgcgctggg acgccccttt gagggaccca
gccttgagac agctgctgtg a

As used herein the term “antibody” refers to a polypeptide or protein capable of (specifically) binding a protein/polypeptide (also sometimes called antigen herein). An antibody typically binds an epitope or an antigenic determinant present on said antigen. “Antibody” refers to a polypeptide ligand substantially encoded by an immunoglobulin gene or immunoglobulin genes, or fragments thereof, which specifically binds and recognizes an epitope (e.g., an antigen). The recognized immunoglobulin genes include the kappa and lambda light chain constant region genes, the alpha, gamma, delta, epsilon and mu heavy chain constant region genes, and the myriad immunoglobulin variable region genes. Antibodies exist, e.g., as intact immunoglobulins or as a number of well characterized fragments produced by digestion with various peptidases. This includes, e.g., Fab′ and F(ab)′2 fragments. The term “antibody,” as used herein, also includes antibody fragments either produced by the modification of whole antibodies or those synthesized de novo using recombinant DNA methodologies. It also includes polyclonal antibodies, monoclonal antibodies, chimeric antibodies, humanized antibodies, or single chain antibodies. “Fc” portion of an antibody refers to that portion of an immunoglobulin heavy chain that comprises one or more heavy chain constant region domains, CH1, CH2 and CH3, but does not include the heavy chain variable region. The term “antibody” includes monoclonal, polyclonal, chimeric, humanized, human antibodies, animal antibodies such as mammalian antibodies such as dog, cat, camel, horse, caw, pig, monkey etc. antibodies, rodent antibodies such as mouse antibodies, antibody fragments. It also includes bispecific antibodies, diabodies, tetrabodies, domain antibodies, nanobodies and the like. As an alternative to an antibody, a scaffold selected from the group consisting of CTLA-4 (Evibody); lipocalin; Protein A derived molecules such as Z-domain of Protein A (Affibody, SpA), A-domain (Avimer/Maxibody); Heat shock proteins such as GroEL and GroES; transferrin (trans-body); ankyrin repeat protein (DARPin); peptide aptamer; C-type lectin domain (Tetranectin); human γ-crystallin and human ubiquitin (affilins); PDZ domains; scorpion toxin kunitz type domains of human protease inhibitors; and fibronectin (adnectin) may be used as antigen binding molecule. Thus, in a broader sense, an antigen binding domain can be applied in the methods of the present invention for binding progranulin.

An “autoantibody” applied in the present invention is an antibody produced by the immune system of a subject that is directed against one or more of the subject's own proteins (self-antigens or autoantigens including neo-(auto)antigens). The autoantibody applied in the embodiments of the present invention may be indicative for an autoimmune disease.

The term “indicative for” when used in the context of an autoantibody applied in the embodiments of the present invention includes that an autoantibody, the presence or absence of which is determined in the embodiments of the present invention, is typically present in a subject having a autoimmune disorder. “Typically present” means that the autoantibody is usually associated with a autoimmune disorder. “Usually associated” includes a probability of more than 50%, preferably more than 60%, more preferably more than 70%, even more preferably more than 80% and particularly preferred more than 90 or 95%.

The term “indicative for” also includes that the autoantibody, the presence or absence of which is determined in the embodiments of the present invention is directed against, binds to, reacts with or recognizes auto-antigens, i.e., self-antigens of the subject that produced them. For example, by binding to, reacting with or recognizing an auto-antigen the presence of an autoantibody in a sample obtained from a subject can be determined. Likewise, by not binding to, not reacting with or not recognizing an auto-antigen the absence of an autoantibody in a sample obtained from a subject can be determined.

The term “indicative for” however also includes that (auto)antigens, the presence or absence of which is determined in some of the embodiments of the present invention is bound by an antibody directed against, reacts with or recognizes said antigens that may occur in a subject having an autoimmune disorder.

As used herein the term “progranulin antigen” or “progranulin antigenic peptide/fragment, refers to progranulin or fragment which is (specifically) recognized by an anti-progranulin-antibody. Progranulin antigen or “progranulin antigenic peptide/fragment may be also generated in vitro by e.g. genetic manipulation and produced in heterologous system (such as bacteria). As seen in the present invention “progranulin antigenic peptide/fragment” may be hyper-phosphorylated. Preferably, the “progranulin antigenic peptide/fragments” are hyper-phosphorylated on one or more serine. Preferably, the antigen which specifically recognized by the anti-progranulin-autoantibody comprises or consists of SEQ ID NO: 2 i.e. 1-112 N-terminal fragment of SEQ ID NO: 1 or fragments or variant thereof. SEQ ID NO: 2 is as follows:

1 mwtlvswval taglvagtrc pdgqfcpvac cldpggasys ccrplldkwp ttlsrhlggp
61 cqvdahcsag hsciftvsgt ssccpfpeav acgdghhccp rgfhcsadgr sc

Preferably, the fragments of SEQ ID NO: 2 have a minimum length of 10 amino acids. Preferably the fragments of SEQ ID NO: 2 comprise the serine corresponding to the S81 or S82 in SEQ ID NO: 1, preferably, the serine corresponding to the S81. Optionally but preferably, the serine corresponding to the S81 or S82 in SEQ ID NO: 1 is phosphorylated. More preferably, the serine corresponding to S81 is phosphorylated.

Preferably, a “progranulin antigenic peptide/fragment” is phosphorylated on the serine corresponding to the S81 or S82 in SEQ ID NO: 1, preferably the serine corresponding to the S81. For example, a fragment can be the fragment consisting of aa70 to aa112 wherein S81 may be optionally but preferably phosphorylated.

“Autoantigen” is used to denote antigens (e.g. progranulin autoantigen) for which the presence or absence of antibodies in a sample of a subject is determined. An “autoantigen” in the sense of the present invention is an endogenous tissue or cell constituent that has the ability to interact with autoantibodies and may cause an immune response, thereby causing or contribute to cause, inter alia, an autoimmune disorder. Said term includes also neo-autoantigens.

An autoantigen or antigen can be an entire mature form of a protein, such as a protein referred to as a target antigen or can be a precursor, processed form, unprocessed form, isoforms, variant, a fragment thereof that includes an epitope, or allelic variant thereof, providing that the modified, processed, or variant for of the protein has the ability to bind autoantigens present in samples from subjects. It can be isolated from a subject, recombinantly or synthetically produced.

Autoantigens are contemplated to include any fragments thereof, in particular, immunologically detectable fragments. They are also meant to include immunologically detectable products of proteolysis of the proteins, as well as processed forms, post-translationally modified forms, such as, for example, “prep” “pro,” or “prepro” forms of markers, or the “prep” “pro,” or “prepro” fragment removed to form the mature protein, as well as allelic variants and splice variants of the autoantigens. An “autoantigen” refers to a molecule, such as a protein, endogenous to the host that is recognized by an autoantibody. The autoantigen may, for example, also be a complex of proteins, DNA or RNA, either single- or double-stranded or a glycoprotein.

As used herein the term “peptide” or “polypeptide” are used interchangeably with protein herein and refer to a sequence of contiguous amino acids linked by peptide bonds. As used herein, the term “protein” refers to a polypeptide that can also include post-translational modifications that include the modification of amino acids of the protein and may include the addition of chemical groups or biomolecules that are not amino acid-based. The terms apply to amino acid polymers in which one or more amino acid residue is an analog or mimetic of a corresponding naturally occurring amino acid, as well as to naturally occurring amino acid polymers. Polypeptides can be modified, e.g., by the addition of carbohydrate residues to form glycoproteins. The terms “polypeptide,” “peptide” and “protein” include glycoproteins, as well as non-glycoproteins. The terms “polypeptide” or “peptide” include peptide of several amino acids (e.g. 10 to 200aa, 10 to 120aa). More specifically a peptide according to the invention has a length of at most 200, 150, 100, 50, 40, 35, 30, 25, 20, 15 or 10 aa.

A “variant” (for example, an allelic variant or splice variant) of an autoantigen, as used herein, in particular progranulin, also refers to an amino acid sequence that is altered with respect to the referenced polypeptide or protein by one or more amino acids. In the present invention, a variant of the autoantigen of the invention retains the antigenicity, or antibody-binding property, of the referenced autoantigen. In preferred aspects of the invention, a variant of an autoantigen can be bound by the same population of autoantibodies that are able to bind the referenced autoantigen, which is preferably progranulin, more preferably progranulin as shown in SEQ ID NO: 1. Preferably a variant of an autoantigen has at least 50% or 60% identity to the referenced autoantigen over a sequence of at least 15 amino acids. More preferably a variant of an autoantigen is at least 70% identical to the referenced autoantigen over a sequence of at least 15 amino acids. Autoantigen variants can be, for example, at least 80%, at least 90%, at least 95%, or at least 99% identical to referenced autoantigen over a sequence of at least 15 amino acids. Autoantigen variants of the invention can be, for example, at least 80%, at least 90%, at least 95%, or at least 99% identical to referenced autoantigen over a sequence of at least 20 amino acids. The variant may have “conservative” changes, wherein a substituted amino acid has similar structural or chemical properties (e.g., replacement of leucine with isoleucine). A variant may also have “non-conservative” changes (e.g., replacement of glycine with tryptophan). Analogous minor variations may also include amino acid deletions or insertions, or both. Guidance in determining which amino acid residues may be substituted, inserted, or deleted without abolishing immunological reactivity may be found using computer programs well known in the art.

A fragment of an autoantigen, in particular progranulin, that includes an epitope recognized by an autoantibody can also be at least 5, 6, 7, 8, 9, 10, 11, 12, 13, 14, 15, 20, 25, 30, 35, 40, 45, 50, 75, 100, 150, 200, 250, 300, 350, 400, 450, or 500 amino acids in length. The fragment can also be between 5, 10, 15, 20, 25, 50, 75, 100, 150, 200, or 250 and one amino acid less than the entire length of an autoantigen. Typically, such epitopes are characterized in advance such that it is known that autoantibodies for a given autoantigen recognize the epitope. Methods for epitope mapping are well known in the art. An “epitope” is a site on an antigen, such as an autoantigen disclosed herein, recognized by an autoantibody.

Normally autoantibodies are routinely eliminated by the immune system's self-regulatory process—probably through the neutralization of autoantibody-producing lymphocytes before they mature. However, at times this process fails, and antibodies that react to self-constituents proliferate. Generally, autoantibodies damage body tissues by bringing about the phagocytosis (ingestion) or lysis (bursting) of healthy cells. Autoantibodies also interfere with the normal functioning of cells. Moreover, by attacking the subject's own cells, tissues, and/or organs, autoantibodies cause, for example, inflammation and/or damage. The causes of autoantibody production are varied and not well understood.

Being directed to, binding to, reacting with or recognizes means that the autoantibody applied in the embodiments of the present invention specifically binds to autoantigens as described herein. The term “specifically” in this context means that the autoantibody reacts with its counterpart autoantigen but essentially not with another protein. A “counterpart autoantigen” is an autoantigen which is bound by an autoantibody.

As used herein the language “detecting” means determining the presence or the absence of the selected (bio)marker in a sample by assaying the sample and optionally “reading” and/or “interpreting” the result obtained by assaying the sample. In the context of the present invention, a marker is, for example, the anti-progranulin-autoantibodies, the progranulin, progranulin antigenic peptides/fragments, progranulin antigen, hyper-phosphorylated progranulin or progranulin antigenic peptides/fragments. Progranulin is cleaved in different granulin form A, B, C, D, E, F, G. Accordingly, a marker can be one of the granulin or association thereof. For example, association of granulins can be the forms G-F-B-A, C-D-E, G-F, B-A, C-D, G-F-B-A-C, D-E (FIG. 16).

As used herein the term “diagnosing” refers to methods by which the person skilled in the art can estimate or even determine whether a subject is suffering from a given disorder or condition. The person skilled in the art often makes a diagnosis on the basis of one or more diagnostic indicators, such as anti-progranulin autoantibodies, or hyper-phosphorylated progranulin or hyper-phosphorylated progranulin antigenic peptides/fragments the level (including presence or absence) of which is indicative of the presence or absence of an autoimmune disease.

As used herein the term autoimmune disease or disorder refers to conditions triggered by aberrant reaction of the immune system, in particular of the human immune system. Preferably, said autoimmune disorder is characterized by the occurrence of autoantibodies against an autoantigen, in particular an autoantibody against progranulin. It may also be characterized by the occurrence of (auto)antigens, in particular a hyper-phosphorylated form of progranulin. Furthermore, in the context of the present invention, an autoimmune disorder may be characterized by an aberrantly processed form of progranulin as described herein.

Examples of autoimmune diseases are vasculitis, arthritides, autoimmune diseases of the connective tissue, inflammatory bowel diseases, autoimmune diseases of the liver and the bile duct, autoimmune disease of the thyroid gland, dermatologic autoimmune diseases, neurologic immune diseases, Diabetes type I. Vasculites are selected from medium to small vessel vasculitis or large vessel vasculitis, arthritides are selected from seronegative and seropositive rheumatoid arthritis, psoriatic arthritis, Bechterew's disease, juvenile idiopathic arthritis; inflammatory bowl diseases are selected from Crohn's disease or ulcerative colitis; diseases of the liver and the bile duct are selected from autoimmuno-hepatitis, primary biliary cirrhosis, primary sclerosing Cholangitis; autoimmune diseases of the thyroid gland are selected from Hashimoto's thyreoiditis, Grave's disease; autoimmune diseases of the connective tissue are selected from systemic lupus erythematosus disease, Sjörgen's syndrome, scleroderma, dermato- and poly-myositis, Sharp syndrome, systemic sclerosis and CREST syndrome; neurologic autoimmune diseases are selected from multiple sclerosis, chronic inflammatory demyelating polyneuropathy (CIDP), myasthenia gravis. Medium to small vasculitis is selected from classical panarteritis nodosa, granulomatosis with polyangiitis, microscopic panarteritis, Churg-Strauss syndrome Behcet's disease and the large vessel vasculitis is selected from giant cell arteritis, polymyalgia rheumatica, Takayasu's arteritis.

A “patient” in the context of the present invention is an individual subject, which is tested for the presence or absence of an autoimmune disorder or a non-autoimmune disorder, respectively according to the present invention. A patient tested for an autoimmune disorder or a non-autoimmune disorder can have one or more indicators of such a disorder as described herein, or can be screened for the presence or absence of said disorder in the absence of any indicators of said disorder. As used herein an “individual suspected of having said disorder” or “subject suspected of having said disorder” can have one or more indicators (such a clinical indicators) of said disorder or may be at an increased risk of developing said disorder due to age, environmental and/or nutritional factors, or genetic factors or can be part of a population routinely screened for said disorder in the absence of any indicators of said disorder.

A “subject” in the context of the present invention is a mammal, in particular a human. “Mammal” for purposes of the present invention refers to any animal classified as a mammal, including human, domestic and farm animals, nonhuman primates, and any other animal that has mammary tissue. A mammal includes human, rodents such as mouse, rat or rabbit, dog, cat, chimpanzee, horse, pig, etc., with human being preferred. A subject also includes human and veterinary patients, with human patients being preferred.

The term “marker” or “biomarker” as used herein refers generally to a molecule i.e. gene, protein, polypeptide, peptide etc., the presence or the absence of which in a biological sample can be detected by standard method in the art as well as those disclosed herein and is predictive or denotes a condition of the patient/subject from which the sample was obtained. In the context of the present invention, a biomarker is preferably an autoantibody against progranulin, a hyper-phosphorylated form of progranulin or an aberrantly processed form of progranulin.

In accordance with the present invention by the term “sample” is intended any sample, preferably biological sample, obtained from a subject, cell line, tissue culture, or other source. The sample may contain autoantibodies, polynucleotides or polypeptides or portions thereof. Biological samples include body fluids (such as blood, serum, plasma, urine, saliva, synovial fluid and spinal fluid) and tissue sources found to contain autoantibodies. Methods for obtaining tissue biopsies and body fluids from subjects are well known in the art. Generally, a biological sample which includes proteins, in particular antigens, autoantibodies and polynucleotides is preferred as a source. Other preferred samples are whole blood, serum, plasma or synovial fluid, with plasma or serum being most preferred. Whole blood cells and lysate cell, in particular whole blood lysate cells are also a preferred embodiment of the present invention.

“Presence” (in all its grammatical forms) of progranulin autoantibody or progranulin antigen or aberrantly processed form of progranulin means that a progranulin autoantibody or progranulin antigen or aberrantly processed form of progranulin which is indicative for at least one autoimmune disorder is immunologically detectable. This means that the methods of the present invention are particularly suited for the screening of the presence of an autoantibody against progranulin, a hyper-phosphorylated progranulin and/or an aberrantly processed form of progranulin.

Any immunoassay known in the art can be used for immunologically detecting the binding of an autoantibody to an autoantigen or the binding of an antigen to an antibody. Immunoassays are well known to the skilled artisan. Methods for carrying out such assays as well as practical applications and procedures are summarized in related textbooks. Examples of related textbooks are Tijssen, P., In: Practice and theory of enzyme immunoassays, Burdon, R. H. and v. Knippenberg, P. H. (eds.), Elsevier, Amsterdam (1990), pp. 221-278, and various volumes of Colowick, S. P. and Caplan, N. O. (eds.), Methods in Enzymology, Academic Press, dealing with immunological detection methods, especially volumes 70, 73, 74, 84, 92 and 121.

Immunoassays, for example, include precipitation (particularly immunoprecipitation), electrochemiluminescence (electro-generated chemiluminescence), RIA (radioimmunoassay), ELISA (enzyme-linked immunosorbent assay), sandwich enzyme immune tests, electrochemiluminescence sandwich immunoassays (ECLIA), dissociation-enhanced lanthanide fluoro immuno assay (DELFIA), scintillation proximity assay (SPA), turbidimetry, nephelometry, latex-enhanced turbidimetry or nephelometry, solid phase immune tests, and mass spectrometry such as SELDI-TOF, MALDI-TOF, or capillary electrophoresis-mass spectrometry (CE-MS). Any electrophoretic technic is suitable for the purpose of the present method for detecting hyperphosphorylated progranulin and the aberrant conversion pathway.

Furthermore, suitable immunoassays include microplate ELISA-based methods, fully-automated or robotic immunoassays (available for example on Elecsys™ or Cobas™ analyzers), and latex agglutination assays (available for example on Roche-Hitachi™ analyzers).

Typically, in the present invention an immunoassay is an assay in which a anti-progranulin-autoantibody specifically binds a progranulin autoantigen to provide for the detection and/or quantification of an autoantibody or an assay in which a progranulin antigen binds to a progranulin antibody to provide for the detection and/or quantification of said antigen.

“Absence” (in all its grammatical forms) of an autoantibody or autoantigen or aberrantly processed form of progranulin means that an autoantibody or antigen or aberrantly processed form of progranulin which is indicative for at least one autoimmune disorder is not immunologically detectable. Accordingly, the autoantibody is not bound to an autoantigen Likewise, “absence” means that the progranulin antigen is not bound by an antibody contained in the test area as described herein. Also, “absence” means that no aberrantly processed form of progranulin can be detected. As mentioned above, any immunoassay known in the art can be used. This means that the methods of the present invention are particularly suited for the screening of the absence of an autoantibody against progranulin, a hyper-phosphorylated progranulin and/or an aberrantly processed form of progranulin.

“Is not immunologically detectable” when used herein preferably includes that a signal generated by the binding of a progranulin autoantibody to a progranulin autoantigen or the binding of a progranulin antigen to an antibody as described herein is below a predetermined test-area specific threshold value, i.e., the signal generated by the binding of an autoantibody to an autoantigen or by the binding of an antigen to an antibody is negative.

A test area may be an area on a solid surface such as on a plate or stick or the like. It may also be a slot of a microtiter plate, etc. In the broadest possible sense, a test area is a zone where an autoantibody binds to its antigen or an autoantigen binds to an antibody.

In a preferred aspect of the methods of the present invention a signal is classified as positive when it is above a predetermined test-area-specific cut-off value and is classified as negative when it is below a predetermined test-area-specific cut-off value, wherein a positive signal indicates that either an autoantibody or autoantigen is present in a sample.

A “cut-off value” is the boundary value (or limit value) of the amount and/or concentration of an autoantibody or antigen that determines as to when a signal is positive or negative.

In the present case, the cut-off value indirectly reflects the amount and/or concentration of an autoantibody or antigen, since on the test area a certain amount and/or concentration of an autoantigen or antibody is present to which autoantibodies or antigens contained in a sample from a subject bind. The certain amount and/or concentration of an autoantigen or antibody is predetermined.

Thus, a “signal” reflects the result of an (auto)antibody-(auto)antigen or antibody-antigen reaction which includes the binding of an (auto)antibody to an auto-antigen present or the binding of an antibody to antigen or antibody-antigen. Accordingly, the term “signal” includes the result of an autoantibody-autoantigen reaction or vice versa, which includes the binding of an autoantibody to an autoantigen or vice versa. Likewise, this term includes the result of an antigen-antibody reaction which includes the binding of an antigen to an antibody.

If the cut-off value is exceeded (or above a predetermined threshold value), this is indicative of the presence of an autoantibody or antigen indicative for an autoimmune disorder and, thus, a signal or the result of an autoantibody-autoantigen reaction or antigen-antibody reaction is classified as positive.

However, if the cut-off value is deceeded (or below a predetermined threshold value), this is indicative of the absence of an autoantibody or antigen indicative for an autoimmune disorder and, thus, a signal or the result of an autoantibody-autoantigen reaction or antigen-antibody reaction is classified as negative.

A signal can be determined by way of the signal provided by the signal generating group of an autoantibody-specific receptor which is described herein below. The signal can be any signal which is detectable by, for example, spectroscopic, photochemical, biochemical, immunochemical, or chemical means as described herein below. Preferably, the antigen to which the autoantibody binds provides a signal or a molecule that binds the autoantibody provides a signal.

A signal can also be determined by way of the signal provided by the signal generating group of an antigen-specific receptor which is described herein below.

By using predetermined test-area specific threshold values it is possible to optimize these cut-off values according to the assay requirements. Therefore, the skilled artisan can shift the cut-off values to achieve a higher specificity or a higher sensitivity of the methods of the present invention and kits applying these methods. The threshold value or cut off value can be determined by different procedures as known from virological testing. The preferred embodiment is to measure a sufficient set of patient samples with an autoimmune disorder (“diseased collective” or “disease group”) and patient samples not having an autoimmune disorder (“control collective” or “control group” or “control sample”) and to fix the cut-off as required for the clinical situation. In the context of the present invention, the disease group has been determined to have either autoantibodies against progranulin and/or hyper-phosphorylated progranulin and/or aberrantly processed progranulin forms, while the control group has none of these.

For example, a reference value may be determined as the median or as specific percentile (i.e., 0.90 or 0.95) of the measured levels in a population of subjects not having an autoimmune disease, preferably one which is associated with autoantibodies against progranulin, hyper-phosphorylated progranulin, and/or aberrantly processed forms of progranulin. Evaluating the levels in further individuals or patients, e.g. in cohort studies, can help to refine the known levels or ratios.

Also, a level known in the art for a particular autoantibody may be used as a “reference value”. The person skilled in the art is familiar with the concept of reference values (or “normal values”) for biomarkers such as autoantibodies indicative for an autoimmune disorder. In particular, the term reference value may relate to the actual value of the level in one or more control samples or it may relate to a value derived from the actual level in one or more control samples. Preferably, samples of at least 50, more preferably at least 100, more preferably at least 500, most preferably at least 1000 subjects.

In the simplest case, the reference value is the same as the level measured in the control sample or the average of the levels measured in a multitude of control samples. However, the reference value may also be calculated from more than one control sample. E.g., the reference value may be the arithmetic average of the level in control samples representing the control status (e.g. healthy, particular condition, or particular disease state). Preferably, the reference value relates to a range of values that can be found in a plurality of comparable control samples (control samples representing the same or similar disease status), e.g. the average one or more times the standard deviation.

Similarly, the reference value may also be calculated by other statistical parameters or methods, for example as a defined percentile of the level found in a plurality of control samples, e.g. a 90%, 95%, 97.5%, or 99% percentile. The choice of a particular reference value may be determined according to the desired sensitivity, specificity or statistical significance (in general, the higher the sensitivity, the lower the specificity and vice versa). Calculation may be carried out according to statistical methods known and deemed appropriate by the person skilled in the art.

The terms “control” or “control sample” or the like are known to the person skilled in the art. Preferably, the “control” relates to an experiment or test carried out to provide a standard, against which experimental results (e.g. the measured level(s) in a subject) can be evaluated. In the present context, the standard preferably relates to the level of the autoantibody of interest indicative for the presence of an autoimmune disorder, in particular an autoantibody against progranulin. Thus, a “control” is preferably a sample taken to provide such a standard. E.g., the control sample may be derived from one or more healthy subjects, or from one or more subjects representative of a non-autoimmune disorder. In the present context, the standard preferably relates to the level of the hyper-phosphorylated progranulin or hyper-phosphorylated progranulin antigenic peptide/fragment of interest indicative for the presence of an autoimmune disorder. Thus, a “control” is preferably a sample taken to provide such a standard. E.g., the control sample may be derived from one or more healthy subjects, or from one or more subjects representative of a non-autoimmune disorder. In the present context, the standard preferably relates to the normal/conventional conversion pattern of progranulin into granulins. Thus, a “control” is preferably a sample taken to provide such a standard. E.g., the control sample may be derived from one or more healthy subjects, or from one or more subjects representative of a non-autoimmune disorder.

It is a preferred embodiment of the invention to use an optimized multivariate cut-off for the underlying autoantibodies the presence or absence of which is to be determined in accordance with the methods of the present invention so as to discriminate state A from state B, e.g. a subject having an autoimmune disorder from an otherwise healthy subject. Accuracy of a diagnostic method is best described by its receiver-operating characteristics (ROC) (see especially Zweig, M. H., and Campbell, G., Clin. Chem. 39 (1993) 561-577). The ROC graph is a plot of all of the sensitivity/specificity pairs resulting from continuously varying the decision thresh-hold over the entire range of data observed.

The clinical performance of a laboratory test depends on its diagnostic accuracy, or the ability to correctly classify subjects into clinically relevant subgroups. Diagnostic accuracy measures the test's ability to correctly distinguish two different conditions of the subjects investigated. Such conditions are for example health and disease or benign versus malignant disease.

In each case, the ROC plot depicts the overlap between the two distributions by plotting the sensitivity versus 1-specificity for the complete range of decision thresholds. On the y-axis is sensitivity, or the true-positive fraction [defined as (number of true-positive test results)/(number of true-positive+number of false-negative test results)]. This has also been referred to as positivity in the presence of a disease or condition. It is calculated solely from the affected subgroup. On the x-axis is the false-positive fraction, or 1-specificity [defined as (number of false-positive results)/(number of true-negative+number of false-positive results)]. It is an index of specificity and is calculated entirely from the unaffected subgroup. Because the true- and false-positive fractions are calculated entirely separately, by using the test results from two different subgroups, the ROC plot is independent of the prevalence of disease in the sample. Each point on the ROC plot represents a sensitivity/1-specificity pair corresponding to a particular decision threshold. A test with perfect discrimination (no overlap in the two distributions of results) has an ROC plot that passes through the upper left corner, where the true-positive fraction is 1.0, or 100% (perfect sensitivity), and the false-positive fraction is 0 (perfect specificity). The theoretical plot for a test with no discrimination (identical distributions of results for the two groups) is a 45° diagonal line from the lower left corner to the upper right corner. Most plots fall in between these two extremes. (If the ROC plot falls completely below the 45° diagonal, this is easily remedied by reversing the criterion for “positivity” from “greater than” to “less than” or vice versa.) Qualitatively, the closer the plot is to the upper left corner, the higher the overall accuracy of the test.

One convenient goal to quantify the diagnostic accuracy of a laboratory test is to express its performance by a single number. The most common global measure is the area under the ROC plot. By convention, this area is always >0.5 (if it is not, one can reverse the decision rule to make it so). Values range between 1.0 (perfect separation of the test values of the two groups) and 0.5 (no apparent distributional difference between the two groups of test values). The area does not depend only on a particular portion of the plot such as the point closest to the diagonal or the sensitivity at 90% specificity, but on the entire plot. This is a quantitative, descriptive expression of how close the ROC plot is to the perfect one (area=1.0).

“Electrophoresis” as used in the present context is a separations technique that is based on the mobility of ions in an electric field. Positively charged ions migrate towards a negative electrode and negatively-charged ions migrate toward a positive electrode. “Electrophoresis based assay” as used in the present context refers to any assay based on this technology. Examples of electrophoresis based assays are for example: electro-focusing, isoelectric-focusing, gel electrophoresis, western (immune) blotting, 2D gel-electrophoresis etc.

SDS-PAGE electrophoresis is a known method to determining molecular weight of a protein or fragment thereof based on electrophoretic separation. For example, gradient SDS PAGE gel electrophoresis is used for determining the molecular weight of a protein or fragment. For example SDS PAGE, preferably, gradient SDS PAGE gel electrophoresis can be used in the context of the Western immune-blotting technology to determine the molecular weight of a protein or fragment thereof. SDS PAGE, gradient SDS PAGE gel electrophoresis and SDS PAGE, preferably, gradient SDS PAGE gel electrophoresis in the context of the Western immune-blotting technology are used to determine the conversion pattern of progranulin into granulins and the aberrant conversion pattern of progranulin into granulin as disclosed herein.

Detection Methods

A) Auto-Progranulin Antibodies Detection

A first aspect, the present invention relates to a in vitro method for detecting the presence of anti-progranulin-autoantibody in a biological sample of a subject. Optionally, the method includes the quantification of the anti-progranulin-autoantibody. Preferably, the method is for the diagnosis of an autoimmune disease.

An embodiment of the first aspect relates to an in vitro method for the detection of anti-progranulin-autoantibodies in a biological sample characterized by the steps of:

a) contacting the biological sample
1) progranulin and/or antigenic peptide/fragment and/or variant thereof; or
2) hyperphosphorylated progranulin and/or hyperphosphorylated antigenic peptide/fragment and/or variant thereof or
3) a mixture of 1) and 2)

b) detecting the binding of anti-progranulin autoantibodies

1′) to said progranulin and/or to said antigenic peptide/fragment thereof and/or to said variant thereof or

2′) to said hyperphosphorylated progranulin and/or hyperphosphorylated antigenic peptide/fragment and/or variant thereof or

3′) to said mixture of 1) and 2); and

c) optionally detecting in the biological sample one or more autoantibodies or autoantigens which are indicative of an autoimmune disorder.

Preferably, the method is for the diagnosis of an autoimmune disease.

The skilled person understands that detecting the binding of anti-progranulin autoantibodies to the mixture of 1) and 2) means detecting the binding of anti-progranulin autoantibodies to the components of the mixture as specified in points 1) and 2).

Hence an embodiment of the first aspect of the invention relates to an in vitro method for the diagnosis of an autoimmune disease in a subject by detection of anti-progranulin-autoantibodies in a biological sample. The method is characterized by the steps of
a) contacting the biological sample with hyper-phosphorylated progranulin or hyper-phosphorylated fragment or hyper-phosphorylated variant thereof;
b) detecting the binding of anti-progranulin-autoantibodies to hyper-phosphorylated progranulin or hyper-phosphorylated peptide/fragment or hyper-phosphorylated variant thereof
   wherein the binding of anti-progranulin-autoantibodies to said hyper-phosphorylated progranulin or hyper-phosphorylated peptide fragment or hyper-phosphorylated variant thereof is indicative of an autoimmune disease.

Preferably, the method is for the diagnosis of an autoimmune disease.

Hence an embodiment of the first aspect of the invention relates to an in vitro method for the diagnosis of an autoimmune disease in a subject by detection of anti-progranulin-autoantibodies in a biological sample. The method is characterized by the steps of
a) contacting the biological sample with a mixture of
   1) progranulin and/or antigenic peptide/fragment and/or variant thereof; or
   2) hyperphosphorylated progranulin and/or hyperphosphorylated antigenic peptide/fragment and/or hyperphosphorylated variant thereof or
b) detecting the binding of anti-progranulin-autoantibodies to said progranulin or a peptide/fragment or a variant thereof or hyper-phosphorylated progranulin or hyper-phosphorylated peptide/fragment or hyper-phosphorylated variant thereof
   wherein the binding of anti-progranulin-autoantibodies to said progranulin or a fragment or a variant thereof or hyper-phosphorylated progranulin or hyper-phosphorylated fragment or hyper-phosphorylated variant thereof is indicative of an autoimmune disease.

Preferably, the method is for the diagnosis of an autoimmune disease.

The method optionally further comprises detecting in the biological sample one or more autoantibodies or autoantigens which are indicative of an autoimmune disorder.

The biological sample is preferably from an individual suspected of having or at a risk of an autoimmune disease. The presence of anti-progranulin-autoantibodies in the biological sample is indicative of an autoimmune disease. Any biological sample is contemplated by the present invention such as tissues or liquid sample. Preferred is a liquid sample such as serum, blood, plasma and/or cerebrospinal fluid. Preferred are also whole blood cells, lysate cells in particular whole blood lysate cells.

An autoimmune disease can be diagnosed according to the method of the invention. The autoimmune diseases are for example vasculitis, arthritides, autoimmune diseases of the connective tissue, inflammatory bowel diseases, autoimmune diseases of the liver and the bile duct, autoimmune disease of the thyroid gland, dermatologic autoimmune diseases, neurologic immune diseases, Diabetes type I. Vasculites are selected from medium to small vessel vasculitis or large vessel vasculitis, arthritides are selected from seronegative and seropositive rheumatoid arthritis, psoriatic arthritis, Bechterew's disease, juvenile idiopathic arthritis; inflammatory bowl diseases are selected from Crohn's disease or ulcerative colitis; diseases of the liver and the bile duct are selected from autoimmuno-hepatitis, primary biliary cirrhosis, primary sclerosing Cholangitis; autoimmune diseases of the thyroid gland are selected from Hashimoto's thyreoiditis, Grave's disease; autoimmune diseases of the connective tissue are selected from systemic lupus erythematosus disease, Sjörgen's syndrome, scleroderma, dermato- and poly-myositis, Sharp syndrome, systemic sclerosis and CREST syndrome; neurologic autoimmune diseases are selected from multiple sclerosis, chronic inflammatory demyelating polyneuropathy (CIDP), myasthenia gravis. Medium to small vasculitis is selected from classical panarteritis nodosa, granulomatosis with polyangiitis, microscopic panarteritis, Churg-Strauss syndrome Behcet's disease and the large vessel vasculitis is selected from giant cell arteritis, polymyalgia rheumatica, Takayasu's arteritis. In the methods of the invention, the detection of the anti-progranulin-autoantibodies is carried out by using a progranulin, in particular at least a progranulin antigen or progranulin antigenic peptide/fragment or variant thereof. Examples of progranulin antigen or antigenic progranulin peptide/fragment or variant thereof include but are not limited to different fragments of progranulin, in particular and preferably fragments of progranulin comprising or consisting of the N-terminal 120 aminoacids of the progranulin or the N-terminal 120 aminoacids of SEQ ID NO: 1 or the N-terminal 112 aminoacids of the progranulin or the N-terminal 112 aminoacids of SEQ ID NO 1 (i.e. SEQ ID NO 2). The progranulin antigen or progranulin antigenic peptide/fragment or variant thereof may be hyper-phosphorylated. In particular, they may be phosphorylated at the serine corresponding to S81 or S82 in reference SEQ ID NO: 1, preferably at the serine corresponding to S81.

For example, the progranulin or progranulin antigen or progranulin antigenic peptide/fragment or variant thereof comprises or consists of
a) the amino acid sequence set forth in SEQ ID NO: 1 or a fragment thereof or a variant thereof;
b) the amino acid sequence set forth in SEQ ID NO: 1 which is hyper-phosphorylated or a hyper-phosphorylated thereof or a hyper-phosphorylated variant thereof;
c) the N-terminal 120 amino acid region of progranulin or fragment/variant thereof; preferably the N-terminal 112 amino acid region of progranulin or fragment/variant thereof, preferably SEQ ID NO 2 or fragment/variant thereof.
d) the N-terminal 120 amino acid region of progranulin or fragment/variant thereof; preferably the N-terminal 112 amino acid region of progranulin or fragment/variant thereof, preferably SEQ ID NO 2 which are hyper-phosphorylated or a hyper-phosphorylated fragment/variant thereof.

The fragments have a length of 10-100, 10-80; 10-60, 10-50 aminoacids and contain a serine corresponding to S81 in SEQ ID NO 1.

Preferably, the progranulin or the progranulin antigenic peptide/fragment of variant thereof comprises a hyper-phosphorylated serine, preferably at least a hyper-phosphorylated serine. Preferably, the hyper-phosphorylated serine is the serine corresponding to S81 in SEQ ID NO: 1.

In another aspect, the present invention is directed to the use of progranulin or an antigenic peptide/fragment or variant thereof in the detection of anti-progranulin-autoantibodies. The use may be an in vitro use. Hence, the present invention is directed to the use of progranulin or an antigenic peptide/fragment or variant thereof for the diagnosis of an autoimmune disease. The autoimmune disease is as defined in paragraph [132] above.

In the progranulin use aspect of the invention, progranulin may have the amino acid sequence SEQ ID NO: 1 or a fragment thereof or a variant thereof. SEQ ID NO: 1 or a fragment thereof or a variant thereof may be hyper-phosphorylated. Preferably, the progranulin, the fragment or the variant thereof comprises a hyper-phosphorylated serine, preferably at least a hyper-phosphorylated serine. Preferably, the hyper-phosphorylated serine is the serine corresponding to position 81 or 82 in SEQ ID NO: 1. Preferably the hyper-phosphorylated serine is the serine corresponding to S81 in SEQ ID NO: 1.

Preferred fragments are those comprising or consisting of SEQ ID NO: 2 (N-terminal 112 amino acids sequence of the amino acid sequence of SEQ ID NO: 1) or fragment thereof. Preferably, the preferred fragments or the variants thereof comprise a hyper-phosphorylated serine, preferably at least a hyper-phosphorylated serine. Preferably, the hyper-phosphorylated serine is the serine corresponding to position 81 or 82 in SEQ ID NO: 1. Preferably, the hyper-phosphorylated serine is the serine corresponding to S81 in SEQ ID NO: 1. Preferred fragments are fragments encompassing a serine corresponding to S81 in SEQ ID NO: 1.

Hence the present invention is directed to composition comprising
1) progranulin and/or antigenic peptide/fragment and/or variant thereof; and
2) hyper-phosphorylated progranulin or hyperphosphorylated antigenic peptide/fragment or hyperphosphorylated variant thereof.

In the composition, preferably progranulin and/or the hyperphosphorylated progranulin comprises or consists of the amino acid sequence set forth in SEQ ID NO: 1 or an antigenic fragment thereof. Preferably, the antigenic peptide/fragment antigenic peptide/fragment and/or the hyperphosphorylated peptide/fragment comprises or consists of SEQ ID NO: 2 or fragment or variant thereof. In the composition, preferably the hyperphosphorylated progranulin or the antigenic peptide/fragment or variant thereof comprises a hyper-phosphorylated serine, preferably the serine in position 81 in SEQ ID NO: 1 or the serine corresponding to S81 of SEQ ID NO: 1.

B) Hyper-Phosphorylated Progranulin or an Antigenic Peptide/Fragment Thereof Detection

In another aspect, the present invention is directed to an in vitro method for detection of hyper-phosphorylated progranulin or a hyper-phosphorylated antigenic progranulin peptide/fragment or variant thereof in a biological sample. Optionally the method includes the quantification of the hyper-phosphorylated progranulin or a hyper-phosphorylated antigenic progranulin peptide/fragment or variant thereof in a biological sample. Preferably, the method is for the diagnosis of an autoimmune disease.

Hence, the present invention is directed to an in vitro method for detection and/or quantification of hyper-phosphorylated progranulin or a hyper-phosphorylated antigenic progranulin peptide/fragment or variant thereof in a biological sample characterized by the steps of:
a) contacting the biological sample with an antibody or fab thereof which (specifically) binds to hyper-phosphorylated progranulin or to a hyper-phosphorylated progranulin antigenic peptide/fragment or hyper-phosphorylated variant thereof,
b) detecting the binding of said antibody or fab thereof to said hyper-phosphorylated progranulin or a hyper-phosphorylated progranulin antigenic peptide/fragment or hyper-phosphorylated variant thereof.

Hence, an embodiment of this aspect of the invention relates to an in vitro method for the diagnosis of an autoimmune disorder in a subject by detection of hyper-phosphorylated progranulin or a hyper-phosphorylated antigenic progranulin peptide/fragment or variant thereof in a biological sample. The method is characterized by the steps of:
a) contacting the biological sample with an antibody or fab thereof which (specifically) binds to hyper-phosphorylated progranulin or to a hyper-phosphorylated progranulin antigenic peptide/fragment or hyper-phosphorylated variant thereof,
b) detecting the binding of said antibody or fab thereof to said hyper-phosphorylated progranulin or a hyper-phosphorylated progranulin antigenic peptide/fragment or variant thereof.
   wherein the binding of said antibody or fab thereof to said hyper-phosphorylated progranulin or a hyper-phosphorylated progranulin antigenic peptide/fragment or hyper-phosphorylated variant thereof is indicative of an autoimmune disease.

According to the method of the invention, the presence of hyper-phosphorylated progranulin or a hyper-phosphorylated progranulin antigenic peptide/fragment or variant thereof in the biological sample is indicative of an autoimmune disease.

The method optionally further comprises detecting in the biological sample one or more autoantibodies or autoantigens which are indicative of an autoimmune disorder.

The biological sample is from an individual suspected of having or at a risk of an autoimmune disease and the presence of hyper-phosphorylated progranulin or hyper-phosphorylated peptide/fragment or variant thereof in the biological sample is indicative of an autoimmune disease. Any biological sample is contemplated by the present invention such as tissues or liquid sample. Preferred is a liquid sample such as serum, blood, plasma and/or cerebrospinal fluid. The biological sample is preferably from an individual suspected of having or at a risk of an autoimmune disease. The presence of anti-progranulin-autoantibodies in the biological sample is indicative of an autoimmune disease. Any biological sample is contemplated by the present invention such as tissues or liquid sample. Preferred is a liquid sample such as serum, blood, plasma and/or cerebrospinal fluid. Preferred are also whole blood cells, lysate cells in particular whole blood lysate cells.

An autoimmune disease can be diagnosed according to the method of the invention. The autoimmune diseases are for example vasculitis, arthritides, autoimmune diseases of the connective tissue, inflammatory bowel diseases, autoimmune diseases of the liver and the bile duct, autoimmune disease of the thyroid gland, dermatologic autoimmune diseases, neurologic immune diseases, Diabetes type I.

Vasculites are from medium to small vessel vasculitis or large vessel vasculitis, arthritides are selected from seronegative and seropositive rheumatoid arthritis, psoriatic arthritis, Bechterew's disease, juvenile idiopathic arthritis; inflammatory bowl diseases are selected from Crohn's disease or ulcerative colitis; diseases of the liver and the bile duct are selected from autoimmuno-hepatitis, primary biliary cirrhosis, primary sclerosing Cholangitis; autoimmune diseases of the thyroid gland are selected from Hashimoto's thyreoiditis, Grave's disease; autoimmune diseases of the connective tissue are selected from systemic lupus erythematosus disease, Sjörgen's syndrome, scleroderma, dermato- and poly-myositis, Sharp syndrome, systemic sclerosis and CREST syndrome; neurologic autoimmune diseases are selected from multiple sclerosis, chronic inflammatory demyelating polyneuropathy (CIDP), myasthenia gravis. Medium to small vasculitis is selected from classical panarteritis nodosa, granulomatosis with polyangiitis, microscopic panarteritis, Churg-Strauss syndrome Behcet's disease and the large vessel vasculitis is selected from giant cell arteritis, polymyalgia rheumatica, Takayasu's arteritis.

The progranulin detected and/or quantifies according to the method of the invention is hyper-phosphorylated. Preferably the amino acid sequence of progranulin is as set forth in SEQ ID NO: 1 or a fragment or variant thereof. The hyper-phosphorylated progranulin detected preferably comprises or consists of the amino acid sequence SEQ ID NO: 2 or fragment thereof. Preferably, the detected progranulin or fragment thereof is hyper-phosphorylated at a serine or at at least one serine. Preferably, the hyper-phosphorylated serine is the serine corresponding to S81 position or S82 position of SEQ ID NO: 1. Preferably, the hyper-phosphorylated progranulin is phosphorylated at the serine corresponding to S81.

Preferably, the progranulin comprises a hyper-phosphorylated serine, preferably the serine corresponding to S81 of SEQ ID NO: 1.

Hence, in an aspect the present invention is directed to hyper-phosphorylated progranulin or hyper-phosphorylated antigenic peptide/fragment thereof.

Preferably, the hyper-phosphorylated progranulin or the hyper-phosphorylated antigenic peptide/fragment or variant thereof has the amino acid sequence as set forth in SEQ ID NO: 1 or a fragment or a variant thereof. The hyper-phosphorylated progranulin or the hyper-phosphorylated antigenic peptide/fragment or variant thereof preferably comprises or consists of the amino acid sequence SEQ ID NO: 2. Preferably, the hyper-phosphorylated progranulin or the hyper-phosphorylated antigenic peptide/fragment or variant thereof is hyper-phosphorylated at a serine or at at least one serine. Preferably, the hyper-phosphorylated serine is the serine corresponding to S81 position or S82 position of SEQ ID NO: 1. Preferably, the serine corresponding to S81 is hyper-phosphorylated. Preferably, SEQ ID NO: 2 contains the serine corresponding to S81 hyper-phosphorylated.

Preferably, the progranulin comprises a hyper-phosphorylated serine, preferably the serine corresponding to S81 of SEQ ID NO: 1.

C) Progranulin Aberrant Conversion Pathway and its Detection

In another aspect, the present invention is directed to an in vitro method for diagnosing an autoimmune disease in a subject suspected of having an autoimmune disease comprising determining an aberrant conversion pattern of progranulin into granulins wherein the presence of the aberrant conversion pattern is indicative of an autoimmune disease. The aberrant pattern is for example as disclosed in FIG. 16 b) wherein the aberrant cleaving products of 55 kDa and 25KDa are disclosed

As already explained above, the present inventors have identified an aberrant conversion pattern of progranulin into granulins in patients affected by an autoimmune disease (FIG. 16). Preferably, the aberrant pattern is present in an anti-progranulin autoantibodies positive subject according to the present invention. Preferably, the aberrant pattern is present in “a hyper-phosphorylated progranulin or fragment thereof positive subject” according to the present invention. Preferably, the aberrant pattern is present in a subject positive to the hyper-phosphorylated progranulin or fragment thereof according to the present invention and positive to the anti-progranulin autoantibodies according to the present invention.

Preferably, the aberrant pattern is characterized by one or more aberrant progranulin cleaving products. Thus, a subject may have an aberrantly processed form of progranulin. Particularly, one progranulin aberrant cleaving product has an apparent molecular weight of 55 kDa±5 kDa as is determinable by SDS PAGE or the like as described herein. This cleaving product is preferably a N-terminal fragment of progranulin. This cleaving product is preferably hyper-phosphorylated. One progranulin cleaving product may comprise or consist of granulins G, F, B, A and C. This cleaving product is preferably hyper-phosphorylated. Preferably it is hyper-phosphorylated at the serine corresponding to S81 of SEQ ID NO 1. This cleaving product is an N-terminal fragment of progranulin. The progranulin cleaving product having an apparent molecular weight of 55 kDa±5 kDa as is determinable by SDS PAGE and the progranulin cleaving product comprising or consisting of granulins G, F, B, A and C may be the same cleaving product (see for example FIG. 16).

Another progranulin cleaving product is a cleaving product with apparent molecular weight of 25 kDa±5 kDa as is determinable by SDS PAGE or the like as disclosed herein. The progranulin cleaving is a C-terminal fragment of progranulin. One progranulin cleaving product may comprise or consist of granulins D and E. The cleaving product is a C-terminal fragment of progranulin. The progranulin cleaving product having an apparent molecular weight of 25 kDa±5 kDa as is determinable by SDS PAGE or the like and the progranulin cleaving product comprising or consisting of granulins D and E may be the same s (see for example FIG. 16).

Preferably, the aberrant pattern is characterized by a progranulin cleaving product. This cleaving product has an apparent molecular weight of 55 kDa±5 kDa as is determinable by SDS PAGE or the like. The cleaving product is an N-terminal fragment of progranulin. The cleaving product is preferably hyper-phosphorylated. This cleaving product contains a serine corresponding to S81 of SEQ ID NO 1. Preferably, the serine is hyper-phosphorylated.

Hence, the method according to the present invention comprises determining the presence of a progranulin cleaving product, wherein this cleaving product has an apparent molecular weight of 55 kDa±5 kDa as is determinable as described herein, wherein the presence of the cleaving product is indicative of an aberrant conversion pattern. Optionally, the method comprises determining the presence of a progranulin isoform wherein this cleaving product has an apparent molecular weight of 25 kDa±5 kDa as is determinable as described herein, wherein the presence of the cleaving product is indicative of an aberrant conversion pathway. Preferably, the cleaving product with an apparent molecular weight of 55 kDa±5 kDa as is determinable as described herein is a N-terminal fragment of progranulin. Preferably, the cleaving product with an apparent molecular weight of 25 kDa±5 kDa as is determinable as described herein is a C-terminal fragment of progranulin.

Alternatively, the method comprises determining the presence of a or at least a progranulin aberrant cleaving product wherein the aberrant cleaving product comprises or consists of granulins G, F, B, A and C wherein the presence of this cleaving product is indicative of an aberrant conversion pathway. This cleaving product is preferably a hyper-phosphorylated fragment of progranulin. This cleaving product preferably contains a serine corresponding to S81 of SEQ ID NO: 1. Preferably, the serine is hyper-phosphorylated. Alternatively or additionally, the method comprises determining the presence of a progranulin aberrant cleaving product wherein the cleaving product comprises or consists of granulins D and E wherein the presence of the cleaving product is indicative of an aberrant conversion pattern of progranulin.

Alternatively, the method comprises determining the presence of a progranulin aberrant cleaving product wherein the cleaving product comprises granulins G, F, B, A and C which has an apparent molecular weight of 55 kDa±5 kDa as is determinable as described herein, wherein the presence of the cleaving product is indicative of an aberrant conversion pattern. Alternatively or additionally, the method comprises determining the presence of a progranulin aberrant cleaving product wherein the cleaving product comprises granulins D and E which has an apparent molecular weight of 25 kDa±5 kDa as is determinable as described herein, wherein the presence of the cleaving product is indicative of an aberrant conversion pattern.

Hence, the present invention is directed to an in vitro method for diagnosing an autoimmune disease in a subject suspected of having the autoimmune disease comprising
a) analyzing/assaying a sample from the subject to detect the presence of a progranulin aberrant cleaving product wherein this cleaving product has an apparent molecular weight of 55 kDa±5 kDa as is determinable as described herein in particular as determinable with SDS PAGE, and/or
b) analyzing/assaying a sample from the subject to detect the presence of a progranulin aberrant cleaving product wherein this cleaving product has an apparent molecular weight of 25 kDa±5 kDa as is determinable as described herein in particular as determinable with SDS PAGE, and/or
b) correlating the presence of said aberrant cleaving product(s) with an autoimmune disease wherein the presence of said cleaving product(s) is indicative of an autoimmune disease in the subject.

Preferably, the aberrant cleaving product having apparent molecular weight of 55 kDa±5 kDa comprises a hyper-phosphorylated serine, preferably at least a hyper-phosphorylated serine. Preferably, the hyper-phosphorylated serine is the serine corresponding to position S81 or S82 in SEQ ID NO: 1. Preferably, the hyper-phosphorylated serine is the serine corresponding to position S81 in SEQ ID NO: 1.

Hence, the present invention is directed to an in vitro method for diagnosing an autoimmune disease in a subject suspected of having the autoimmune disease comprising
a) analyzing/assaying a sample from the subject to detect the presence of a progranulin aberrant cleaving product wherein the cleaving product comprises or consists of granulins G, F, B, A and C, and/or
b) analyzing a/the sample from the subject to detect the presence of a progranulin aberrant cleaving product wherein the cleaving product comprises or consists of granulins D and E, and/or
c) correlating the presence of said cleaving product(s) with an autoimmune disease,
   wherein the presence of said cleaving product is indicative of an autoimmune disease in the subject.

Preferably, the cleaving product which comprises or consists of granulins G, F, B, A and C comprises a hyper-phosphorylated serine, preferably at least a hyper-phosphorylated serine. Preferably, the hyper-phosphorylated serine is the serine corresponding to position S81 or S82 in SEQ ID NO: 1. More preferably, the hyper-phosphorylated serine is the serine corresponding to position S81 in SEQ ID NO: 1.

Hence, the present invention is directed to a in vitro method for diagnosing an autoimmune disease in a subject suspected of having the autoimmune disease comprising
a) analyzing/assaying a sample from the subject to detect the presence of a aberrant progranulin cleaving product wherein this cleaving product has an apparent molecular weight of 55 kDa±5 kDa as is determinable as described herein and comprises or consists of granulins G, F, B, A and; and/or
b) analyzing/assaying a sample from the subject to detect the presence of a progranulin cleaving product wherein this cleaving product has an apparent molecular weight of 25 kDa±5 kDa as is determinable as described herein and comprises or consists of granulins D and E,
c) correlating the presence of said cleaving product(s) with an autoimmune disease,
   wherein the presence of said cleaving product is indicative of an autoimmune disease in the subject.

Preferably, the cleaving product of point a) comprises a hyper-phosphorylated serine, preferably at least a hyper-phosphorylated serine. Preferably, the hyper-phosphorylated serine is the serine corresponding to position S81 or S82 in SEQ ID NO: 1. More preferably, the hyper-phosphorylated serine is the serine corresponding to position S81 in SEQ ID NO: 1.

In an aspect, the present invention is directed to a method for diagnosing an autoimmune disease in a subject suspected of having the autoimmune disease comprising
a) performing an electrophoresis on a sample from the subject to detect an aberrant conversion pattern of progranulin into granulin;
b) obtain an electrophoretic conversion pattern of progranulin;
c) comparing the obtained electrophoretic pattern with the electrophoretic pattern of the normal conversion pattern of progranulin into granulin to determine the presence, in obtained electrophoretic pattern, of at least a progranulin aberrant cleaving product wherein said at least cleaving product has an apparent molecular weight of 55 kDa ±5 kDa as is determinable as described herein or said at least cleaving product has an apparent molecular weight of 25 kDa±5 kDa as is determinable as described herein,
   wherein the presence of said cleaving product(s) is indicative of an autoimmune disease in the subject.

Preferably, the cleaving product having an apparent molecular weight of 55 kDa±5 kDa comprises a hyper-phosphorylated serine, preferably at least a hyper-phosphorylated serine. Preferably, the hyper-phosphorylated serine is the serine corresponding to S81 or S82 in SEQ ID NO: 1. Preferably the hyper-phosphorylated serine is the serine corresponding to S81 in SEQ ID NO: 1.

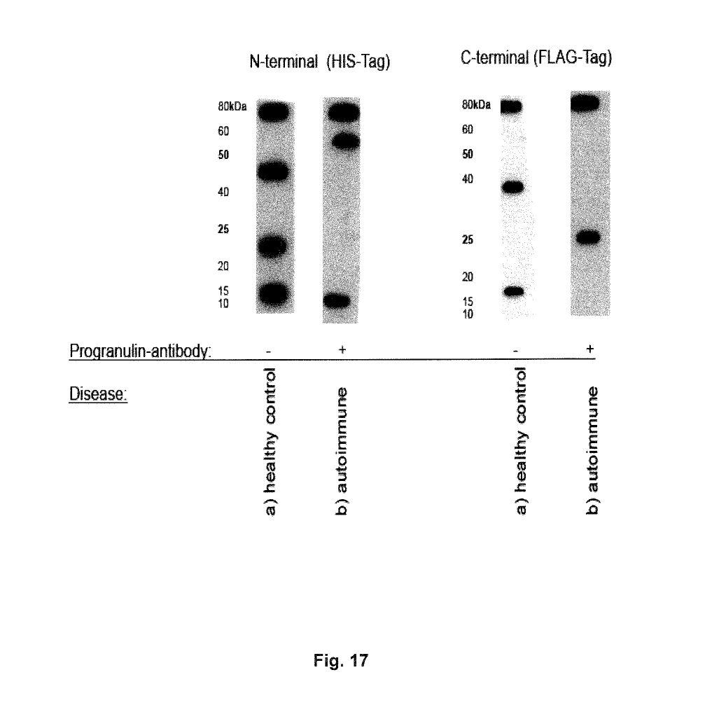

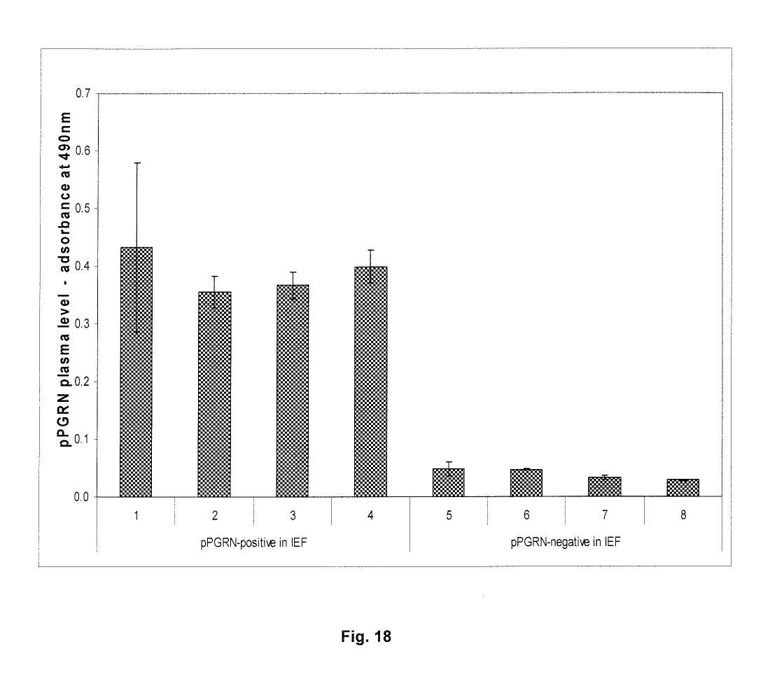

Preferably the electrophoresis is a SDS PAGE electrophoresis preferably a gradient SDS PAGE gel. More preferably, the electrophoresis is western blotting and the electrophoretic conversion pattern is a western blotting pattern. Examples of Western blotting conversion patterns are e.g. those of FIG. 17 wherein the Western blotting normal conversion pattern and the aberrant pattern are shown.

In an aspect, the present invention is directed to an in vitro method for diagnosing an autoimmune disease in a subject suspected of having the autoimmune disease comprising
a) performing an electrophoresis on a biological sample from the subject to detect an aberrant conversion pattern of progranulin into granulin;
b) obtaining an electrophoretic conversion pattern
c) comparing the obtained electrophoretic pattern with the electrophoretic pattern of the normal conversion pattern of progranulin into granulin to determine the presence, in obtained electrophoretic pattern, of a progranulin aberrant cleaving product wherein the cleaving product comprises or consists of granulins G, F, B, A and C,
d) correlating the presence of said cleaving product with an autoimmune disease,
   wherein the presence of said cleaving product is indicative of an autoimmune disease in the subject.

Preferably, the cleaving product does not comprise granulin D and E. Preferably, the cleaving product comprises a hyper-phosphorylated serine, preferably at least a hyper-phosphorylated serine. Preferably, the hyper-phosphorylated serine is the serine corresponding to position S81 or S82 in SEQ ID NO: 1. Preferably, the hyper-phosphorylated serine is the serine corresponding to position 81 in SEQ ID NO: 1. Preferably, the cleaving product is a N-terminal cleaving product of progranulin.

Preferably the electrophoresis is a SDS PAGE electrophoresis preferably a gradient SDS PAGE gel. More preferably, the electrophoresis is western blotting and the electrophoretic conversion pattern is a western blotting pattern. Examples of Western blotting conversion patterns are e.g. those of FIG. 17 wherein the Western blotting normal conversion pattern and the aberrant pattern are shown.

In an aspect, the present invention is directed to a method for diagnosing an autoimmune disease in a subject suspected of having the autoimmune disease comprising
a) performing an electrophoresis on a biological sample from the subject to detect an aberrant conversion pattern of progranulin into granulin;
b) obtaining an electrophoretic conversion pattern
c) comparing the obtained electrophoretic pattern with the electrophoretic pattern of the normal conversion pattern of progranulin into granulin to determine the presence, in obtained electrophoretic pattern, of a progranulin cleaving product wherein said cleaving product has an apparent molecular weight of 55 kDa±5 kDa as is determinable as described herein and wherein the cleaving product comprises or consists of granulins G, F, B, A and C,
   wherein the presence of said cleaving product is indicative of an autoimmune disease in the subject.

Preferably, the cleaving product does not comprise granulin D and E.

Preferably, the cleaving product comprises a hyper-phosphorylated serine, preferably at least a hyper-phosphorylated serine. Preferably, the hyper-phosphorylated serine is the serine corresponding to position S81 or S82 in SEQ ID NO: 1. Preferably, the hyper-phosphorylated serine is the serine corresponding to S81 in SEQ ID NO: 1. Preferably, the cleaving product is a N-terminal cleaving product of progranulin.

Preferably the electrophoresis is a SDS PAGE electrophoresis preferably a gradient SDS PAGE gel. More preferably, the electrophoresis is western blotting and electrophoretic conversion pattern is a western blotting pattern. Examples of Western blotting conversion patterns are e.g. those of FIG. 17 wherein the Western blotting normal conversion pattern and the aberrant pattern are shown.

The biological sample used for the above disclosed methods of chapter C) “Progranulin aberrant conversion pathway and its detection” is from an individual suspected of having or at a risk of an autoimmune disease and the presence of hyper-phosphorylated progranulin or hyper-phosphorylated peptide/fragment thereof in the biological sample is indicative of an autoimmune disease. Any biological sample is contemplated by the present invention such as tissues or liquid sample. Preferred is a liquid sample such as serum, blood, plasma and/or cerebrospinal fluid. The biological sample is preferably from an individual suspected of having or at a risk of an autoimmune disease. The presence of anti-progranulin-autoantibodies in the biological sample is indicative of an autoimmune disease. Any biological sample is contemplated by the present invention such as tissues or liquid sample. Preferred is a liquid sample such as serum, blood, plasma and/or cerebrospinal fluid. Preferred are also whole blood cells, lysate cells in particular whole blood lysate cells.

An autoimmune disease can be detected according to the methods of the invention as herein disclosed in chapter C) “Progranulin aberrant conversion pathway and its detection”. The autoimmune diseases is from the group of vasculitis, arthritides, autoimmune diseases of the connective tissue, inflammatory bowel diseases, autoimmune diseases of the liver and the bile duct, autoimmune disease of the thyroid gland, dermatologic autoimmune diseases, neurologic immune diseases, Diabetes type I. Preferably is vasculitis and arthritides. More preferably is vasculitis.

Vasculites are selected from medium to small vessel vasculitis or large vessel vasculitis, arthritides are selected from seronegative and seropositive rheumatoid arthritis, psoriatic arthritis, Bechterew's disease, juvenile idiopathic arthritis; inflammatory bowl diseases are selected from Crohn's disease or ulcerative colitis; diseases of the liver and the bile duct are selected from autoimmuno-hepatitis, primary biliary cirrhosis, primary sclerosing Cholangitis; autoimmune diseases of the thyroid gland are selected from Hashimoto's thyreoiditis, Grave's disease; autoimmune diseases of the connective tissue are selected from systemic lupus erythematosus disease, Sjörgen's syndrome, scleroderma, dermato- and poly-myositis, Sharp syndrome, systemic sclerosis and CREST syndrome; neurologic autoimmune diseases are selected from multiple sclerosis, chronic inflammatory demyelating polyneuropathy (CIDP), myasthenia gravis. Medium to small vasculitis is selected from classical panarteritis nodosa, granulomatosis with polyangiitis, microscopic panarteritis, Churg-Strauss syndrome Behcet's disease and the large vessel vasculitis is selected from giant cell arteritis, polymyalgia rheumatica, Takayasu's arteritis.

D) Progranulin Antibodies

In an aspect the present invention is directed to an antibody or antigen-binding fragment thereof which binds to progranulin or to a progranulin antigenic peptide/fragment or variant thereof.

The antibody or antigen-binding fragment binds specifically to progranulin and/or hyper-phosphorylated progranulin and/or to a progranulin antigenic peptide/fragment and/or variant thereof and/or to a hyper-phosphorylated progranulin antigenic peptide/fragment or variant thereof.

The present invention it is further directed to antibody or antigen-binding fragment that binds specifically to hyper-phosphorylated progranulin and/or hyper-phosphorylated progranulin antigenic peptide/fragment or hyper-phosphorylated variant thereof, but do not bind to non-hyperphosphorylated progranulin. Preferably the antibody or antigen-binding fragment that binds specifically to hyper-phosphorylated progranulin and/or hyper-phosphorylated progranulin antigenic peptide/fragment or hyper-phosphorylated variant thereof, but does not bind to non-hyperphosphorylated progranulin or fragment or variant thereof, binds to hyper-phosphorylated progranulin and/or hyper-phosphorylated progranulin antigenic peptide/fragment or hyper-phosphorylated variant thereof which is hyperphosphorylated at the serine corresponding to S81 of SEQ ID NO 1.

Preferably, the antibody or antigen-binding fragment binds specifically to a region defined by SEQ ID NO: 2 of progranulin and/or to hyper-phosphorylated progranulin and/or to a progranulin antigenic peptide/fragment and/or variant thereof and/or to a hyper-phosphorylated progranulin antigenic peptide/fragment or variant thereof. Preferably, the antibody specifically binds to the region defined by SEQ ID NO: 2 which comprises a serine corresponding to S81 and S82 (of SEQ ID NO 1) which may be hyper-phosphorylated. Preferably, the antibody specifically binds to the region defined by SEQ ID NO: 2 wherein the serine corresponding to S81 is hyper-phosphorylated and does not bind to non hyperphosphorylated progranulin.

Hence, in an embodiment, the antibody or antigen-binding fragment thereof binds to the N-terminal 112 amino acids sequence of progranulin SEQ ID NO: 1. Hence, in an embodiment, the antibody or antigen-binding fragment thereof binds to the N-terminal 112 amino acids sequence of progranulin SEQ ID NO: 1 wherein the serine corresponding to S81 of SEQ ID NO 1 is phosphorylated.

Hence the present invention is preferably directed to a site specific antibody of antigen binding fragment thereof that specifically binds a human progranulin only when phosphorylated at the serine corresponding to position S81 of progranulin, in particular corresponding to position S81 of SEQ ID NO 1, wherein said antibody or antigen binding fragment thereof does not bind said human progranulin when not phosphorylated at said serine.

Hence the present invention is also directed to a site specific antibody of antigen binding fragment thereof that specifically binds a human progranulin only when not phosphorylated at the serine corresponding to position S81 of progranulin, in particular corresponding to position S81 of SEQ ID NO 1, wherein said antibody or antigen binding fragment thereof does not bind said human progranulin when phosphorylated at said serine.

Fabs specific for hyperphosphorylated progranulin are disclosed in the experimental section.

In the present invention, the use of an anti-phospho-serine antibody and an anti-progranulin antibody for the detection of hyper-phosphorylated progranulin in a biological sample is contemplated. Anti-phospho-serine antibodies are known in the art. A kit comprising an anti-phospho-serine antibody and an anti-progranulin antibody is also an embodiment of the present invention. The antibodies comprised by the kit may be labeled with a signal as described herein. The kit may also or in addition comprise a labeled progranulin as described herein.

E) Progranulin as a Medicament

The present invention is directed to progranulin or a fragment thereof or a variant thereof for use in a method of ameliorating, preventing or treating an autoimmune disorder in a subject.

The progranulin or the fragment thereof or the variant for use in a method of ameliorating, preventing or treating an autoimmune disorder in a subject preferably comprises a serine corresponding to S81 of SEQ ID NO 1, preferably the fragment comprises or consists of the N-terminal 120 amino acid region of progranulin or fragment thereof; preferably the N-terminal 112 amino acid region of progranulin or fragment thereof, the N-terminal 120 amino acid region of SEQ ID NO 1 or fragment thereof; preferably the N-terminal 112 amino acid region of SEQ ID NO 1, preferably SEQ ID NO 2 or fragment thereof. The fragments have a length of 10-100, 10-80; 10-70 10-60, 10-50, 10-40, 10-30 and 10-20 aminoacids and preferably contain a serine corresponding to S81 in SEQ ID NO 1. Preferably, the fragments have a length of 10-50, 10-40, 10-30 and 10-20 aminoacids. For example, progranulin fragment 70-91AA corresponding to SEQ ID NO 17 without BIOT.

WO2010/120374 which is herein incorporated by reference discloses TNF antagonist or an isolated peptide comprising one or more granulin unit and one or more linker unit of Granulin/epithelin precursor (GEP), including variants thereof, and capable of antagonizing TNF family member signaling. The isolated peptide comprising one or more granulin unit and one or more linker unit of human GEP of FIG. 23 of WO2010/120374 (which herein incorporated by reference) and capable of antagonizing TNF and TNF/TNFR signaling. The isolated peptide preferably comprises at least 1/2F, 1/2A, and 1/2C granulin units of GEP and linker units P3, P4 and P5 of GEP as set out in FIG. 23 and/or FIG. 24 WO2010/120374 which herein incorporated by reference. The isolated peptide comprising 1/2F-P3-P4-′/2A-P5-1/2C as set out in FIG. 24 (SEQ ID NO: 2) WO2010/120374 which herein incorporated by reference. The isolated peptide is capable of binding TNFR1 and/or TNFR2. WO2010/120374 discloses a composition comprising one or more peptide(s) as disclosed in this paragraph and one or more of an anti-inflammatory agent or compound, an anti-cancer agent or compound, and an immunomodulatory agent. The composition is a therapeutic or pharmaceutical composition, optionally further comprising a pharmaceutically acceptable carrier, vehicle, or diluent. These peptides are useful in a method of modulating, blocking, or inhibiting TNF activity or signaling in a mammalian cell or subject comprising administering to said cell or subject. The preferred peptide is atsttrin. The methods are more specifically for preventing, alleviating, and/or treating a disease or condition mediated by TNF/TNFR signaling in a subject comprising administering to said subject the peptide. Said disease or condition is an inflammatory disease or condition, an immunological disease or condition, or cancer; said inflammatory disease or condition is selected from rheumatoid arthritis, osteoarthritis, ankylosing spondylitis, psoriasis, inflammatory bowel diseases, Crohn's disease, ulcerative colitis, uveitis, inflammatory lung diseases, and chronic obstructive pulmonary disease. The peptide of WO2010/120374 can be used as immuno modulator according to the present invention. They can be used as TNF blockers

In an embodiment of the invention the present progranulin or fragment (i.e. comprising a serine corresponding to S81 of SEQ ID NO 1, preferably the fragment comprising or consisting of the N-terminal 120 amino acid region of progranulin or fragment thereof; preferably the N-terminal 112 amino acid region of progranulin or fragment thereof, the N-terminal 120 amino acid region of SEQ ID NO 1 or fragment thereof; preferably the N-terminal 112 amino acid region of SEQ ID NO 1, preferably SEQ ID NO 2 or fragment thereof and preferably having a length of 10-100, 10-80; 10-60, 10-50, 10-40, 10-30, 10-20 aminoacids, preferably comprising a serine corresponding to S81 of SEQ ID NO 1 are administered in combination with atsttrin or with an immuno modulator as disclosed below for the treatment of autoimmune diseases according to the invention.

Preferably, the subject to be treated with the progranulin of fragment thereof according to the invention, has been found to be positive to at least one of the methods of the invention. Hence, preferably the subject is positive to the anti-progranulin autoantibody and/or is positive to hyper-phosphorylated progranulin and/or has an aberrant conversion pattern of progranulin into granulins all as disclosed in the present application. In the alternative to progranulin, an antibody against the subject's autoantibodies against progranulin may be used in a method of ameliorating, preventing or treating an autoimmune disorder. Such an antibody may be an anti-idiotypic antibody.

In another aspect, an immune modulator for use in a method of ameliorating, preventing or treating an autoimmune disorder in a subject, said subject having been diagnosed to comprise anti-progranulin autoantibodies. Immune modulator is for example a TNF-α-Blocker. TNF-α-Blocker are for example those mentioned in WO2012/120374, in particular Atsttrin.

The autoimmune disease to be treated is the group of vasculitis, arthritides, autoimmune diseases of the connective tissue, inflammatory bowel diseases, autoimmune diseases of the liver and the bile duct, autoimmune disease of the thyroid gland, dermatologic autoimmune diseases, neurologic immune diseases, Diabetes type I. Preferably is vasculitis and arthritides.

Vasculites are selected from medium to small vessel vasculitis or large vessel vasculitis, arthritides are selected from seronegative and seropositive rheumatoid arthritis, psoriatic arthritis, Bechterew's disease, reactive arthritis, juvenile idiopathic arthritis, enteropathic arthritis; inflammatory bowl diseases are selected from Crohn's disease or ulcerative colitis; diseases of the liver and the bile duct are selected from autoimmuno-hepatitis, primary biliary cirrhosis, primary sclerosing Cholangitis; autoimmune diseases of the thyroid gland are selected from Hashimoto's thyreoiditis, Grave's disease; autoimmune diseases of the connective tissue are selected from systemic lupus erythematosus disease, Sjörgen's syndrome, scleroderma, dermato- and poly-myositis, Sharp syndrome, systemic sclerosis and CREST syndrome; neurologic autoimmune diseases are selected from multiple sclerosis, chronic inflammatory demyelating polyneuropathy (CIDP), myasthenia gravis. Medium to small vasculitis is selected from classical panarteritis nodosa, granulomatosis with polyangiitis, microscopic panarteritis, Churg-Strauss syndrome Behcet's disease and the large vessel vasculitis is selected from giant cell arteritis, polymyalgia rheumatica, Takayasu's arteritis.

### Examples

### I. Anti-Progranulin-Autoantibodies and Progranulin Plasma Levels in Patients' Sera

Antigenic Targets by ELISA

Antigenic targets detected by screening the macroarray were confirmed as described before by ELISA Antigenic targets detected by screening the macroarray were confirmed as described before by ELISA (PREUSS K D, PFREUNDSCHUH M, AHLGRIMM M et al. A frequent target of paraproteins in the sera of patients with multiple myeloma and MGUS. Int J Cancer 2009; 125:656-661). The protein clones detected on the macroarray were obtained from the manufacturer (Bioscience, Dublin, Ireland) and recombinantly expressed with a C-terminal FLAG tag in HEK293 cells under the control of a CMV promoter (pSFI). Total cell extract was prepared and bound to Nunc maxisorb plates pre-coated overnight at 4° C. with anti-FLAG mAb at a dilution of 1:2,500 (v/v; Sigma, Munich). Blocking was performed with 1.5% (w/v) gelatin in TBS and washing steps were performed with TBS-Tx [TBS, 0.1% (v/v) Tx100]. The individual sera were diluted 1:100. ELISA was performed according to standard protocols with biotinylated goat anti-human IgG (heavy and light chain) at a dilution of 1:2,500 (Dianova) or subclass-specific sheep anti-human IgG1, IgG2, IgG3 and IgG4 (Binding Site, Birmingham, United Kingdom) at dilutions of 1:5,000 or rabbit anti human IgM (Dianova) at a dilution of 1:2,500. Following to this, correspondent biotinylated secondary antibodies were used for the immunoassays for IgG subclasses and IgM. Peroxidase-labeled streptavidin (Roche) was used at a dilution of 1:50,000.

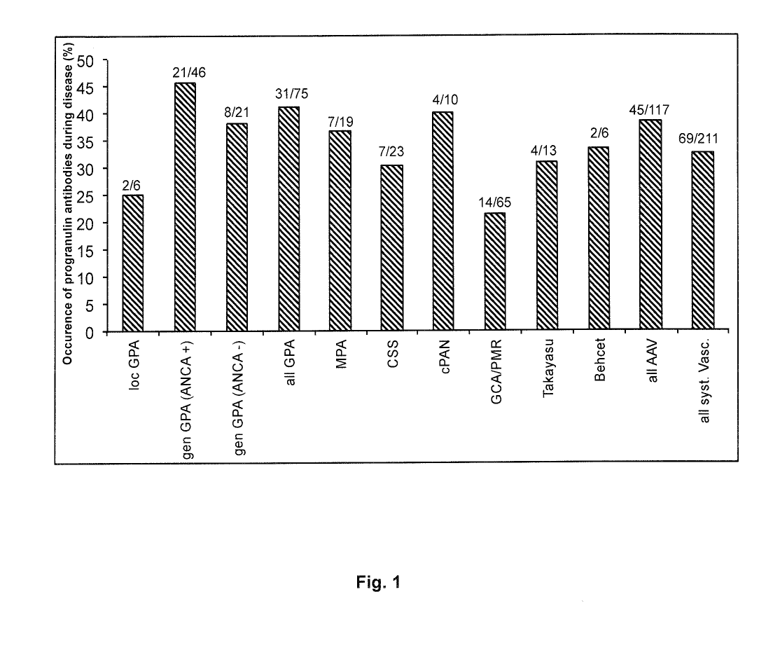

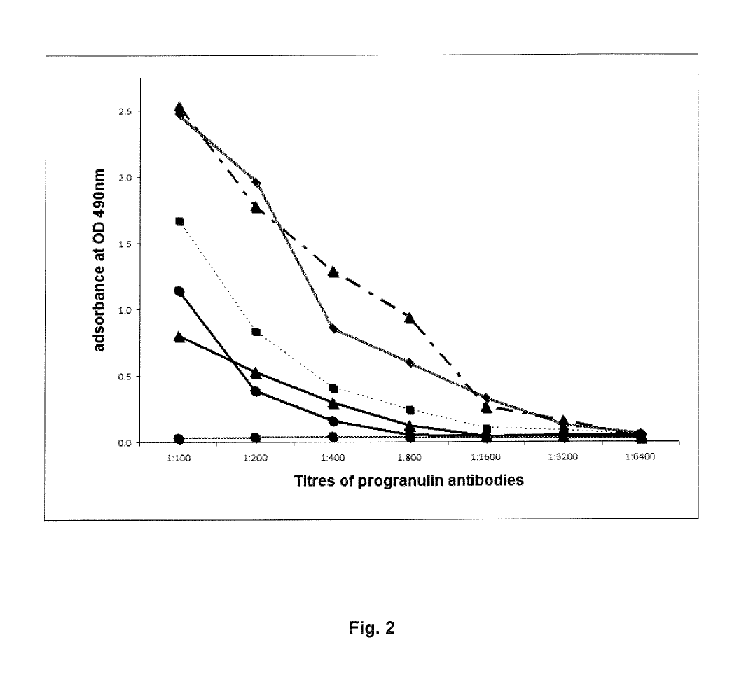

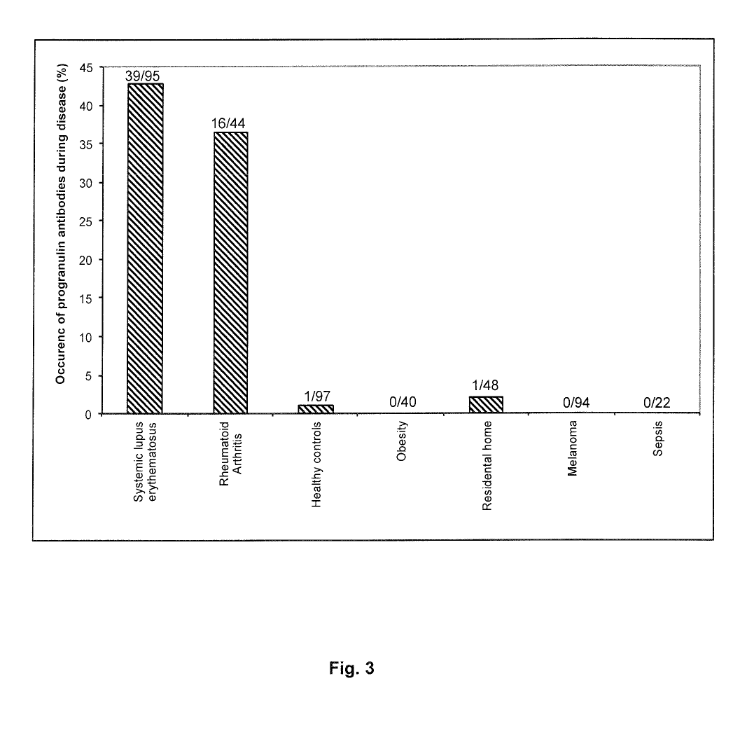

The anti-progranulin ELISA showed frequent occurrence of progranulin autoantibodies in the individual patient's sera, which were prior to this pooled on the macroarray (FIG. 1). ELISAs with expanded numbers of vasculitis patients confirmed the relatively high frequency of progranulin autoantibodies in the respective diseases. 14 of 65 (17%) patients with giant-cell arteritis and/or polymyalgia rheumatica, in 4 of 13 (27%) patients with Takayasu's arteriitis, in 4 of 13 (40%) patients with classical panarteritis nodosa and in 2 of 6 (33%) with Behcet's disease, 31 of 75 (41%) patients with granulomatosis with polyangiitis, 7 of 23 (30%) patients with Churg-Strauss syndrome and 7 of 19 (36%) were positive to anti progranulin autoantibodies. In an extended screening anti progranulin antibodies were also detected in systemic lupus erythematosus (39/95 or 40%) and rheumatoid arthritis (16/44 or 36%) (FIG. 3). There was only one case with a low-titer progranulin autoantibody reaction in the serum of 97 healthy controls. Furthermore, anti progranulin autoantibodies were found in 0/40 patients with obesity (0/40), in 1/48 residents of nursing homes, in 0/19 patients with sepsis and 0/94 patients with melanoma (FIG. 3). The control group of residents of nursing homes was chosen, because of the known prevalence of autoantibodies such as antinuclear antibodies and autoimmune disorders in the elderly population. Anti progranulin-autoantibody positive patients had titers ranging from 1:400 up to 1:3200 (FIG. 2).

Thirty-two progranulin antibody-positive vasculitis patients were tested for the IgG subclass of their progranulin antibodies. In 31/32 (96.9%) of them the progranulin antibodies belonged to the IgG1 subclass, and in 1/32 (3.1%) the progranulin antibodies belonged to the IgG2 subclass.

Identification of the Antibody-Binding Epitope of Progranulin

For the determination of the antibody-binding region of progranulin, recombinant progranulin fragments of different length were constructed with a C-terminal FLAG-tag and expressed in HEK293 cells under the control of a CMV promoter. ELISA and readout were performed as described above. Patient's sera were diluted 1:500.

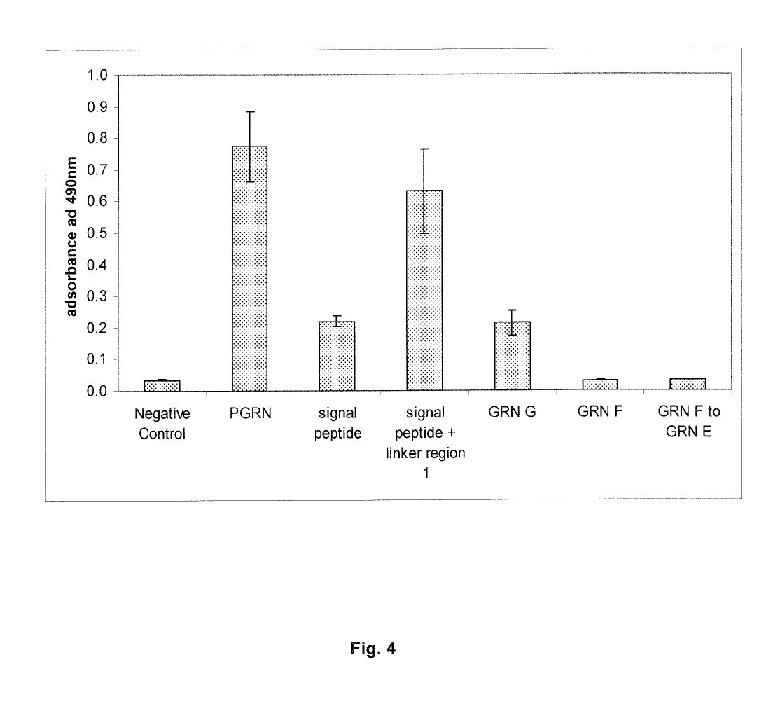

In particular, the progranulin clone identified by screening the macroarray with the pooled sera from patients with all 4 vasculitides was not a full-length clone, but consisted only of the N-terminal 250 aa. Therefore, ELISAs were performed with progranulin fragments of different lengths and with full-length progranulin. These ELISAs showed a strong binding of all anti-progranulin containing sera to fragments containing the N-terminal region (aa12-112) of progranulin, representing the signal peptide, the linker region and the first granulin motif (GRN G; FIG. 4). No binding with PGRN fragments starting from aa128, i.e. GRN F and following granulin motifs with their linker regions were observed.

Immunoassay for Antibodies Against Secretory Leukocyte Protease Inhibitor (SLPI) and Leukocyte Elastase Inhibitor (LEI)

The progranulin-stabilizing and thus anti-inflammatory protease inhibitors SLPI and LEI (also known as SERPINB1) were not represented on the macroarray. Therefore, ELISAs were established to screen sera for SLPI- and LEI-antibodies. The coding sequences of SLPI and LEI were amplified from a human lung tissue cDNA library by PCR using a proof-reading taq. Mutations were excluded by sequencing. Each of these two proteins was recombinantly expressed with a C-terminal FLAG tag in HEK293 cells under the control of a CMV promoter (pSFI). The immunoassays were performed as described above for the other antigens identified on the macroarray.

Progranulin ELISA Plasma Levels

Progranulin plasma levels were determined with a commercially available ELISA kit (AdipoGen, South Korea) according to the manufacturer's instructions.

The progranulin levels were significantly decreased in the plasma of progranulin antibody-positive vasculitis patients (n=7) compared to progranulin antibody-negative, but age-, sex-, and disease-matched vasculitis patients (n=7). The median progranulin plasma level observed in the sera from the healthy controls was set at 100%. Each of the progranulin antibody-positive patients had a lower progranulin plasma level than the individually matched progranulin antibody negative patient (Mann-Whitney test: p=0.002). When compared to healthy controls (n=8), the progranulin antibody-positive patients had also a significantly lower progranulin plasma level (Mann-Whitney test: p=0.001). There was no significant difference in the progranulin plasma levels of progranulin antibody-negative vasculitis patients compared to healthy controls (Mann-Whitney test: p=1.0). Similarly to progranulin plasma levels in vasculitis, we found significantly decreased progranulin plasma levels in progranulin antibody-positive patients with systemic lupus erythematosus (n=7) compared to progranulin antibody negative patients with systemic lupus erythematosus (n=7) (Mann-Whitney test: p=0.001) and in progranulin antibody-positive patients with rheumatoid arthritis (n=7) compared to progranulin antibody-negative patients with rheumatoid arthritis (n=7) (Mann-Whitney test: p=0.001).

Equivalent amounts of plasma (diluted 1:200) from progranulin-antibody negative and progranulin-antibody positive patients with systemic vasculitis and from healthy controls were loaded and separated in a 10% SDS-PAGE, transferred to PVDF membrane using a transblot semidry transfer cell (Bio Rad). Transferred proteins were incubated with rabbit anti-human IgG-Fc antibody at a dilution of 1:5,000 (Dianova) followed by incubation with a goat HRP labeled anti-rabbit IgG antibody at a dilution of 1:3,000 (Bio Rad). Then they were incubated with a HRP labeled rabbit anti-human progranulin antibody directed against the C-terminal part of progranulin (Bios) at a dilution of 1:1,000. ECL reagent (NEB) was utilized for immunoblot detection.

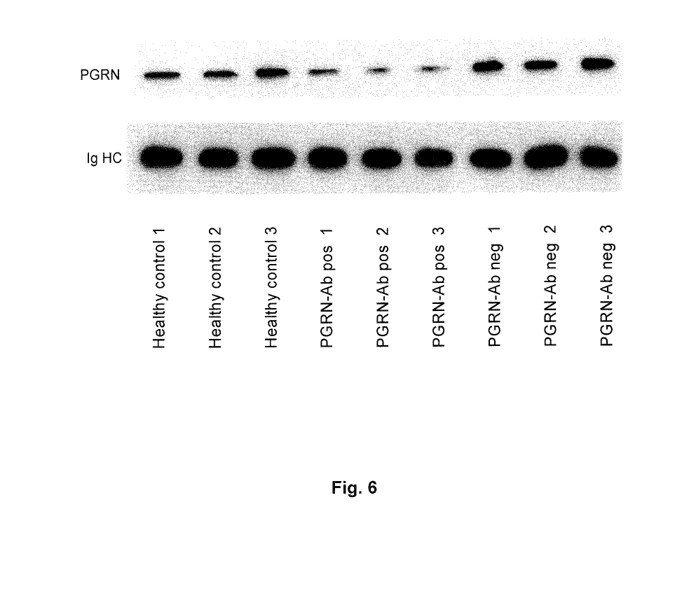

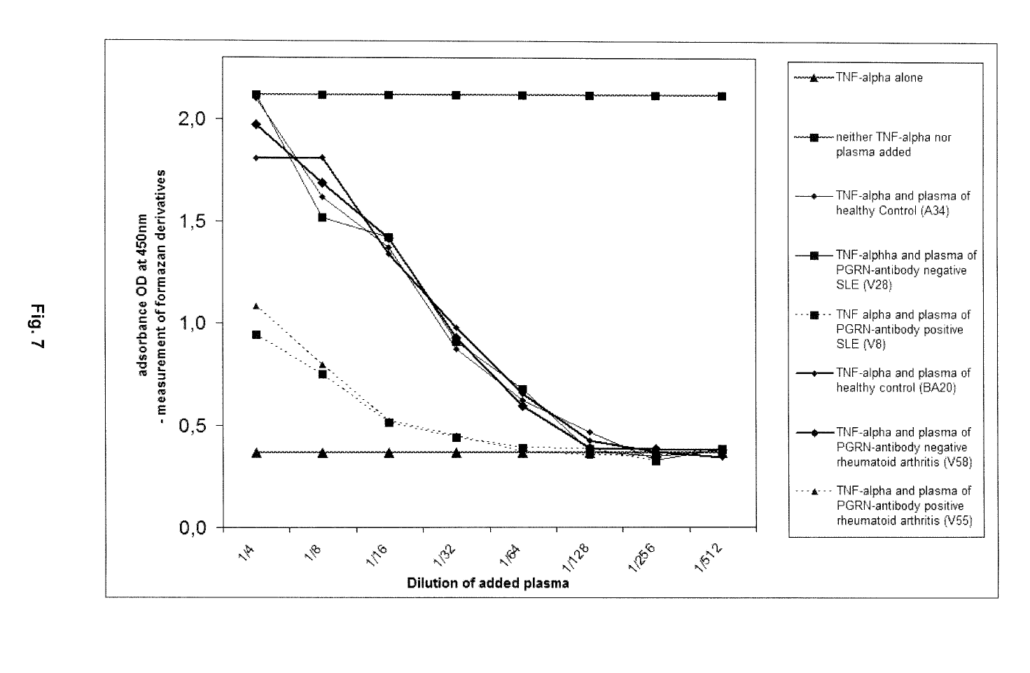

Western immunoblot analyses clearly showed decreased progranulin plasma levels in progranulin antibody-positive patients with vasculitis compared to progranulin antibody-negative patients with vasculitis or compared to healthy controls (FIG. 6).

For analyzing the differences in progranulin plasma levels between the groups, we used Mann-Whitney-Test when indicated. All statistical analyses were performed in SPSS Version 19.0 for Windows (IBM SPSS).

Association of Disease Activity and Progranulin-Antibody Status

We tested 68 blood samples obtained at different time and known disease activity status from 34 patients with granulomatosis with polyangiitis. From these 68 samples, 31 were obtained during active disease and 37 during remission. From the 31 obtained during active disease, 12 were positive for progranulin-antibodies. From 37 obtained in remission only 4 were positive for progranulin-antibodies. Statistical analysis shows a strongly significant association of positive progranulin-antibody status and active disease in granulomatosis with polyangiitis (X²-test: p=0.008). No statistical significant association between progranulin-antibody-status and disease activity were seen in 36 blood samples obtained at different time from 18 patients with Churg-Strauss syndrome and with 10 blood samples of 10 different patients with microscopic polyangiitis, of which 5 were obtained during active disease and 5 in remission. Summing-up all these samples obtained at different points of time from 57 patients with a known disease activity status of the respective small-vessel vasculitis, resulted in a statistical significant association between a positive progranulin-antibody status and active disease (X²-test: p=0.033).

In summary, anti progranulin autoantibodies have been identified in the sera of patients with systemic vasculitides and other autoimmune diseases. anti-progranulin autoantibodies have been identified also in sera of seronegative arthritis.

The results indicate that antibodies against progranulin tend to be present in the course of disease of patients with medium- to small-vessel vasculitides (classical panarteritis nodosa: 4/10; granulomatosis with polyangiitis 29/71; microscopic panarteritis: 7/19; Churg-Strauss syndrome: 7/23) large-vessel vasculitides (giant-cell arteritis/polymyalgia rheumatica: 13/76; Takayasu's arteritis (3/11). Extended screenings showed that anti-progranulin-antibodies are detected also in systemic lupus erythematosus (39/91) and in rheumatoid arthritis (16/44). The prevalence in control groups without autoimmune diseases was low: one in 97 healthy controls, no one in 40 patients with obesity, one of 48 residents of nursing homes, and no patient with melanoma (0/98) or sepsis (0/19) was found to have progranulin-antibodies (FIG. 3). The data obtained so far clearly prove that anti-progranulin-autoantibodies are specific for ongoing autoimmune processes.

Additionally, of particular interest is the inventors' finding that anti-progranulin autoantibodies are significantly associated with lower progranulin plasma levels in systemic vasculitides, rheumatoid arthritis and systemic lupus erythematosus, suggesting that theses autoantibodies have a neutralizing effect on progranulin. Progranulin is a strong anti-inflammatory mediator acting by potent inhibition of TNFR 1&2 (TANG W, LU Y, TIAN Q Y et al. Science 2011; 332:478-484.) as shown in a collagen-induced arthritis and collagen antibody induced arthritis model in C57BL/6 mice (GRN^{-/-}) mice, where the progranulin deficiency resulted in a dramatically accelerated and aggravated arthritis. Further mice which express a human TNF alpha transgene crossed to GRN knockout mice (TNF-Tg/GRN^{-/-}) develop spontaneously arthritis, which responds by rapid clinical recovery and decreased plasma levels of cartilage oligomeric matrix protein (COMP) to the application of recombinant human progranulin or Atsttrin, a recombinant progranulin derivative, reassembled by three modified granulin motifs and their accompanying linker regions (TANG W, LU Y, TIAN Q Y et al. Science 2011; 332:478-484 and LAI Y, YU X P, ZHANG Y et al. Osteoarthritis Cartilage 2012; 20:854-862). Thus, autoantibodies that bind and neutralize progranulin could result in a decreased endogenous inhibition of TNFR 1&2, and as a consequence in an increase of pro-inflammatory signals in patients with these autoimmune diseases. This hypothesis is supported by the fact that a positive progranulin-antibody status was statistically strongly associated with active disease in granulomatosis with polyangiitis (paired samples, n=34; X²-test: p=0.008).

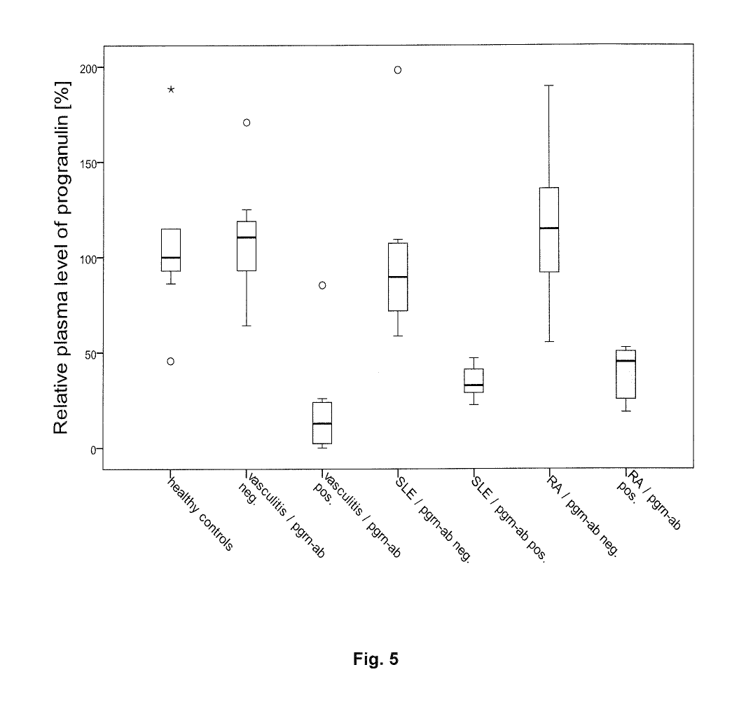

The present inventor has for the first time identified the spontaneous autoimmunity against progranulin. The epitope mapping identified an epitope within the region of the N-terminal 112 amino acids as the target of the anti-progranulin antibodies in all patients (FIG. 5). The highest frequencies of progranulin-antibodies were detected in small-vessel-vasculitides, systemic lupus erythematosus and rheumatoid arthritis (FIGS. 3 and 4). Not surprisingly, these diseases are characterized by prominent pathogenic roles of autoreactive B-lymphocytes with disease specific autoantibodies and the occurrence of ectopic lymphoid structures with B-cell clusters. But besides these autoimmune diseases with a typically predominant B-lymphocyte involvement, progranulin-antibodies were also detected in autoimmune diseases with a less established pathogenic role of B-lymphocytes such as classical panarteritis nodosa, Giant-cell arteritis or Takayasu's arteritis, even though there growing numbers of reports on beneficial effects of B-cell depletion (LAI Y, YU XP, ZHANG Y et al. Osteoarthritis Cartilage 2012; 20:854-862 and HOYER B F, MUMTAZ I M, LODDENKEMPER K et al. Ann Rheum Dis 2012; 71:75-79), the detection of increased numbers of plasma blasts correlating with active disease (HOYER B F, MUMTAZ I M, LODDENKEMPER K et al. Ann Rheum Dis 2012; 71:75-79) and the discovery of new autoantibodies, which support a pathogenic involvement of B lymphocyte in these diseases.

The identification of neutralizing progranulin autoantibodies in several different autoimmune diseases has clinical implications, because it suggests a new pro-inflammatory autoimmune mechanism common to subgroups of different autoimmune diseases.

Alternatively, the identification of autoimmune patients with progranulin autoantibodies could possibly guide individualized therapeutic strategies. Assuming that neutralization of secreted progranulin by its specific autoantibodies leads to excessive upregulation of the TNFR 1&2 dependent pathways, patients with anti-progranulin antibodies should presumably particularly profit from TNF-alpha blockers and also from Atsttrin.

### Cell Cytotoxicity Assay

Background of this cytotoxicity assay is the fact, that progranulin inhibits directly TNFR1 and 2 [1]. Furthermore the administration of progranulin had protected cells in vitro from cytotoxic effects of TNF-α (Tang et al., 2011). To test in vitro possible pathogenic effects of neutralizing progranulin-antibodies, plasma from progranulin-antibody positive patients and thus with obviously lower progranulin levels or plasma from progranulin-antibody negative patients or from healthy controls and thus with higher progranulin levels were administrated to cell cultures before the addition of TNF-α. Given the fact, that the half-life of progranulin is about 40 h [1] in contrast to the short half-life of TNF-α of about 20 min [6], which is typically for cytokines, the intrinsic amount of TNF-α in the administrated plasma could be neglected. Thus administration of plasma from a patient with neutralizing progranulin-autoantibodies to cells should theoretically lead to a decreased protection from the effects of the administrated TNF-α than the administration of plasma from a patient without neutralizing progranulin-autoantibodies. To detect such a possible influence of progranulin-antibodies on the sensitivity of cells to TNF-α, a non-radioactive cytotoxicity assay (EZ4U, Biomedica) was utilized according to the manufacturer's instructions. In short, in each well 8×103 WEHI-S mouse fibroblasts were seeded in 200 μl cell culture at 37° C. and at 5% CO2. The inhibitory functionality of human progranulin on murine TNF-receptors was shown in vitro and in vivo by Tang et al.[1]. To detect possible differences between added plasma of progranulin-antibody positive patients with progranulin-antibody negative patients and healthy controls, several test series were performed. Each test series comprised the addition of plasma from a progranulin-antibody positive and matched negative patient and of a healthy control in following dilutions 1:4, 1:8, 1:16, 1:32, 1:64, 1:128, 1:256, 1:512 and of TNF-α (100 pg/ml) to single wells cultured with WEHI-S cells. Therefore plasma samples of disease and therapy matched patients were chosen and plasma samples of patients receiving TNF-Blockers were excluded. Each plasma sample was pretreated by 1 mM CaCl2. As negative and positive controls served WEHI-S cells with neither addition of TNF-α nor plasma, or solely addition of 100 pg/ml TNF-α. After 24 h incubation at 37° C., 20 μl of chromophore substrate were added to each well. This chromophore substrate is only converted by vital cells. The absorbance of the product was measured at an OD of 450 nm.

The cytotoxic effect of TNF-α on the WEHI-S cells was significantly higher when plasma of progranulin antibody-positive patients was added compared to plasma of matched progranulin-antibody negative patients or to plasma from healthy controls. This difference was significant, even when the added plasma samples were diluted 1:64 (FIG. 7).

### II. Anti-Progranulin Autiantibodies and Progranulin Plasma Levels in Psoriatic Arthritis (PsA) Patients

Psoriatic arthritis (PsA) is a distinctive inflammatory arthritis that may develop in 5-40% of individuals suffering from psoriasis. Accordingly, the occurrence of PGRN-Abs in PsA was investigated.

### PGRN-Ab Levels

Therefore, PGRN-Abs were determined in 260 patients with PsA, 100 patients with psoriasis without arthritic manifestations (PsC) and 97 healthy controls using an ELISA. In short, the GRN gene, encoding progranulin (PGRN) was recombinantly expressed with a c-terminal FLAG-tag in HEK293 cells under the control of a CMV promoter (pSFI). Total cell extracts were prepared and bound to Nunc maxisorb plates pre-coated with murine anti-FLAG mAb at a dilution of 1:2,500 (v/v; Sigma, Munich) at 4° C. overnight. Blocking was performed with 1.5% (w/v) gelatine in TBS, and washing steps were performed with TBS-Tx. The individual sera were diluted 1:100. ELISA was performed according to standard protocols with biotinylated goat anti-human IgG (heavy and light chain) at a dilution of 1:2,500 (Dianova) or subclass-specific sheep anti-human IgG1, IgG2, IgG3 and IgG4 (Binding Site, Birmingham, United Kingdom) at dilutions of 1:5,000 or goat anti human IgM (Dianova) at a dilution of 1:2,500 or goat anti human IgA (Dianova) at a dilution of 1:2,500. Following to this, correspondent biotinylated secondary antibodies were used for the immunoassays for IgG subclasses and IgM. Peroxidase-labeled streptavidin (Roche) was used at a dilution of 1:50,000. As cutoff for positivity the average of the OD of the negative samples plus three standard deviations was applied.

### PGRN Plasma Levels

PGRN plasma levels were determined from subgroups by a commercially available ELISA-kit (AdipoGen, South Korea) according to the manufacturer's instructions. PGRN serum level of healthy controls was set at 100%.

### Cell Cytotoxicity

Possible functional effects of PGRN-antibodies were analysed in vitro by TNF-a mediated cytotoxicity assays using WEHI-S and HT1080 cells as described herein. A non-radioactive cytotoxicity assay (EZ4U, Biomedica) was performed according to the manufacturer's instructions. As target cells for this TNF-alpha induced cytotoxicity assay, the human colorectal adenocarcinoma HT29 cell-line was used. In short, 4×104 HT29 cells were seeded in 200 μl cell culture at 37° C. and at 5% CO2. To detect possible differences between added sera of patients with ulcerative colitis with and without PGRN-Abs or of patients with Crohn's disease with and without PGRN-Abs and of healthy controls, serum of a PGRN-Ab positive patients, serum of a matched PGRN-Ab-negative patients and serum of a healthy controls were administrated in dilutions from 1:4, to 1:512 to the cultured HT29 cells followed by the administration of TNF-a (10 μg/ml). Serum samples from gender-, age-, disease- and therapy modality-matched patients were chosen. Serum samples from patients receiving TNF-Blockers or other Biologicals were excluded. HT29 cells without addition of TNF-a and serum, or solely with addition of TNF-a (100 pg/ml) were used as positive and negative controls. After 24 h incubation at 37° C., 20 μl of chromophore substrate were added to each well. This chromophore substrate is only converted by vital cells. The adsorbance of the product was measured at an OD of 450 nm

PGRN-Abs were detected with relevant titres in 50/260 (19.23%) patients with PsA, but in 0/100 patients with psoriasis without arthritic manifestations (p=0.0001). All PGRN-Abs belonged to IgG. PGRN-Abs were significantly more frequent in PsA patients with enthesitis or dactylitis. PGRN-Abs were also more frequent in PsA patients receiving treatment with TNF-α-blockers than in patients treated without TNF-α-blockers (20.8 vs. 17.4%; p=0.016). PGRN plasma levels were significantly lower in PGRN-Ab-positive patients with PsA than in healthy controls and patients with psoriasis without arthritic manifestations (p<0.001), indicating a neutralizing effect of PGRN-Abs. Moreover cytotoxicity assays comparing PGRN-antibody positive with negative sera from matched patients with PsA, clearly showed a proinflammatory effect of progranulin-antibodies.

In conclusion, neutralizing PGRN-Abs occur with relevant titres in a subgroup of patients with PsA, but not in patients without arthritic manifestations (PsC). PGRN-Ab-positive patients had more frequently enthesitis or dactylitis. In TNF-a-induced cytotoxicity assays demonstrated, that the protective effects of progranulin were inhibited by serum containing PGRN-Abs. This suggests that PGRN-Ab might not only be useful as a diagnostic and prognostic marker, but may provide a proinflammatory environment in a subgroup of patients with PsA.

### III. Anti-Progranulin Autiantibodies in Inflammatory Bowel Disease (IBD) Patients

A possible occurrence of progranulin-antibodies in IBD and a possible pathogenic effect were investigated.

### PGRN Ab Levels

Therefore, sera samples of 141 patients with Crohn's disease and of 71 patients with ulcerative colitis were tested for progranulin-antibodies by ELISA as described in II. As cutoff for positivity, the average of the OD values of the negative samples plus three standard deviations was applied. From several patients more than one serum sample had been obtained during their course of disease. If at least one of these serum samples was positive for PGRN-Abs, this patient was regarded as positive for PGRN-Abs in the course of disease.

### PGRN Plasma Levels

PGRN plasma levels were detected by ELISA as described in II.

### Cell Cytotoxicity

Proinflammatory effects of PGRN-antibodies were analysed by TNF-α mediated cytotoxicity assays using HT29 cells according to the manufacturer's instructions (EZ4U, Biomedica) as described in II.

Detection of pANCA and cANCA

Detection of pANCA and cANCA had already been performed during clinical routine diagnostics according to standardized laboratory procedures by initial indirect immunofluorescence screening for perinuclear or cytoplasmic ANCA patterns, followed in positive or borderline cases by ELISA (Varelisa-Kit) for MPO-ANCA and Pr3-ANCA at the autoimmune diagnostic laboratory of the Department of Internal Medicine I at the Saarland University Medical School. The cut-off for positive MPO-ANCA and Pr3-ANCA was set according to the manufacturer's instructions. ASCA status had been determined in subgroups of patients with Crohn's disease and ulcerative colitis by a commercial laboratory.

Progranulin-antibodies were found in sera of 23/141 (16.31%) patients with Crohn's disease, and 15/71 (21.13%) patients with ulcerative colitis. Progranulin-antibodies belonged mostly to IgG1 (71.1%) and IgA (26.3%). They occurred in relevant titres and had significant neutralizing effects on progranulin serum levels. Moreover cytotoxicity assays comparing PGRN-antibody positive with negative sera from matched patients with IBD, showed a proinflammatory effect of progranulin-antibodies on HT29 cells. Progranulin-antibodies were more frequent than ANCAs in ulcerative colitis, but less frequent than ASCA in Crohn's disease.

In conclusion, progranulin-antibodies frequently occur in Chron's disease and ulcerative colitis. In TNF-α induced cytotoxicity assays using HT29 cells the protective effects of progranulin were inhibited by serum containing progranulin-antibodies, providing evidence for a proinflammatory effect of progranulin-antibodies in Crohn's disease and ulcerative colitis, by neutralization of the physiologic TNFR1 &2-inhibitor progranulin.

### IV: Hyperphosphorylated Progranulin

### Lymphoblastoid Cell Lines

Lymphoblastoid cell lines (LCLs) were established by infection of PBMCs with EBV as described before [2].

### Isoelectric Focusing

As no differences between progranulin-autoantibody positive and progranulin-autoantibody negative patients or healthy controls were found in the CDS and in the Western blot of full-length progranulin, isoelectric focusing was performed with whole blood cell lysates from progranulin-autoantibody positive and negative patients and from healthy controls.

Isoelectric focusing was performed as described before [3]. In short, whole blood cell extracts or extracts of LCLs were washed and treated with lysis buffer (8 M urea, 0.1 M NaH2PO4, 0.01 M Tris HCl, 0.1% NP40). Following to this, isoelectric focusing was performed (pH 3-10, Invitrogen) according to the manufacturer's protocol (1 hour 100 V, 1 hour 200 V, and 30 minutes 500 V). After semi-dry blotting on PVDF membranes (450 mA, 24V, 1 hour), patient's plasma was used for immunodetection of progranulin from whole blood cell lysates and murine anti-FLAG antibody (Sigma) was used at a dilution of 1:2,000 for immunodetection of progranulin from LCL lysates. These LCLs had been previously transfected with FLAG-tagged progranulin in pRTS. The membrane was blocked in TST/milk buffer (10% milk in 10 mM Tris/HCl, pH 7.5, 150 mM NaCl, 0.1% [v/v] Tween 20) overnight, washed, and incubated for 1 hour with plasma in TST or murine anti-FLAG-antibody. After 3 washings in TST, the membranes were incubated for 1 hour at room temperature with PDX-labeled goat anti-human IgG Ab (Dianova) diluted 1:5,000 or PDX-labeled anti-mouse IgG Ab (Biorad) in TST, subsequently washed in TST followed by ECL development. To perform IEF on different cell fractions, PBMCs and granulocytes were isolated by density double gradient centrifugation (Histopaque-1077 and -1119, Sigma). Monocytes, B-cells and T-cells were sorted with CD14, CD19 and CD3. Mucosa cells were obtained by swab from the oral cavities of patients and controls.

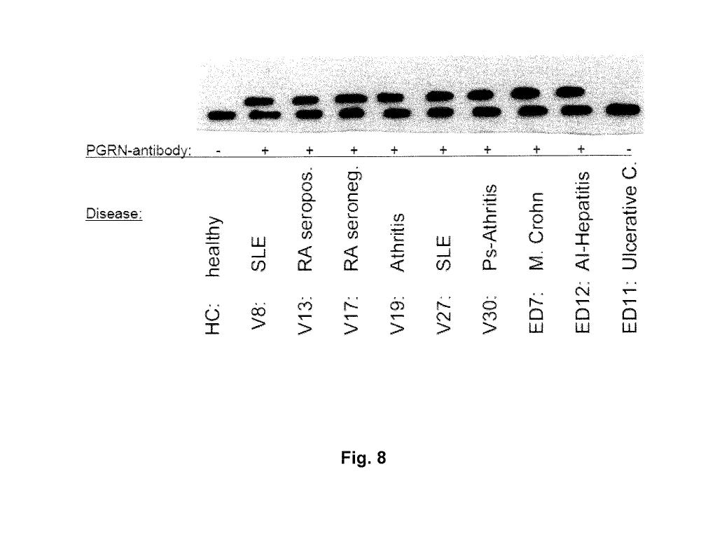

All of the progranulin-autoantibody positive patients and only them had a double band in the IEF of progranulin. This additional band showed a more negatively charged isoform of progranulin, suggesting an aberrant post-translational modification (FIG. 8).

Treatment with Alkaline Phosphatase

As there was no difference in molecular weight of the two isoforms detectable by western-blot analyses and the additional progranulin appeared to be charged more negatively, we analyzed progranulin for differences in the phosphorylation state.

Treatment of whole blood cell lysates or LCL lysates with alkaline phosphatase was conducted in principal as previously described [3]. FastAP thermo sensitive alkaline phosphatase (Fermentas) was used. In short LCLs were washed 3 times with PBS. The lysis was performed in a buffer of 10 mM Tris HCl pH 8 for 1 h at 4° C. Following to this the supplied FastAP-Buffer (10×Fast AP-Buffer: 100 mM Tris-HCl, pH 8.0, 50 mM MgCl2, 1 M KCl, 0.2% Triton X-100 and 1 mg/ml BSA) was added to the cell lysate. For 0.1 mg/ml lysed protein extract, 10 units of FastAP were utilized. After 1 h at 37° C., the alkaline phosphatase reaction was stopped by the addition of 50 mM EDTA. Equal volumes of sample and loading buffer were mixed, followed by IEF and immuno-detection.

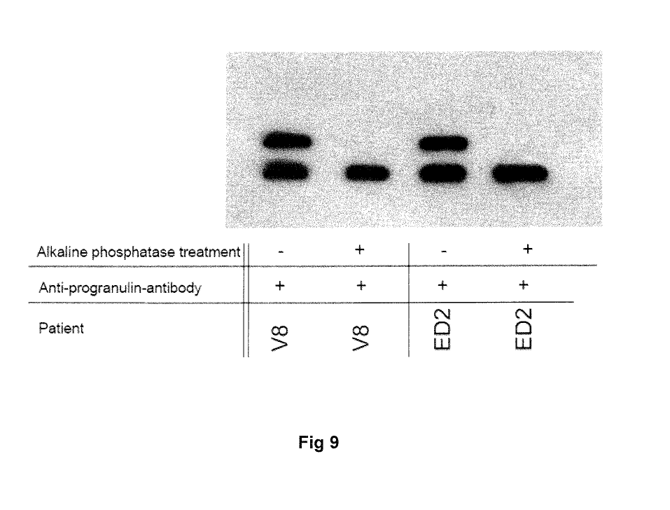

The pretreatment with alkaline phosphatase before IEF, led to a disappearance of the additional progranulin isoform (FIG. 9). This proved the existence/presence of an additionally hyper-phosphorylated progranulin isoform exclusively in progranulin-antibody positive patients.

### Progranulin Digestion

To study the conversion of hyper-phosphorylated and normal progranulin into mature granulins, whole blood cell lysates of patients with and without progranulin antibodies, i.e. with or without additionally hyper-phosphorylated progranulin were analyzed. Furthermore lysates of LCLs of progranulin-antibody positive patients and of controls, which had been transfected with pRTS-vector to express recombinant full-length progranulin or fragments of progranulin with N-terminal HIS- and Cterminal FLAG-tags were analyzed. The LCLs were washed 3 times with PBS followed by lysis in LS buffer (10 mM Tris HCl pH 8, 30 minutes 4° C.). The samples and controls were then submitted to western immunoblotting with SDS gradient-gels (9%-15%).

### Cloning of Progranulin Fragments and Expression in HEK293 Cells and LCLs

The progranulin fragments required for further experiments were constructed based on the coding sequence of full length progranulin, which had been amplified before from a human lung tissue cDNA library by PCR using a proof-reading taq. Mutations were excluded by sequencing.

Following primers were used:

PGRN-full length-EcoRV-s:
(SEQ ID NO: 4)
GAT ATC ATG TGG ACC CTG GTG AGC TGG.
PGRN-full length-EcoRV-as:
(SEQ ID NO: 5)
GAT ATC CAG CAG CTG TCT CAA GGC TGG
GranulinF-EcoRV-s:
(SEQ ID NO: 6)
GAT ATC ATG GAT AGT CAG TTC GAA TGC CCG
GranulinG-EcoRV-s:
(SEQ ID NO: 7)
GAT ATC ATG CAG GTT GAT GCC CAC TGC

Each of these progranulin fragments was recombinantly expressed with a c-terminal FLAG-tag and a n-terminal HIS-tag in HEK293 cells under the control of a CMV promoter (pSFI) or in LCLs in pRTS. Undirected insertions of progranulin fragments were performed via EcoRV

Following primers were used for this:

Granulin-His6-Start-EcoRV-s:
(SEQ ID NO: 8)
GAT ATC ATG CAT CAC CAT CAC CAT CAC ATG TGG ACC
CTG GTG AGC TGG
Granulin-His6-AA53-EcoRV-s:
(SEQ ID NO: 9)
GAT ATC ATG CAT CAC CAT CAC CAT CAC CTG AGC AGG
CAT CTG GGT
Granulin-His6-AA118-EcoRV-s:
(SEQ ID NO: 10)
GAT ATC ATG CAT CAC CAT CAC CAT CAC AAC AAC TCC
GTG GGT GCC
Granulin-His6-AA200-EcoRV-s:
(SEQ ID NO: 11)
GAT ATC ATG CAT CAC CAT CAC CAT CAC GTG GCC TTG
TCC AGC TCG
Granulin-AA260-EcoRV-as:
(SEQ ID NO: 12)
GAT ATC GCA CTT ACT CTG GAT CAG
Granulin-AA335-EcoRV-as:
(SEQ ID NO: 13)
GAT ATC ACA GGT ACC CTT CTG CGT
Granulin-AA417-EcoRV-as:
(SEQ ID NO: 14)
GAT ATC CTG ACA CTG CCC CTC AGC

### Site-Directed Mutagenesis

Using the QuickChange II Site-Directed Mutagenesis Kit (Stratagene) and a progranulin DNA fragment coding for FLAG-tagged 1 AA to 112 AA, mutants were constructed in which two serines were exchanged to alanines (Ser81Ala). These mutants were stably transfected into LCLs. Fragments of progranulin were amplified by PCR using the primers listed below and full-length progranulin as template followed by cloning into pSfi-FLAG and thereafter into pRTS as expression vector:

Following primers were used for site directed mutagenesis:

Granulin-AA81-mut-Ser-Ala-s:
(SEQ ID NO: 15)
ACC GTC TCA GGG ACT GCC AGT TGC TGC CC
Granulin-AA81-mut-Ser-Ala-as:
(SEQ ID NO: 16)
GGG CAG CAA CTG GCA GTC CCT GAG ACG GT

### IEF of Point Mutated Progranulin at Ser81Ala

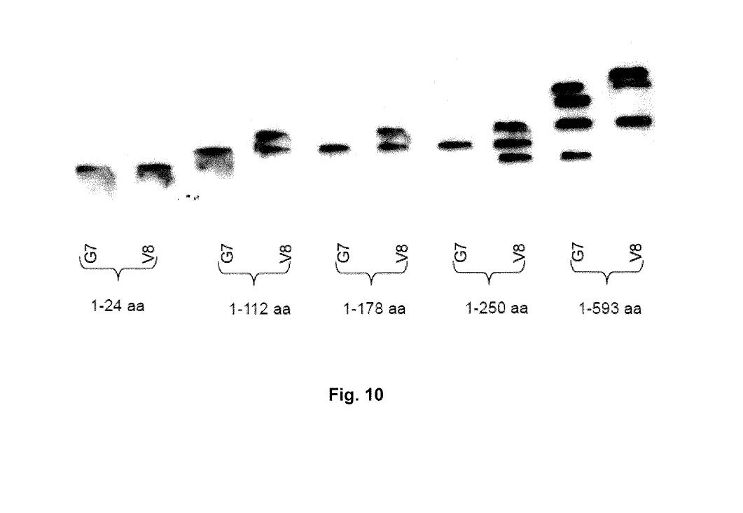

To narrow down the number of possible phosphorylation sites, we expressed progranulin-fragments with N-terminal FLAG-tags in a pRTS vector in LCLs of progranulin-antibody positive and progranulin-antibody negative patients. Following to this IEF was performed with the lysates of these LCLs and using anti-FLAG antibodies for immunodetection. By this means the localization of the phosphorylation site could be narrowed down to 24-112 AA (FIG. 10), which had been found to be the affinity region of all progranulin-autoantibodies. Ser81 appeared to have the highest probability scores as phosphorylation sites. So the CDS of progranulin was point mutated from Serine to Alanine (Ser81Ala) and these point mutated constructs were as well expressed in LCLs from progranulin-antibody positive and progranulin-antibody negative patients. Again IEF was performed with LCL lysates of these point mutated progranulin fragments. The additionally hyper-phosphorylated progranulin could not be detected anymore in Ser81Ala-progranulin Hence, Ser81 was identified as the hyper-phosphorylated amino acid.

### IEF of Progranulin in Different Cell Fractions

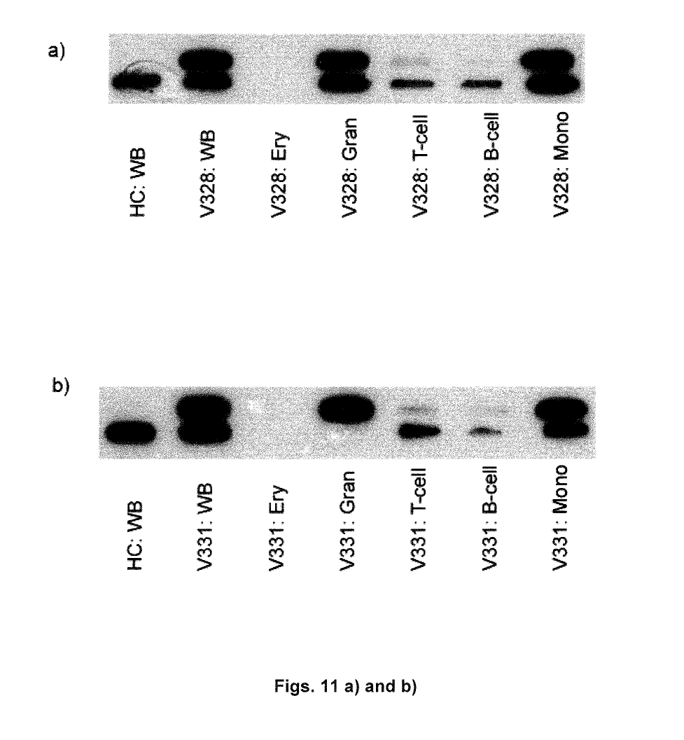

The hyper-phosphorylated progranulin isoform was strongly expressed in monocytes, granulocytes, weaker in B- and T-lymphocytes and not in erythrocytes (FIGS. 11 a) and 11 b)) and mucosa cells of the oral cavity (not shown).

IEF of Progranulin from Patients with Seroconversions of Progranulin-Antibody

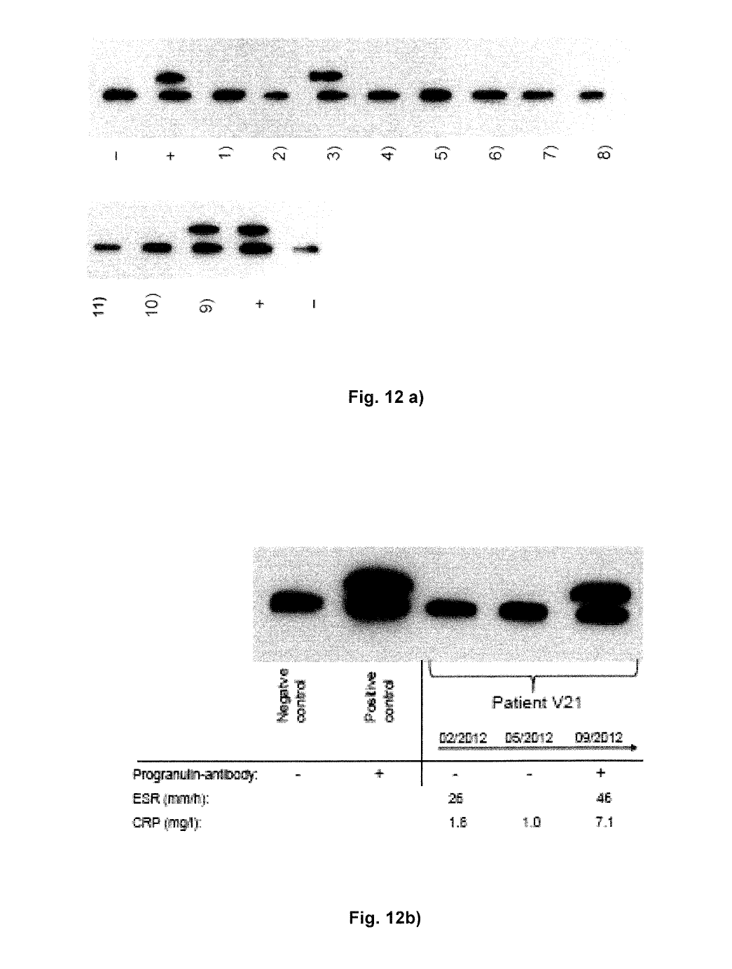

The IEFs were performed of whole blood cell lysates of eleven patients, who had previously all been progranulin-antibody positive and underwent seroconversions. No hyper-phosphorylated progranulin isoform was detectable in nine patients, who were progranulin-antibody negative at the time of IEF. In contrast the hyper-phosphorylated progranulin isoform was detectable in two patients, who were progranulin-antibody positive at the point of time when the IEF was performed (FIG. 12a). In analogy to this we observed in one patient, from which we had obtained serum and peripheral blood cell samples at three point of times within 10 months a gain of hyper-phosphorylated progranulin and a seroconversion of the progranulin-antibody status from negative to positive. Clinically this patient suffered from a flare of her autoimmune disease at the end of the 10 month period with elevated inflammatory parameters like CRP, ESR, and decreased complement C3 and C4 (FIG. 12b).

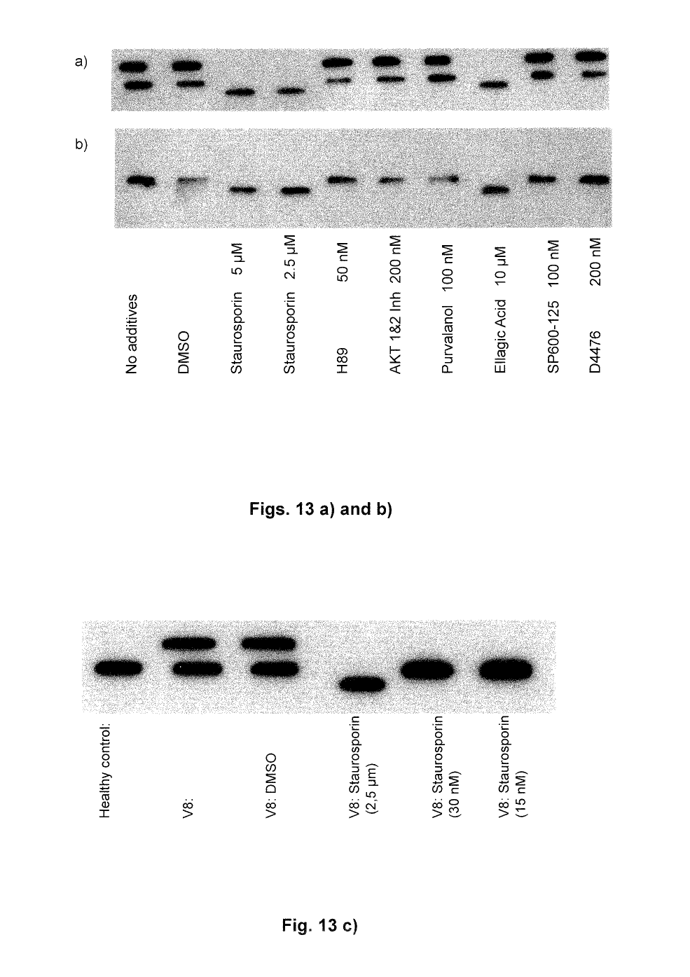

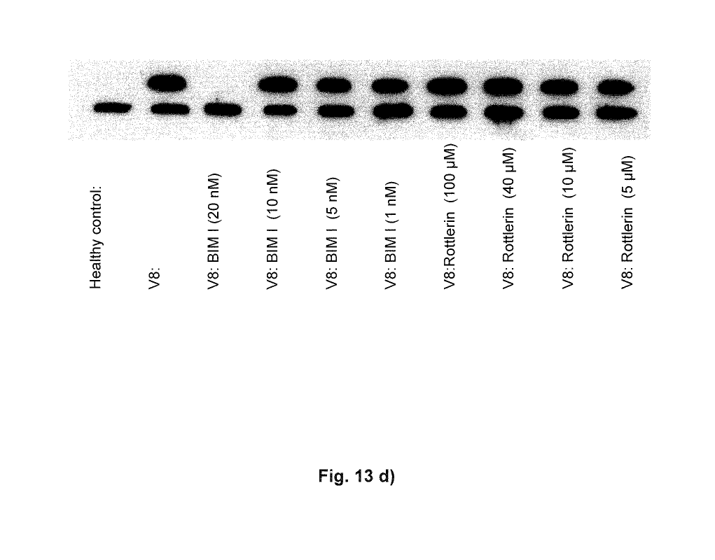

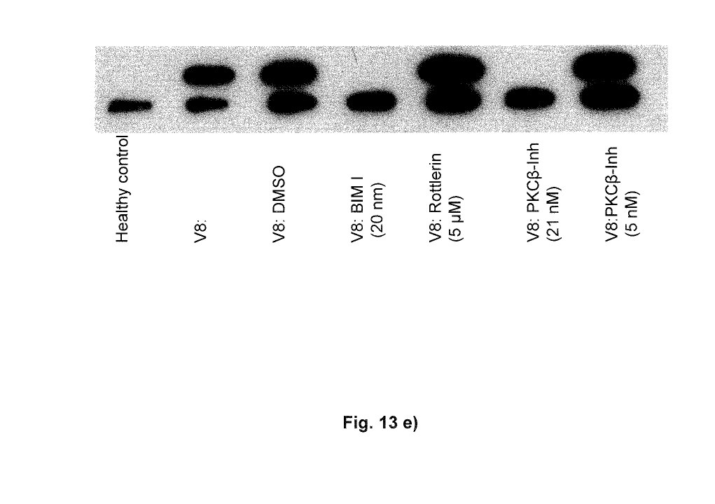

Inhibitory Experiments for the Identification of the Responsible Kinase and Phosphatase (FIGS. 13 a and b and 14 a) and b).

Stable transfected lymphoblastoid cell lines were cultured at 2×10e6 cells/ml in the presence of selected inhibitory compounds as indicated to narrow down the number of candidate kinases responsible for hyperphosphorylation at Ser81. The selected inhibitory compounds for the initial test were: 5 μM Staurosporin (inhibits CK1, PKC and tyrosine kinases), 2.5 μM Staurosporin (inhibits PKC and Tyrosine kinases), 50 nM H89 (inhibits PKA), 200 nM Akt 1/2 Inhibitor (inhibits Akt), 100 nM Purvalanol (inhibits CDK), 100 nM SP600 125 (inhibits JNK), 10 pM Ellagic Acid (inhibits PKC) and 200 nm D4476 (inhibits CK1) [4; 5]. After 3 days, cells were removed and analyzed by isoelectric focusing (IEF) and immunoblot detection. After the identification of PKC as the responsible kinase for the phosphorylation of serine 81, the same procedure was repeated with other selected inhibitory compounds to identify the subclass of PKC. The selected inhibitory compounds for the identification of the PKC subunit were: Staurosporin at 2.5 μM, 15 nM and 30 nM. Bisindolylmaleimide I (BIM I) at 1 nM, 5 nM, 10 nM and 20 nM, Rottlerin at 5 μM, 10 μM, 40 μM and 100 μM and the PKCβ-inhibitor at a concentration of 5 nm specifically for PKCβ-II and at a concentration of 21 nm for PKCβ-I and PKCβ-II.

Therefore PKC was identified as the kinase group responsible for the hyperphosphorylation of progranulin. Moreover we observed in the IEF of LCL lysates of progranulin-antibody positive patients and of healthy controls, whose LCL had been treated with Staurosporin at concentrations of 2.5 pM or 5 pM or Ellagic Acid at a concentration of 10 pM a slight shift of the band of the normal progranulin isoform towards an electronically less negatively charged isoform. Thus a supposed physiologic phosphorylation of the normal progranulin-isoform was inhibited as well by these kinase inhibitors at these high concentrations (FIG. 13 a and b). Furthermore, when administrating Staurosporin at lower concentrations of 15 nM and 30 nM the additional hyper-phosphorylated isoform wasn't detectable as well, but the shift in the electrical charge of the normal progranulin isoform was not observable anymore (FIG. 13 c), indicating that the hyperphosphorylation of Ser81 and the normal phosphorylation of progranulin are caused by different kinases. Moreover, Bisindolylmaleimide I at final concentrations of 1 nM, 5 nM and 10 nM didn't inhibit the hyperphosphorylation, but when further increased to 20 nM the hyper-phosphorylated isoform disappeared. In contrast Rottlerin at concentrations of 5 μM, 10 μM, 40 μM and 100 μM could not inhibit the phosphorylation of Ser81 (FIG. 13 d). When considering the specific inhibition characteristics of Staurosporine, Bisindolylmaleimide I and Rottlerin [4], the results indicated PKCβ-I or -II as the responsible kinase. By the administration of the specific PKCβ-inhibitor at concentrations of 5 nM (inhibits specifically PKCβ-II) or 21 nM (inhibits PKCβ-I and -II) [10] PKCβ-I could be identified as responsible kinase for hyper-phosphorylated progranulin (FIG. 13 e).

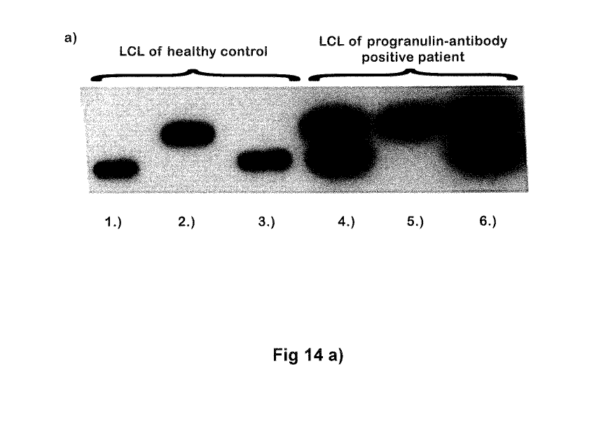

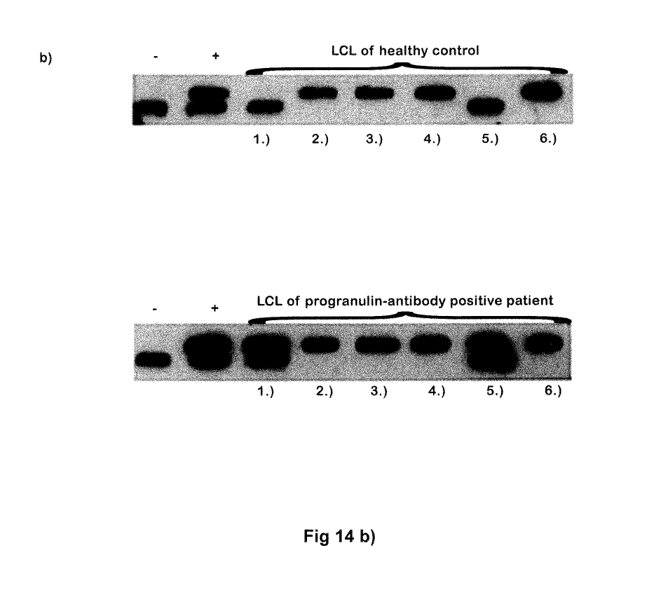

To identify the responsible phosphatase for progranulin's phosphorylated Ser81 a panel of different phosphatase inhibitors was administered at specific concentrations to the cultured LCLs, which had been obtained from progranulin-antibody positive patients and from healthy controls with the aim to inhibit particular groups of phosphatases. This panel consisted of serine/threonine phosphatase inhibitor cocktail, tyrosine phosphatase inhibitor, okadaic acid at 500 nM, 10 nM, 0.1 nM and NIPP1 at 10 pM. Following to this, IEFs of the lysates of these LCLs were performed to check for possible differences in the phosphorylation state. The administration of serine/threonine phosphatase inhibitor cocktail led to the disappearance of the normal progranulin isoform and the exclusive appearance of the hyper-phosphorylated isoform in LCLs of healthy controls and of progranulin-antibody positive patients (FIG. 14 a)). Furthermore the administration of NIPP1 at 10 pM (specific for PP1) and the administration of Ocadaic acid at 0.5 μM and 10 nM (specific for PP1) led to the appearance of the hyperphosphorylated progranulin isoform, but not the administration of less concentrated Ocadaic acid 0.1 nM (specific for PP2A) (FIG. 14 b)).

### Generation of Phosphorylation-Site Specific Fabs

The synthesis of specific fabs with reactivity only against the hyper-phosphorylated progranulin isoform or the normal progranulin isoform was performed as described before [7; 8].

For differential selection N-terminally biotinylated progranulin fragments (70-91 AA) were used either phosphorylated or non-phosphorylated (Ser81). These synthetic peptides had an HPLC-controlled purity higher than 95% (Intavis, Köln, Germany).

Wt-Progranulin (70-91 AA):
(SEQ ID NO: 17)
BIOT-SGGGGS HSCIFTVSGTSSCCPFPEAVA
p-Progranulin (70-91 AA)
(SEQ ID NO: 18)
BIOT-SGGGGS HSCIFTVSGT-S(p)-SCCPFPEAVA

As phagemid library, a non-immune, semi-synthetic human Fab repertoire containing 3.7×10¹⁰ different possible antibody fragments was utilized [9]. Phages were pre-incubated 1 h at room temperature (RT) in 2% non-fat dry milk/PBS. In the second round of selection, phages were pre-incubated with streptavidin-coated paramagnetic beads (Invitrogen and Dynal, Oslo, Norway) to remove streptavidin binders. Additionally to increase the probability to yield phages discriminating between the two progranulin isoforms, phages which should be selected for hyper-phosphorylated progranulin were pre-absorbed with the biotinylated non-phosphorylated progranulin fragment (70-91 AA), and phages which should be selected for normal progranulin, were pre-absorbed with biotinylated p-progranulin fragment (70-91 AA). Phages were subsequently incubated for 45 min in decreasing concentrations of biotinylated phosphorylated or biotinylated non-phosphorylated progranulin fragments (500 nM, 300 nM, 200 nM and 100 nM). Streptavidin beads were then added and mixed with constant rotation (15 min, RT). Then six wash cycles with 2% non-fat dry milk plus 0.1% Tween, followed by these six cycles with 0.1% Tween-PBS and by two additional washes with PBS. Finally bound phages were eluted with 100 mM Triethylamin and neutralized with Tris-HCl pH 7.2. Phages were used to infect E. coli strain TG1 (30 min at 37° C.), and bacteria were grown overnight at 30° C. on agar. To achieve fabs discriminating reliably between hyper-phosphorylated and non-hyper-phosphorylated progranulin six rounds of selection were necessary. The specificity of these fabs for the non-hyper-phosphorylated or the phosphorylated (Ser81) progranulin isoform is verified by IEF.

### Western Immunoblotting of Progranulin

For western immunoblot, whole blood samples were centrifuged and washed with phosphate buffer solution (PBS) and the pellet treated with lysis buffer (8 M urea, 0.1 M NaH2PO4, 0.01 M Tris HCl, 0.1% NP40) at room temperature for 15 min. Equivalent amounts of cell lysates from progranulin-antibody negative, progranulin antibody positive patients and from healthy controls were loaded and separated in 10% SDS-PAGEs or 8% -15% gradient SDS-PAGEs, then transferred to PVDF membranes using a transblot semidry transfer cell (Bio Rad). The membranes were blocked in TST/milk buffer (10% milk in 10 mM Tris/HCl, pH 7.5, 150 mM NaCl, 0.1% [v/v] Tween 20) overnight, washed, and incubated for 1 hour with patient's plasma, which is directed against the N-terminal 112 AA and was diluted 1:1,000 in TST or with HRP-labeled rabbit anti-human progranulin IgG which is directed against the C-terminus of progranulin and was diluted 1:1,000 (BIOSS) or with fabs specific for the phosphorylated (Ser81) or the non phosphorylated (Ser81) N-terminus, i.e. 70-91AA. After 3 washings in TST, the membranes previously incubated with patient's plasma were incubated for 1 hour at room temperature with PDX-labeled goat anti-human IgG Ab (Dianova) diluted 1:5,000 in TST, subsequently washed in TST followed by ECL development. The membranes incubated with HRP-labeled rabbit anti-human progranulin IgG, were directly washed in TST followed by ECL development. And the membranes incubated with fabs specific for phosphorylated or non-phosphorylated Ser81 were incubated with HRP-labeled anti-human Fab Ab (Dianova) diluted 1:5000 in TST, subsequently washed in TST followed by ECL development.

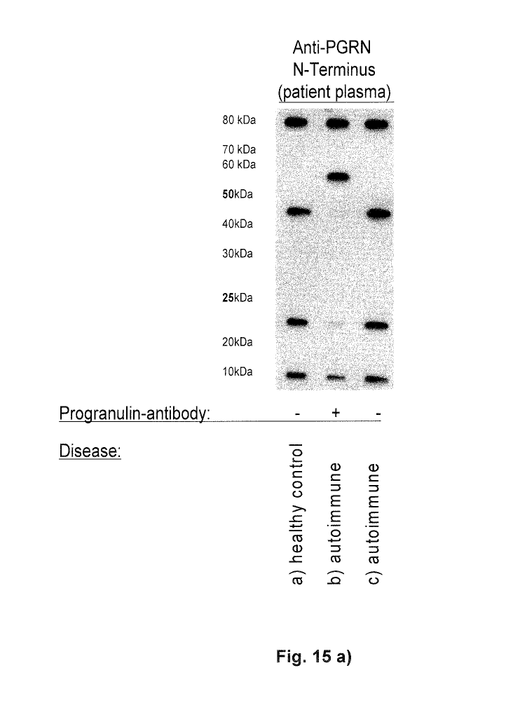

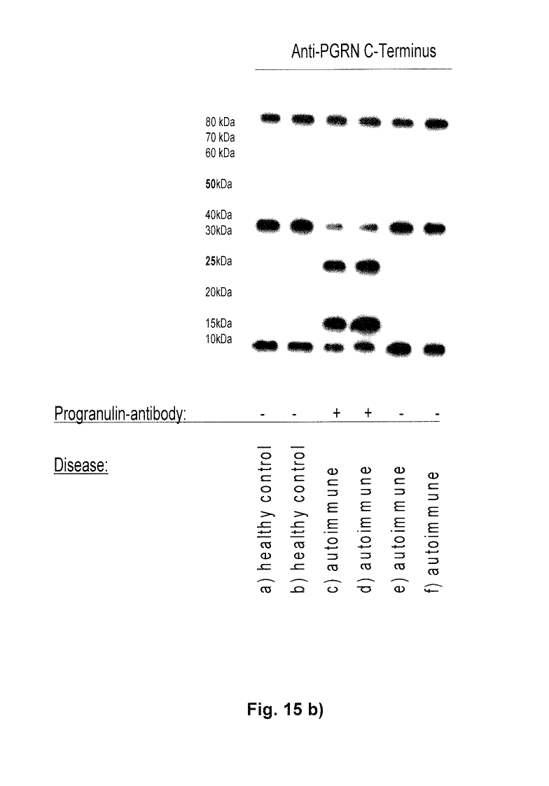

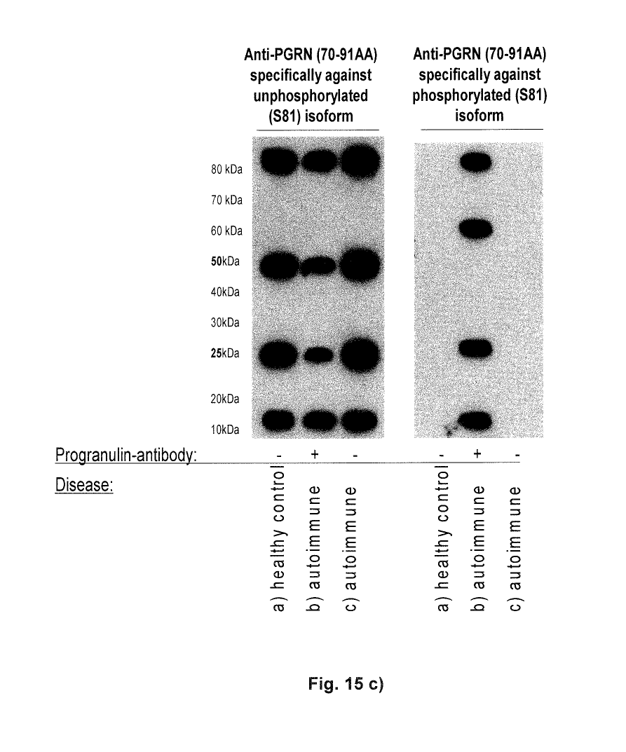

Western-Immunoblotting of Progranulin Aberrant Pathway (FIGS. 15s and 16)

Western-immunoblotting of whole blood cell lysates with a gradient SDS PAGE, revealed a different conversion pattern of progranulin to mature granulins in progranulin-antibody positive patients compared to progranulin-antibody negative patients or healthy controls. In detail, there appeared a longer N-terminal (55 kDa instead of 45 kDa) (FIG. 15 a)) and correspondingly a shorter c-terminal conversion fragment (25 kDa instead of 35 kDa) (FIG. 16b)) in patients with progranulin autoantibodies. Moreover Western-Blots performed with fabs, which had a specific affinity against the hyper-phosphorylated or the non-hyper-phosphorylated N-terminal progranulin, revealed that only the hyperphosphorylated progranulin isoform was converted aberrantly (FIG. 15 c)), indicating a shift in the initial conversion region of hyperphosphorylated progranulin form in-between A and C to in-between C and D (FIG. 15 a) and b)) These data could be confirmed by western-blots of progranulin expressed with N-terminal His- and C-terminal FLAG-tags in LCLs of progranulin-antibody-positive patients, and of progranulin-antibody negative patients and of healthy controls (FIG. 17).

Detection of Hyperphosphorylated Progranulin (pPGRN) Plasma Levels

Nunc maxisorb plates were coated overnight at 4° C. with Rabbit anti human PGRN (C-Terminus) at a dilution of 1:2,500 (v/v; LsBio, Seattle Wash., USA). Blocking was performed with 1.5% (w/v) gelatin in TBS and washing steps were performed with TBS-Tx [TBS, 0.1% (v/v) Tx100]. The individual sera were utilized non-diluted. ELISA was performed according to standard protocols. For the detection of the hyperphosphorylated progranulin, a Ser81 phosph-specific Anti-PGRN fab was utilized at a concentration of 10 μg/l. This fab had been synthesized before by us. Following to this, correspondent biotinylated anti human fab secondary antibodies were used for the immunoassays. Peroxidase-labeled streptavidin (Roche) was used at a dilution of 1:50,000.

### Staining and Flow Cytometry of Hyperphosphorylated PGRN.

Staining of hyperphosphorylated PGRN was performed on blood smears and by flow cytometry analysis of PBMCs derived from PGRN-Ab-positive and negative patients. Blood smears were fixated for 10 min in 4% PFA, followed by 0.5% Tx100 for 10 min at room temperature. For inhibition of endogenous peroxidase to reduce unspecific background staining, blood smears were pretreated with 3% hydrogen peroxide diluted in methanol for 10 min at room temperature. Finally blocking with 5% BSA was performed for 30 min at room temperature. Myc-tagged Fabs specific either for phosphorylated 81 PGRN or non-phosphorylated PGRN were used at a concentration of 10 μg/ml followed by incubation with mouse anti-myc antibody (1 μg/ml), biotinylated anti mouse IgG antibody (1:500) and streptavidin-Pox (1:10.000). Phospho-unspecific staining of PGRN was conducted with a commercially available goat anti human PGRN antibody (1:1.000) followed by biotinylated anti goat IgG antibody (1:1.000) and streptavidin-Pox (1:10.000). Staining for Actin (1:40) was followed by biotinylated anti rabbit IgG antibody (1:1.000) and streptavidin-Pox (1:10.000). For development AEC was applied as substrate, and counterstaining was performed with hematoxylin. For flow cytometry analysis of PBMCs peripheral blood of PGRN-antibody positive and negative patients was subjected to Ficoll separation according to standard protocols. Cells were fixated in 2% PFA for 20 min at room temperature, followed by permeabilization in 0.5% Saponin for 20 min at room temperature. Staining of PBMCs was performed with myc-tagged Fabs either specific for phosphorylated Ser81 PGRN or for wild type PGRN, followed by mouse anti-myc antibody (1 μg/ml), biotinylated anti mouse IgG antibody (1:500) and Streptavidin-APC at a dilution of 1:500. Control stainings were performed with rabbit anti human Actin antibodies (1:100) followed by biotinylated anti rabbit IgG antibody (1:500) or with a phospho-unspecific commercially available PGRN antibody (5 μg/ml), followed by incubation with biotinylated anti goat IgG antibody (1:500) and incubation with Strept-APC (1:500). Western blotting of PGRN and PGRN conversion products. Equal amounts of whole blood cell lysates from PGRN-Ab-negative, PGRN-Ab-positive patients and from healthy controls were loaded and separated in 10% SDS-PAGEs or 8%-15% gradient SDS-PAGEs, then transferred to PVDF membranes using a transblot semidry transfer cell (Bio Rad). The membranes were blocked overnight in TST/milk buffer (10% milk in 10 mM Tris/HCl, pH 7.5, 150 mM NaCl, 0.1% [v/v] Tween 20), 15″ “washed, and incubated for 1 h with patient's plasma, which is directed against the N-terminal 112 AA (1) or with HRP-labeled rabbit anti-human PGRN IgG which is directed against the C-terminus of PGRN and was diluted (BIOSS) (both diluted 1:1,000 in TST) or with the selected phospho-specific and non-phospho specific anti-PGRN Fabs to study specifically the N-terminal conversion fragments of hyperphosphorylated and normal PGRN. After 3 washings in TST, the membranes previously incubated with patient's plasma were incubated for 1 h at room temperature with PDX-labeled goat anti-human IgG Ab (Dianova) diluted 1:5,000 in TST. The membranes incubated with phospho-specific anti-PGRN Fabs were washed in TST followed by incubation with HRP-labeled goat anti-human Fab Ab (Dianova) diluted 1:5,000 and subsequently followed by chemiluminescence development. Furthermore, the conversion pattern of recombinant full-length PGRN or fragments thereof with N-terminal HIS- and C-terminal FLAG-tags expressed in LCLs derived of PGRN-Ab-positive patients and of controls was performed. LCLs were washed 3 times with PBS and subsequently treated with lysis buffer (10 mM Tris HCl pH 8, 30 minutes 4° C.). The samples and controls were then submitted to Western immunoblotting with SDS gradient-gels (8%-15%).

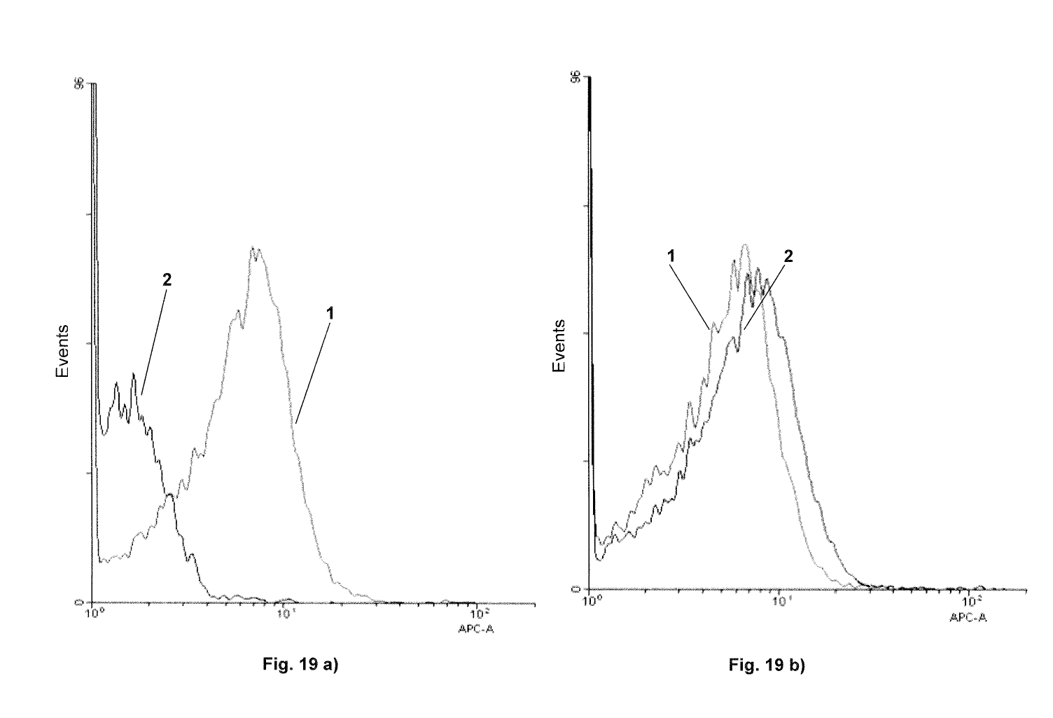

Staining of cytospins and bloodsmears from PGRN-Ab-positive patients for the non-phosphorylated PGRN isoform showed a comparable presence of the non-hyperphosphorylated PGRN isoform as in PGRN-Ab-negative patients. Staining of cytospins and bloodsmears from a PGRN-Ab-positive patients for the hyperphosphorylated progranulin isoform showed strong presence of the hyperphosphorylated PGRN isoform in contrast to the absence in PGRN-Ab negative patients. Phosphorylation specific flow cytometric analysis of progranulin of PBMCs from PGRN-Ab positive or negative patients showed equal presence of actin and the non-hyperphosphorylated PGRN isoform on the cell membranes of PGRN-Ab-positive and negative patients. In contrast the hyperphosphorylated PGRN isoform was only detected on PGRN-Ab positive patients (FIG. 19 a and b).

### REFERENCE LIST

(1) Tang W, Lu Y, Tian Q Y, Zhang Y, Guo F J, Liu G Y, et al. The growth factor progranulin binds to TNF receptors and is therapeutic against inflammatory arthritis in mice. Science 2011 Apr. 22; 332(6028):478-84.
(2) Neumann F, Wagner C, Preuss K D, Kubuschok B, Schormann C, Stevanovic S, et al. Identification of an epitope derived from the cancer testis antigen HOM-TES-14/SCP1 and presented by dendritic cells to circulating CD4+T cells. Blood 2005 Nov. 1; 106(9):3105-13.
(3) Grass S, Preuss K D, Ahlgrimm M, Fadle N, Regitz E, Pfoehler C, et al. Association of a dominantly inherited hyperphosphorylated paraprotein target with sporadic and familial multiple myeloma and monoclonal gammopathy of undetermined significance: a case-control study. Lancet Oncol 2009 October; 10(10):950-6.
(4) Merck KGaA, Darmstadt G. Calbiochem Inhibitor Sourcebook. 2009.
(5) Held G, Wadle A, Dauth N, Stewart-Jones G, Sturm C, Thiel M, et al. MHC-peptide-specific antibodies reveal inefficient presentation of an HLA-A*0201-restricted, Melan-A-derived peptide after active intracellular processing. Eur J Immunol 2007 July; 37(7):2008-17.
(6) Chapman P B, Lester T J, Casper E S, Gabrilove J L, Wong G Y, Kempin S J, et al. Clinical pharmacology of recombinant human tumor necrosis factor in patients with advanced cancer. J Clin Oncol 1987 December; 5(12):1942-51.
(7) Held G, Wadle A, Dauth N, Stewart-Jones G, Sturm C, Thiel M, et al. MHC-peptide-specific antibodies reveal inefficient presentation of an HLA-A*0201-restricted, Melan-A-derived peptide after active intracellular processing. Eur J Immunol 2007 July; 37(7):2008-17.
(8) Preuss K D, Pfreundschuh M, Fadle N, Regitz E, Raudies S, Murwaski N, et al. Hyperphosphorylation of autoantigenic targets of paraproteins is due to inactivation of PP2A. Blood 2011 Sep. 22; 118(12):3340-6.
(9) de Haard H J, van N N, Reurs A, Hufton S E, Roovers R C, Henderikx P, et al. A large non-immunized human Fab fragment phage library that permits rapid isolation and kinetic analysis of high affinity antibodies. J Biol Chem 1999 Jun. 25; 274(26):18218-30.
(10) Tanaka M, Sagawa S, Hoshi J, Shimoma F, Matsuda I, Sakoda K, et al. Synthesis of anilino-monoindolylmaleimides as potent and selective PKCbeta inhibitors. Bioorg Med Chem Lett 2004 Oct. 18; 14(20):5171-4.
(11) Zhu J, Nathan C, Jin W, Sim D, Ashcroft G S, Wahl S M, et al. Conversion of proepithelin to epithelins: roles of SLPI and elastase in host defense and wound repair. Cell 2002 Dec. 13; 111(6):867-78.

1. An in vitro method for detection of anti-progranulin-autoantibodies in a biological sample comprising use of progranulin or an antigenic progranulin peptide/fragment or variant thereof.

2. (canceled)

3. The method of claim 1 comprising steps of:
a) contacting the biological sample with
1) hyperphosphorylated progranulin and/or hyperphosphorylated antigenic peptide/fragment and/or variant thereof; or
2) progranulin and/or antigenic peptide/fragment and/or variant thereof; or
3) a mixture of 1) and 2)
b) detecting the binding of anti-progranulin autoantibodies
1′) to said hyperphosphorylated progranulin and/or hyperphosphorylated antigenic peptide/fragment and/or variant thereof or
2′) to said progranulin and/or to said antigenic peptide/fragment thereof and/or to said variant thereof or
3′) to said mixture of 1) and 2); and
c) optionally detecting in the biological sample one or more autoantibodies or autoantigens which are indicative of an autoimmune disorder.

4. The method of claim 3, wherein the biological sample is from an individual suspected of having or at a risk of an autoimmune disease.

5. The method of claim 3 wherein the method is for the diagnosis of an autoimmune disease and the presence of anti-progranulin-autoantibodies in the biological sample is indicative of an autoimmune disease.

6. The method of claim 3 wherein progranulin comprises or consists of the amino acid sequence set forth in SEQ ID NO: 1 or a antigenic fragment thereof.

7. The method of claim 3 wherein the hyperphosphorylated progranulin comprises or consists of the amino acid sequence set forth in SEQ ID NO: 1 which is hyper-phosphorylated or a hyper-phosphorylated fragment thereof.

8. The method of claim 3 wherein the antigenic peptide/fragment or variant thereof comprises or consists of the N-terminal 120 amino acid region of progranulin or fragment thereof; N-terminal 120 amino acid region of SEQ ID NO 1 or fragment thereof; the N-terminal 112 amino acid region of SEQ ID NO 1, or SEQ ID NO 2.

9. The method of claim 3 wherein the hyperphosphorylated antigenic peptide/fragment comprises or consists of the N-terminal 120 amino acid region of progranulin or fragment thereof; N-terminal 120 amino acid region of SEQ ID NO 1 or fragment thereof; the N-terminal 112 amino acid region of SEQ ID NO 1 or fragment thereof, or SEQ ID NO 2 or fragment thereof, wherein said region or fragment thereof or said SEQ ID NO 2 are hyper-phosphorylated or hyper-phosphorylated fragment or hyper-phosphorylated variant thereof.

10. The method of claim 3, wherein the hyperphosphorylated progranulin or the hyperphosphorylated antigenic peptide/fragment or hyperphosphorylated variant thereof comprises a hyper-phosphorylated serine.

11-38. (canceled)

39. A method for treating an immune disorder in a subject, comprising administering an immune modulator to a subject, said subject having anti-progranulin autoantibodies based on diagnosis, wherein administering ameliorates, prevents, or treats the autoimmune disorder in the subject.

40. The method of claim 39, wherein the immune modulator is an anti-TNF blocker optionally selected from Infliximab, Certolizumab pegol, Adalimumab, Etanercept or Atsttrin.

41. (canceled)

42. The method of claim 39, wherein the autoimmune disease is selected from the group consisting of vasculitis, arthritides, autoimmune diseases of the connective tissue, inflammatory bowel diseases, autoimmune diseases of the liver and the bile duct, autoimmune disease of the thyroid gland, dermatologic autoimmune diseases, neurologic immune diseases, and Diabetes type I.

43. The method according to claim 42 wherein
vasculites are selected from medium to small vessel vasculitis and large vessel vasculitis, arthritides are selected from seronegative and seropositive rheumatoid arthritis, psoriatic arthritis, Bechterew's disease, and juvenile idiopathic arthritis,
inflammatory bowel diseases are selected from Crohn's disease and ulcerative colitis, diseases of the liver and the bile duct are selected from autoimmuno-hepatitis, primary biliary cirrhosis, and primary sclerosing Cholangitis,
autoimmune diseases of the thyroid gland are selected from Hashimoto's thyreoiditis, and Grave's disease,
autoimmune diseases of the connective tissue are selected from systemic lupus erythematosus disease, Sjörgen's syndrome, scleroderma, dermato- and poly-myositis, Sharp syndrome, systemic sclerosis and CREST syndrome,
neurologic autoimmune diseases are selected from multiple sclerosis, chronic inflammatory demyelating polyneuropathy (CIDP), and myasthenia gravis.

44. The method according to claim 43, wherein the medium to small vasculitis is selected from classical panarteritis nodosa, granulomatosis with polyangiitis, microscopic panarteritis, Churg-Strauss syndrome, and Behcet's disease and the large vessel vasculitis is selected from giant cell arteritis, polymyalgia rheumatica, and Takayasu's arteritis.

45-55. (canceled)

56. An in vitro method for detection of hyper-phosphorylated progranulin or an antigenic hyper-phosphorylated progranulin peptide/fragment thereof in a biological sample comprising steps of:
a) contacting a biological sample with an antibody which specifically binds to hyper-phosphorylated progranulin, or to a hyper-phosphorylated fragment thereof,
b) detecting the binding of said antibody to said hyper-phosphorylated progranulin or hyper-phosphorylated fragment thereof, and
c) optionally detecting in the biological sample one or more autoantibodies or autoantigens which are indicative of an autoimmune disorder.

57. The method according to claim 56, wherein the method is for the diagnosis of an autoimmune disease and the presence of hyper-phosphorylated progranulin and/or hyper-phosphorylated fragment(s) thereof is indicative of an autoimmune disease.

58. The method of claim 8 wherein the antigenic peptide/fragment or variant thereof comprises or consists of the N-terminal 112 amino acid region of progranulin or fragment thereof.

59. The method of claim 9 wherein the hyperphosphorylated antigenic peptide/fragment comprises or consists of the N-terminal 112 amino acid region of progranulin or fragment thereof.

60. The method of claim 10, wherein the hyperphosphorylated progranulin or the hyperphosphorylated antigenic peptide/fragment or hyperphosphorylated variant thereof comprises serine in position 81 in SEQ ID NO: 1 or the serine corresponding to S81 of SEQ ID NO: 1.


SEQUENCE LISTING (20 sequences)

SEQ ID NO: 1 - protein, 593 aa, Homo sapiens
  misc (1)..(593): Progranulin
MWTLVSWVALTAGLVAGTRCPDGQFCPVACCLDPGGASYSCCRPLLDKWPTTLSRHLGGPCQVDAHCSAGHSCIFTVSGTSSCCPFPEAVACGDGHHCCPRGFHCSADGRSCFQRSGNNSVGAIQCPDSQFECPDFSTCCVMVDGSWGCCPMPQASCCEDRVHCCPHGAFCDLVHTRCITPTGTHPLAKKLPAQRTNRAVALSSSVMCPDARSRCPDGSTCCELPSGKYGCCPMPNATCCSDHLHCCPQDTVCDLIQSKCLSKENATTDLLTKLPAHTVGDVKCDMEVSCPDGYTCCRLQSGAWGCCPFTQAVCCEDHIHCCPAGFTCDTQKGTCEQGPHQVPWMEKAPAHLSLPDPQALKRDVPCDNVSSCPSSDTCCQLTSGEWGCCPIPEAVCCSDHQHCCPQGYTCVAEGQCQRGSEIVAGLEKMPARRASLSHPRDIGCDQHTSCPVGQTCCPSLGGSWACCQLPHAVCCEDRQHCCPAGYTCNVKARSCEKEVVSAQPATFLARSPHVGVKDVECGEGHFCHDNQTCCRDNRQGWACCPYRQGVCCADRRHCCPAGFRCAARGTKCLRREAPRWDAPLRDPALRQLL

SEQ ID NO: 2 - protein, 112 aa, Homo sapiens
  misc (1)..(112): N-terminal 1-112 amino acids of progranulin
MWTLVSWVALTAGLVAGTRCPDGQFCPVACCLDPGGASYSCCRPLLDKWPTTLSRHLGGPCQVDAHCSAGHSCIFTVSGTSSCCPFPEAVACGDGHHCCPRGFHCSADGRSC

SEQ ID NO: 3 - DNA, 1782 bp, Homo sapiens
  misc (1)..(1782): Progranulin
atgtggaccctggtgagctgggtggccttaacagcagggctggtggctggaacgcggtgcccagatggtcagttctgccctgtggcctgctgcctggaccccggaggagccagctacagctgctgccgtccccttctggacaaatggcccacaacactgagcaggcatctgggtggcccctgccaggttgatgcccactgctctgccggccactcctgcatctttaccgtctcagggacttccagttgctgccccttcccagaggccgtggcatgcggggatggccatcactgctgcccacggggcttccactgcagtgcagacgggcgatcctgcttccaaagatcaggtaacaactccgtgggtgccatccagtgccctgatagtcagttcgaatgcccggacttctccacgtgctgtgttatggtcgatggctcctgggggtgctgccccatgccccaggcttcctgctgtgaagacagggtgcactgctgtccgcacggtgccttctgcgacctggttcacacccgctgcatcacacccacgggcacccaccccctggcaaagaagctccctgcccagaggactaacagggcagtggccttgtccagctcggtcatgtgtccggacgcacggtcccggtgccctgatggttctacctgctgtgagctgcccagtgggaagtatggctgctgcccaatgcccaacgccacctgctgctccgatcacctgcactgctgcccccaagacactgtgtgtgacctgatccagagtaagtgcctctccaaggagaacgctaccacggacctcctcactaagctgcctgcgcacacagtgggggatgtgaaatgtgacatggaggtgagctgcccagatggctatacctgctgccgtctacagtcgggggcctggggctgctgcccttttacccaggctgtgtgctgtgaggaccacatacactgctgtcccgcggggtttacgtgtgacacgcagaagggtacctgtgaacaggggccccaccaggtgccctggatggagaaggccccagctcacctcagcctgccagacccacaagccttgaagagagatgtcccctgtgataatgtcagcagctgtccctcctccgatacctgctgccaactcacgtctggggagtggggctgctgtccaatcccagaggctgtctgctgctcggaccaccagcactgctgcccccagggctacacgtgtgtagctgaggggcagtgtcagcgaggaagcgagatcgtggctggactggagaagatgcctgcccgccgggcttccttatcccaccccagagacatcggctgtgaccagcacaccagctgcccggtggggcagacctgctgcccgagcctgggtgggagctgggcctgctgccagttgccccatgctgtgtgctgcgaggatcgccagcactgctgcccggctggctacacctgcaacgtgaaggctcgatcctgcgagaaggaagtggtctctgcccagcctgccaccttcctggcccgtagccctcacgtgggtgtgaaggacgtggagtgtggggaaggacacttctgccatgataaccagacctgctgccgagacaaccgacagggctgggcctgctgtccctaccgccagggcgtctgttgtgctgatcggcgccactgctgtcctgctggcttccgctgcgcagccaggggtaccaagtgtttgcgcagggaggccccgcgctgggacgcccctttgagggacccagccttgagacagctgctgtga

SEQ ID NO: 4 - DNA, 27 bp, Artificial
  oligonucleotide derived from human progranulin gene
gatatcatgtggaccctggtgagctgg

SEQ ID NO: 5 - DNA, 27 bp, Artificial
  oligonucleotide derived from human progranulin gene
gatatccagcagctgtctcaaggctgg

SEQ ID NO: 6 - DNA, 30 bp, artificial
  oligonucleotide derived from human progranulin gene
gatatcatggatagtcagttcgaatgcccg

SEQ ID NO: 7 - DNA, 27 bp, artificial
  oligonucleotide derived from human progranulin gene
gatatcatgcaggttgatgcccactgc

SEQ ID NO: 8 - DNA, 48 bp, artificial
  oligonucleotide derived from human progranulin gene and for amplifying His-tag
gatatcatgcatcaccatcaccatcacatgtggaccctggtgagctgg

SEQ ID NO: 9 - DNA, 45 bp, artificial
  oligonucleotide derived from human progranulin gene and for amplifying His-tag
gatatcatgcatcaccatcaccatcacctgagcaggcatctgggt

SEQ ID NO: 10 - DNA, 45 bp, artificial
  oligonucleotide derived from human progranulin gene and for amplifying His-tag
gatatcatgcatcaccatcaccatcacaacaactccgtgggtgcc

SEQ ID NO: 11 - DNA, 45 bp, artificial
  oligonucleotide derived from human progranulin gene and for amplifying His-tag
gatatcatgcatcaccatcaccatcacgtggccttgtccagctcg

SEQ ID NO: 12 - DNA, 24 bp, artificial
  oligonucleotide derived from human progranulin gene and for amplifying His-tag
gatatcgcacttactctggatcag

SEQ ID NO: 13 - DNA, 24 bp, artificial
  oligonucleotide derived from human progranulin gene and for amplifying His-tag
gatatcacaggtacccttctgcgt

SEQ ID NO: 14 - DNA, 24 bp, artificial
  oligonucleotide derived from human progranulin gene and for amplifying His-tag
gatatcctgacactgcccctcagc

SEQ ID NO: 15 - DNA, 30 bp, artificial
  oligonucleotide derived from human progranulin gene
ccccggaggagccgcctacagctgctgccg

SEQ ID NO: 16 - DNA, 30 bp, artificial
  oligonucleotide derived from human progranulin gene
cggcagcagctgtaggcggctcctccgggg

SEQ ID NO: 17 - DNA, 29 bp, artificial
  oligonucleotide derived from human progranulin gene
accgtctcagggactgccagttgctgccc

SEQ ID NO: 18 - DNA, 29 bp, artificial
  oligonucleotide derived from human progranulin gene
gggcagcaactggcagtccctgagacggt

SEQ ID NO: 19 - protein, 27 aa, Homo sapiens
  misc (1)..(27): Progranulin fragment
SGGGGSHSCIFTVSGTSSCCPFPEAVA

SEQ ID NO: 20 - protein, 27 aa, Homo sapiens
  misc (1)..(27): p-Progranulin fragment
SGGGGSHSCIFTVSGTSSCCPFPEAVA